(19) United States
(12) Patent Application Publication
(10) Pub. No.: US 2024/0390303 A1
(43) Pub. Date: Nov. 28, 2024

(54) CHEMICALLY MODIFIED CURCUMINS FOR USE IN THE PRODUCTION OF LIPOXINS

(71) Applicants: The Research Foundation for the State University of New York, Albany, NY (US); Chem-Master International Inc., East Setauket, NY (US)

(72) Inventors: Guirong Wang, Syracuse, NY (US); Xu Yongan, Syracuse, NY (US); Ying Gu, Centereach, NY (US); Hsi-ming Lee, Setauket, NY (US); Lorne M. Golub, Smithtown, NY (US); Francis Johnson, Setauket, NY (US); Yongan Xu, Syracuse, NY (US)

(21) Appl. No.: 18/612,665

(22) Filed: Mar. 21, 2024

Related U.S. Application Data
(63) Continuation of application No. 17/353,504, filed on Jun. 21, 2021, now abandoned.
Application No. 17/353,504 is a continuation of application No. 15/556,441, filed on Sep. 7, 2017, now abandoned.
(60) Provisional application No. 62/171,951, filed on Jun. 5, 2015.
(60) Provisional application No. 62/131,125, filed on Mar. 10, 2015.

(51) Int. Cl. A61K 31/165; A61K 31/12; A61K 31/44; A61K 31/444

(52) U.S. Cl. CPC A61K 31/165; A61K 31/12; A61K 31/44; A61K 31/444

(57) ABSTRACT

A method of increasing production of one or more lipoxins in a subject in need thereof comprising administering to the subject an amount of a compound having the structure:

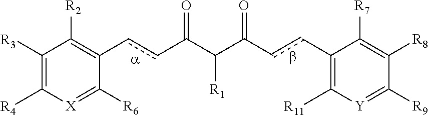

or a pharmaceutically acceptable salt or ester thereof, so as to thereby increase production of the one or more lipoxins in the subject.

This application claims priority of U.S. Provisional Application Nos. 62/171,951, filed Jun. 5, 2015 and 62/131,125, filed Mar. 10, 2015, the contents of each of which are hereby incorporated by reference.

The invention was made with government support under Grant number HL096007 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Curcumin is a naturally occurring compound of the curcuminoid family, isolated originally from the plant Curcuma longa. The rhizome of this plant, specifically, is used to create the spice known as turmeric, and is a major component of the daily diet in many Asian countries. Even before the modern characterization of curcumin's molecular structure and functionality, it has long been used in traditional eastern medicines.

With its natural medicinal history in mind, curcumin has been studied extensively over the past few decades in a wide variety of systems, and has been found to exhibit significant pleiotropic effects. These effects may be attributed to the chemistry of curcumin, consisting of two polyphenolic rings joined by a conjugated, flexible linker region with a R-diketone moiety at its center (FIG. 1). The β-diketone moiety is capable of undergoing keto-enol tautomerization, though the enol form is more stable in both the solid phase and in solution (Gupta, S. C. et al. 2011) and is the dominant species at physiological pH (Gupta, S. C. et al. 2011; Zhang, Y. et al. 2012). The biological activities of curcumin are wide ranging: beyond having intrinsic antioxidant properties, it has been found to bind a wide spectrum of cellular constituents in vitro and in vivo, including inflammatory molecules, protein kinases, carrier proteins, cell survival proteins, structural proteins, the prion protein, antioxidant response elements, metal ions, and more (Gupta, S. C. et al. 2011). In addition, curcumin shows virtually no toxicity in humans (Gupta, S. C. et al. 2011; Ammon, H. P. T. et al. 1991).

While curcumin has been shown to have multiple beneficial effects, its poor oral absorption and lack of solubility in physiological fluid has all but precluded its use as a medicinal substance. Therefore, novel chemically-modified curcumins with enhanced pharmacokinetic and pharmacodynamic properties are needed.

In 1984, Serhan and colleagues discovered the lipoxins, LXA4 and LXB4, by incubating 15 L-hydroperoxy-5,8,11,13-eicosatetraenoic acid (15-HPETE) with human leukocytes. LXA4 and LXB4 biosynthesis was proposed to arise from arachidonic acid via interaction of the 5-lipoxygenase (5-LO) and 15-lipoxygenase (15-LO) pathways (Serhan, C. N. et al. 1984). The biological actions of LXA4 and LXB4 have been characterized in many cell and tissue types, both in vitro and in vivo. The lipoxins provide counterregulatory signals, with particularly potent effects on inflammatory processes that would ultimately combine to promote the resolution of inflammation (Parkinson, J. F. 2006). These effects are achieved by counteracting the effects of pro-inflammatory mediators, such as LTB4, fMLP, platelet activating factor (PAF), LTC4, LTD4, PGE2, TNFα, IL-1β and Il-6 and pathogens on leukocytes, endothelium, epithelium and other cell types. In addition lipoxins can promote the migration of monocytes/macrophages and can stimulate macrophage functions known to be associated with the resolution of Inflammation Parkinson, J. F. 2006).

Reduced levels of LXA4 have been observed in various inflammatory disease including irritable bowel disease, asthma, cystic fibrosis and chronic obstructive pulmonary disease.

Chronic obstructive pulmonary disease (COPD) is a progressive lung disorder characterized by inflammation/fibrosis of the small airways, airway obstruction with increased mucus secretion, emphysema, and abnormal inflammatory response to external stimuli. COPD is the third-leading cause of death in the United States. PM_{2.5}, one of the most dangerous components of air pollution, causes a great health risk. Due to its small size (<2.5 μm), it can reach alveolar spaces of the lung and induce lung inflammation. CMC 2.24, a novel compound from chemically modified curcumin, has been found to be of higher bioactivity, better solubility and no evidence of toxicity compared to Curcumin (Sajjan, U., et al. 2009; Ganesan, S., et al. 2012; Ganesan, S., et al 2010; Le Quement, C., et al. 2008)

The lung matrix is a complex network of proteins and glycoproteins that includes multiple types of collagens, elastin, fibronectin, laminin, and several heparin and sulfate proteoglycans (Elkington, P. T. et al. 2006). Available data indicate that the prevalence of physiologically defined COPD in adults aged 240 years is 9-10% (Halbert, R. J. et al. 2006; Churg A. M. et al. 2008). COPD is the fourth leading cause of death worldwide and the third leading cause of death in the United States. It has been projected to be the third-leading cause of total mortality worldwide and the 5th leading cause of disability by 2020 (Murray, C. J. and Lopez, A. D. 1997; Burney, P. et al. 2014; Vestbo, J. et al. 2013).

Bacterial pneumonia is one of the major causes of acute lung injury (ALI) and acute respiratory distress syndrome (ARDS) (Clement, C. G. et al. 2008). ALI and ARDS are life-threatening condition with an incidence of 79 per 100,000 in the United States (Otto, M. 2010). Staphylococcus aureus is a common gram-positive and opportunistic pathogen, which causes half a million infections a year including pneumonia and approximately 20,000 deaths per year in the United States (Ottto, M. 2010; Klevens, R. M. et al. 2007; Bai, A. D. et al. 2015a; Bai, A. D. et al. 2015b).

Surfactant deactivation has been shown to be an important mechanism for propagation of lung injury. Alveolar Type TI epithelial cells in the lung secrete four surfactant proteins that are distributed on the surface of the alveoli. The hydrophobic surfactant protein B (SP-B) is of particular importance (Ma, C. C. et al. 2012; Pires-Neto, R. C. et al. 2013). SP-B gene expresses two protein products, SP-B^{M} and SP-B^{M}, involved in lowering surface tension and host defense respectively (Yang, L. et al. 2010). The main function of SP-B^{M} protein is to form the monolayer of phospholipids on the surface of alveoli to reduce the surface tension, preventing the collapse of alveoli and maintaining respiration. SP-B^{M} functions as host defense molecule which plays a role in pulmonary bacterial clearance (Yang, L. et al. 2010). Human SP-B gene has an important single nucleotide polymorphism (SNP rs1130866 i.e. SP-B C/T1580) in the N-terminal sapolin-like domain which produces SP-B^{M} protein. The SP-B C/T1580 polymorphism forms two common genetic alleles, SP-B C and T alleles, with differing ability to maintain respiratory homeostasis and host defense (Ma, C. C. et al. 2012). Wang et al. has shown in an in vitro study that proteins from SP-B C and T alleles contain different posttranslational modifications, e.g. SP-B C allele has one additional glycosylation site compared to the T allele. This altered glycosylation may impact protein processing and function (Wang, G. et al. 2003).

### SUMMARY OF THE INVENTION

The present invention provides a method of treating a subject afflicted with a disease or condition comprising administering to the subject an amount of a compound having the structure:

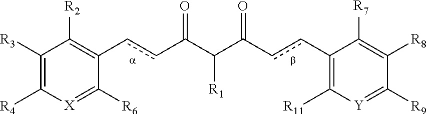

wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃ —SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂ —P(═O)(OR₁₂)(OR₁₃), or —P(OR₁₂)(OR₁₃),
   wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
   R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

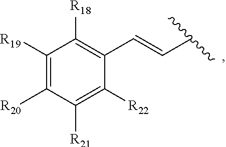

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₁, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl:
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R, is H, then R₂, R₄, R₅, R₈, R₉, or R₁₀, is halogen, —NO₂, —CN, —NR₂₈R₂₉, —NHR₂₈R₂₉, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt or ester thereof, so as to thereby treat the subject, wherein the disease or condition is selected from chronic inflammation, chronic inflammatory disease, rheumatoid arthritis, psoriatic arthritis, osteoarthritis, periodontitis, inflammatory bowel disease, irritable bowel syndrome, psoriasis, ankylosing spondylitis, Sjogren's syndrome, multiple sclerosis, ulcerative colitis, Crohn's disease, systemic lupus erythematosus, lupus nephritis, psoriasis, celiac disease, vasculitis, atherosclerosis, cystic fibrosis, asthma, chronic obstructive pulmonary disease (COPD), bacterial pneumonia, pulmonary bacterial pneumonia, chronic bronchitis, emphysema, chronic and acute lung inflammatory disease, pneumonia, asthma, acute lung injury, lung cancer, diabetes and pulmonary impairment.

The present invention provides a method of increasing production of one or more lipoxins in a subject in need thereof comprising administering to the subject an amount of a compound having the structure:

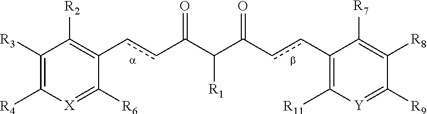

wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH) NR₁₂R₁₃ —SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂ —P(═O)(OR₁₂)(OR₁₃), or —P(OR₁₂)(OR₁₃),
   wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
   R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

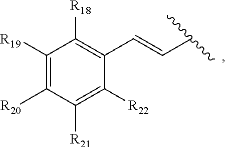

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO—, —CN, —NR₂₃R₂₄, —SR₂₃, —SO₂R₂₃, —COR₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt or ester thereof, so as to thereby increase production of the one or more lipoxins in the subject.

The present invention also provides a method of treating a subject afflicted with a disease associated with decreased levels of one or more lipoxins comprising inducing production of the one or more lipoxins in the subject by administering to the subject an amount of a compound having the structure:

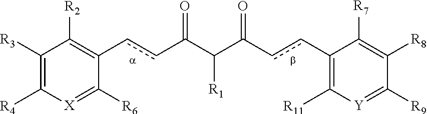

wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH) NR₁₂R₁₃ —SR₁₂, —SO₂R₁₃, —COR₁₄, —CSR₁₄, —C(═NR₁₂)R₁₄, —C(═NR₁₂) NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
   wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
   R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

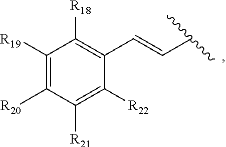

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R, is H, then R₃, R₄, R₅, R₈, R₉, or R₁₀, is halogen, —NO₂, —CN, —NR₂₉R₂₉, —NHR₂₈R₂₉⁺, —SR₂a, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt or ester thereof, so as to thereby treat the subject afflicted with the disease.

### BRIEF DESCRIPTION OF THE FIGURES

FIG. 1A: Effect of in vivo CMC 2.24 treatment on abnormal peritoneal macrophage and/or PMN accumulation in diabetic rats. Thioglycollate- or glycogen-elicited PEs were collected at 4 days or 4 hours prior to sacrifice, respectively, to harvest these acute and chronic inflammatory cells.

FIG. 1B: Effect of in vivo CMC 2.24 treatment on abnormal peritoneal macrophage and/or PMN accumulation in diabetic rats. Thioglycollate- or glycogen-elicited PEs were collected at 4 days or 4 hours prior to sacrifice, respectively, to harvest these acute and chronic inflammatory cells.

FIG. 2A: Effect of in vivo CMC 2.24 on peritoneal macrophage and/or PMNs in cell culture. Cells were cultured in serum-free media (37° C., 5% CO₂/95% O₂ 18 hours), and cell migration were analyzed by Boyden Chamber Assays using CM from LPS-stimulated macrophage as chemoattractant for macrophage and NfMLP for PMN migration.

FIG. 2B: Effect of in vivo CMC 2.24 on peritoneal macrophage and/or PMNs in cell culture. Cells were cultured in serum-free media (37° C., 5% CO₂/95% O₂ 18 hours), and cell migration were analyzed by Boyden Chamber Assays using CM from LPS-stimulated macrophage as chemoattractant for macrophage and NfMLP for PMN migration.

FIG. 3A: Effect of orally administered CMC 2.24 on levels of IL-6 cytokines secreted by peritoneal macrophages from diabetic rats. Thioglycollate-induced peritoneal macrophages were isolated as described herein. Cells were cultured in serum-free media (37° C., 5% CO₂/95% O₂ 18 hours), conditioned media were analyzed for cytokine levels by ELISA.

FIG. 3B: Effect of orally administered CMC 2.24 on levels of IL-β cytokines secreted by peritoneal macrophages from diabetic rats. Thioglycollate-induced peritoneal macrophages were isolated as described herein. Cells were cultured in serum-free media (37° C., 5% CO₂/95% O₂ 18 hours), conditioned media were analyzed for cytokine levels by ELISA.

FIG. 4: Effect of in vivo CMC 2.24 on levels of MMP-2 and MMP-9 in rat peritoneal exudates and PE macrophages. STZ-diabetic rats were administered daily by oral gavage CMC2.24 (30 mg/kg) for 3 weeks. 4 days prior to sacrifice, rats were injected intraperitoneally with 3% thioglycollate, and the peritoneal exudates were collected on the day of sacrifice. Gelatinase activities in the peritoneal exudates or in macrophages were analyzed by gelatin zymography.

FIG. 5: Effect of in vivo CMC 2.24 treatment on abnormal peritoneal macrophage accumulation in diabetic rats. Resident PE (Day 0) were collected prior to sacrifice. Thioglycollate elicited PEs were collected at 4 or 6 days prior to sacrifice, respectively, to harvest macrophages. The cells were counted as described in Methods section.

FIG. 6A: Effect of in vivo CMC 2.24 treatment on levels of MMP-2 and MMP-9 in rat peritoneal CFE at Day 0. STZ-diabetic rats were administered daily by oral gavage CMC2.24 (30 mg/kg) for 3 weeks. Resident peritoneal CFE (Day 0) were collected prior to sacrifice. Gelatinase activities were analyzed by gelatin zymography and scanned by densitometer.

FIG. 6B: Effect of in vivo CMC 2.24 treatment on levels of MMP-2 and MMP-9 in rat peritoneal CFE at Day 4. STZ-diabetic rats were administered daily by oral gavage CMC2.24 (30 mg/kg) for 3 weeks. 4 days prior to sacrifice, rats were injected intraperitoneally with 3% thioglycollate, and the peritoneal exudates were collected on the day of sacrifice. Gelatinase activities were analyzed by gelatin zymography and scanned by densitometer.

FIG. 6C: Effect of in vivo CMC 2.24 treatment on levels of MMP-2 and MMP-9 in rat peritoneal CFE at Day 6. STZ-diabetic rats were administered daily by oral gavage CMC2.24 (30 mg/kg) for 3 weeks. 6 days prior to sacrifice, rats were injected intraperitoneally with 3% thioglycollate, and the peritoneal exudates were collected on the day of sacrifice. Gelatinase activities were analyzed by gelatin zymography and scanned by densitometer.

FIG. 7A: Effect of in vivo CMC 2.24 treatment on levels of IL-10 in rat PE-Day 0. STZ-diabetic rats were administered daily by oral gavage CMC2.24 (30 mg/kg) for 3 weeks. Macrophages from PE were collected and cultured for 18 hours. Serum-free conditioned medium (SFCM) were collected. Resident peritoneal CFE were collected as well. IL-10 levels in SFCM and CFE were analyzed by ELISA.

FIG. 7B: Effect of in vivo CMC 2.24 treatment on levels of IL-10 in rat PE-Day 0. STZ-diabetic rats were administered daily by oral gavage CMC2.24 (30 mg/kg) for 3 weeks. Macrophages from PE were collected and cultured for 18 hours. Serum-free conditioned medium (SFCM) were collected. Resident peritoneal CFE were collected as well. IL-10 levels in SFCM and CFE were analyzed by ELISA.

FIG. 8: Effect of in vivo CMC 2.24 treatment on levels of IL-10 in rat serum—Day 0. STZ-diabetic rats were administered daily by oral gavage CMC2.24 (30 mg/kg) for 3 weeks. Blood were collected prior sacrifice. IL-10 levels in rat serum were analyzed by ELISA.

FIG. 9: Effect of in vivo CMC 2.24 treatment on levels of IL-10 in rat peritoneal CFE-Day 6. STZ-diabetic rats were administered daily by oral gavage CMC2.24 (30 mg/kg) for 3 weeks. Thioglycollate elicited PEs were collected at 6 days after thioglycollate injection, on the day of sacrifice. IL-10 levels in CFE were analyzed by ELISA.

FIG. 10: The effect of high glucose (550 mg/dL) & P. gingivalis LPS (endotoxin) on IL-10 secretion by macrophages from normal (NDC) rats. CMC2.24 was added to the cultures at 0, 2, and 5 μM final concentrations. Each value represents the mean of 3 cultures ±S.E.M.

FIG. 11: Lipoxin A4 secretion by rat “resident” (time 0; before thioglycollate injection) peritoneal macrophages. Peritoneal macrophages were collected from normal & diabetic rats (±CMC2.24 in vivo treatment; n=6 rats/group) in 10 ml PBS/EDTA wash. Adherent cells (m∘s were cultured for 18 hrs, 37° C., the supernatant were analyzed for cytokines. Each value represents the mean±S.E.M.

FIG. 12A: Lipoxin A4 levels in 9a) serum and (b) “Resident” peritoneal wash-fluid (before thioglycollate injection). These fluids were collected from normal & diabetic rats (t CMC2.24 treatment in vivo; n=6 rats/group) and analyzed for lipoxins.

FIG. 12B: Lipoxin A4 levels in 9a) serum and (b) “Resident” peritoneal wash-fluid (before thioglycollate injection). These fluids were collected from normal & diabetic rats (t CMC2.24 treatment in vivo; n=6 rats/group) and analyzed for lipoxins

FIG. 13: Molecular structures of curcumin, CMC2.2, CMC2.24, CMC2.4, and CMC2.5

FIG. 14: Histological changes in the lungs of elastase/LPS-treated mice. Elastase/LPS-treatment induced airway and lung parenchymal inflammation. Formalin-fixed, paraffin-embedded lung tissues harvested from elastase/LPS-treated mice, were stained with hematoxylin and eosin (H&E). Panels A and B show widening of the airspaces consistent with emphysema. Panels C and D show aggregations of neutrophils and mononuclear inflammatory cells in the perivascular and peribronchiolar spaces (Black Arrow). Panels E and F show increased numbers of PAS-positive cells in both the large and small airways.

FIG. 15: Histopathological analysis of lung injury in the four different groups of mice. Histological sections from different groups were stained with H/E and quantified according to lung injury scoring system. The histopathological lung injury score system showed that elastase/LPS-treated mice (Panel B) have a significantly higher lung injury score (P<0.01; E) than the control mice (Panel A). COPD mice challenged with PM_{2.5} (Panel C) have a significantly higher score (P<0.01; E) than the control mice (Panel A). Treating PM_{2.5}-challenged mice with CMC 2.24 (Panel D) show a significant reduction in the lung injury score (P<0.01; E). Graphs represent the mean±SEM. *p<0.05, ** p<0.01 (n=4-6 mice/group).

FIG. 16: Assessment of emphysematous changes in COPD mice by measuring chord length. Lungs of saline-, or elastase/LPS-treated mice were inflated with an equal volume of formalin, processed for paraffin embedding, and stained with H&E. The development of pulmonary emphysema was assessed by measuring the chord length (mean linear intercepts: Lm). The latter are significantly increased (P<0.05) in the lungs of elastase/LPS-treated mice (Panel B). This change was reduced significantly (P<0.05) in the elastase/LPS-treated mice that were administered CMC 2.24 by oral gavage for seven days (Panel C), which returned chord length measurement back to “control” levels. Graphs represent the mean±SEM. *p<0.05, ** p<0.01 (n=6-8 mice/group).

FIG. 17: Effects of PM_{2.5} on the lungs of COPD mice. Severe inflammatory changes were observed in the lung parenchyma of the elastase/LPS-exposed mice after intratracheal injection of 125 μg of PM_{2.5}. Panels A, B and C show mononuclear cell infiltration of the lung parenchyma. Panel D illustrates aggregations of PM_{2.5} particles inside inflammatory macrophage-like cells (Black Arrows). PM_{2.5} induced Goblet Cell Metaplasia in elastase/LPS treated mice. This figure shows lung sections of different study groups of mice that were stained with periodic acid-Schiff (PAS) reagent. Panel E and F show normal airway epithelium of the control group. Panel G shows moderate goblet cells metaplasia in Elastase/LPS treated mice. Panels H and I are and show goblet cell metaplasia with abundant PAS-positive cells in the airways of PM_{2.5} challenged mice, and Panel J shows the airway epithelium in CMC 2.24 treated group.

FIG. 18: Effect of CMC 2.24 on MMP-9 and MMP-2 activities in the BALF supernatants of COPD mice exposed to PM_{2.5}. Gelatin zymographic analysis of the BALF recovered from the airways of COPD mice exposed to PM_{2.5} and COPD mice exposed to PM_{2.5} and treated with CMC 2.24 daily for 7 days. Panels A and B show significantly increased activity of MMP-9 in BALF supernatants from COPD-mice compared to control mice (P<0.01) and a many-fold increase in the COPD-mice exposed to PM_{2.5}. MMP-9 activity was significantly inhibited in mice exposed to PM_{2.5} when treated with CMC 2.24 (P<0.05). Panels C and D demonstrate the significant increase in MMP-2 activity in PM_{2.5}-exposed mice (P<0.05). The levels of MMP-9 and MMP-2 remain normal in COPD-mice exposed to PM_{2.5} when treated with CMC 2.24. These results demonstrate that CMC 2.24 treatment essentially reduces these excessive levels back down to healthy “control” levels under circumstances where MMP-2, and 9 levels had been increased by COPD or by COPD exacerbated by a PM_{2.5} challenge. Graphs represent the mean±SEM. *p<0.05, ** p<0.01 (n=6-8 mice/group).

FIG. 19: Effect of CMC 2.24 on MMP-12 activity in BALF supernatants of COPD mice exposed to PM_{2.5}. In Panels A casein zymographic analysis demonstrates the significant increase in MMP-12 activity (P<0.01) in COPD-mice compared to control mice (Panel B). This increase was significantly inhibited in mice exposed to PM_{2.5} when treated with CMC 2.24 (P<0.05). Graphs represent the mean±SEM. *p<0.05, ** p<0.01 (n=6-8 mice/group).

FIG. 20: Mice exposed to 125 μg of PM_{2.5} showed marked and significant influx of inflammatory cells in both the lung tissue and BAL fluid up to seven days post exposure.

FIG. 21: BALF cell content changes in response to administration of both PM_{2.5} and CMC 2.24 in COPD-mice. Cytological analysis of BALF shows a significant increase in the percentage of neutrophils in exacerbated COPD mice exposed to PM_{2.5} compared with COPD mouse controls. However, COPD mice exposed to PM_{2.5} and treated with CMC 2.24 were protected from this increase in acute inflammatory cells. Graphs represent the mean±SEM. *p<0.05, * p<0.01 (n=5 mice/group).

FIG. 22: Effect of CMC 2.24 on inflammatory cytokines, TNF-α and IL-6 levels in the BALF supernatants of COPD-mice exposed to PM_{2.5}. The levels of TNF-α and IL-6 in BAL fluid were determined by ELISA. The level of TNF-α increases significantly in PM_{2.5} challenged mice (P<0.05) but decreases substantially (P<0.05) when these mice receive CMC 2.24 (Panel A). The level of IL-6 also increased significantly in PM2.5 challenged mice (P<0.01) and decreased greatly in PM_{2.5}—challenged mice that were treated with CMC 2.24 (P<0.05: Panel B). Control-mice were treated with saline, whereas COPD-mice were generated by PPE (porcine pancreatic elastase)+LPS treatment. Graphs represent the mean±SEM. *p<0.05, ** p<0.01 (n=5-7 mice/group).

FIG. 23: Effect of CMC 2.24 on the levels of 8-Isoprostane as a marker for oxidative stress. The levels of 8-Isoprostane were measured in BALF using ELISA analysis. The results showed significant increase in the levels of 8-Isoprostane in the BALF of PM_{2.5} challenged mice (p<0.05). However, the levels of 8-Isoprostane in the BALF were decreased substantially after CMC 2.24 treatment (p<0.01). Graphs represent the mean±SEM. *p<0.05, ** p<0.01 (n=5-7 mice/group).

FIG. 24: Effect of CMC 2.24 on the levels of Phosphorylated IκB-α. We measured phosphorylated IκB-α using western blot. IκB-α activates NF-κB and consequently modulate the transcription of genes controlling inflammatory response. Higher levels of IκB-α are associated with inflammation. We found significant increase in the level of IκB-α in PM_{2.5} challenged mice (P<0.05) and significant decrease in PM_{2.5} challenged mice when treated with CMC 2.24 (P<0.05). Graphs represent the mean±SEM. *p<0.05, ** p<0.01 (n=3-4 mice/group).

FIG. 25: Apoptotic cells in the lungs of elastase/LPS-treated mice, and PM_{2.5}-challenged mice with or without CMC 2.24 treatment. Apoptotic cell levels were determined by TUNEL assay in the lung tissues of elastase/LPS-treated mice (Panel B), and PM_{2.5}-challenged mice with (Panel C) and with CMC 2.24 treatment (Panel D). The cells with brown nuclei are apoptotic (arrows). They were quantified by the high-power field procedure as described in the methods section. The results show that there are large numbers of apoptotic cells in the elastase/LPS-treated mice that increase significantly when these mice are challenged with PM_{2.5}. By contrast the group of mice treated with CMC 2.24 showed a significant reduction in the number of apoptotic cells present. Graphs represent the mean±SEM. *p<0.05, ** p<0.01 (n=3-5 mice/group).

FIG. 26: Effect of CMC 2.24 on the levels of Bcl-2 as a marker for apoptosis Bcl-2 expression as a negative marker for apoptosis was measure using western blot. Data shown demonstrate significantly lower levels of Bcl-2 expression in COPD mice in comparison to control mice (p<0.05). It also show that the administration of CMC 2.24 leads to significantly higher levels of Bcl-2 expression (p<0.05). Graphs represent the mean±SEM. *p<0.05, ** p<0.01 (n=3-4 mice/group).

FIG. 27: The effects of different concentrations of CMC 2.24 on cell viability in lung epithelial cell line (A549) and primary alveolar macrophages. A549 cells (A) and primary macrophages (C) were treated with different concentrations of CMC 2.24 for 24 h. A549 cells (B) and primary alveolar macrophages (D) were treated with different concentrations of CMC2.24 for 0.5 h prior to 100 μg/ml PM2.5 treatment. After 24 h of PM2.5 treatment cell viability was assessed by CCK-8 kit. Each column represents mean±SEM (experimental number n=6). ***p<0.001 versus the control group; ^{##}p<0.01 and ^{###}p<0.001 versus the PM2.5 group. FIG. 28: Effects of CMC2.24 on cell death of PM2.5-treated A549 cells. Cells were pre-treated with a range of concentrations of CMC2.24 for 0.5 h prior to treatment with 100 μg/ml PM2.5. After 24 h of PM2.5 treatment, dead cells were examined using trypan blue staining. Cells were observed under a phase-contrast microscope (magnification, ×200). Dead cells were stained with blue (Panel A). The percentage of dead cells/total cells per field was analyzed and compared among groups (Panel B). Each column represents mean±SEM (experimental number n=5). ***p<0.001 versus the control group; ^{#}p<0.05, ^{##}p<0.01 and ^{###}p<0.001 versus the PM2.5 group.

FIG. 28: Effects of CMC2.24 on cell death of PM2.5-treated A549 cells. Cells were pre-treated with a range of concentrations of CMC2.24 for 0.5 h prior to treatment with 100 μg/ml PM2.5. After 24 h of PM2.5 treatment, dead cells were examined using trypan blue staining. Cells were observed under a phase-contrast microscope (magnification, ×200). Dead cells were stained with blue (Panel A). The percentage of dead cells/total cells per field was analyzed and compared among groups (Panel B). Each column represents mean±SEM (experimental number n=5). ***p<0.001 versus the control group; ^{#}p<0.05, ^{##}p<0.01 and ^{###}p<0.001 versus the PM2.5 group.

FIG. 29: Effects of CMC 2.24 on the NF-κB p65 expression and nuclear translocation in A549 cells. Cells were pre-treated with different concentrations of CMC2.24 for 0.5 h prior to treatment with 100 μg/ml of PM2.5. After 24 h of PM2.5 treatment NF-κB p65 expression and nuclear translocation in A549 cells were analyzed using immunohistochemical method with specific anti-p65 antibody (A). The nuclei of the corresponding cells were stained by haematoxylin. Original magnification ×400. The ratio of NF-κB p65 nuclear positive cells/total cells was analyzed (B) and each column represents mean±SEM (experimental number n=5). ***p<0.001 versus the control group; ^{###}p<0.001 versus the PM2.5 group; ^{#}p<0.05 versus the PM2.5+CMC2.24 (10 μM) group.

FIG. 30: A schematic diagram of the functional mechanisms of CMC 2.24 effects. PM2.5 exposure or other inflammatory mediators induced NF-κB signaling activation in lung epithelial cells and alveolar macrophages; then overactivated NF-κB signaling pathway caused cell apoptotic pathway and lead to cell death. CMC 2.24 could inhibit PM2.5-induced ikb kinase activity and involve blockade of IκB degradation and the nuclear translocation of NF-κB p65. Therefore, CMC 2.24 could attenuate the transcription and expression of various inflammatory mediators.

FIG. 31: Histological analysis of the lungs from CMC 2.24-treated and untreated emphysematous SP-D KO mice. Formalin-fixed, paraffin-embedded lung tissues from emphysematous SP-D KO mice with and without CMC 2.24 treatment were stained with hematoxylin and eosin (H&E). Panel A shows lung section with widening of the airspaces from emphysematous SP-D KO mice (control). Panel B shows health status lung in CMC 2.24-treated SP-D KO mice. (n=3 mice/group).

FIG. 32: Total cell count in the BALF obtained from control and CMC 2.24-treated mice. Total cells from the BALF of control (vehicle treatment) and CMC 2.24-treated mice were counted using a hemocytometer method. The data demonstrate significantly higher cell count in the control mice than CMC 2.24-treated mice (p<0.05). The data are the number of cells in the BAL fluid per mouse. Graphs represent the mean±SEM. *p<0.05 (n=4 mice/group).

FIG. 33: Different phenotypes of alveolar macrophages between CMC 2.24-treated and control mice. Total BALF cells of CMC 2.24-treated and control mice were prepared and mounted on the slides by cytospin centrifugation method and then stained with hematoxylin and eosin (H&E). Cell morphology were examined by a light microscope. The data show ballooned and vacuolated macrophages in control mice but health and normal alveolar macrophages in CMC 2.24-treated mice.

FIG. 34: Effects of CMC 2.24 on MMPs 2 and 9 activities in the BALF of emphysematous mice. The samples of BALF were prepared from CMC 2.24-treated emphysematous mice and control mice. The levels of MMPs 2 and 9 activity were examined using gelatin zymographic analysis. Panels A and B show significant lower level of MMP 9 in the BALF of CMC 2.24-treated mice than that in the BALF of control mice (p<0.05). Similarly, Panels C and D show decreased level of MMP 2 activity in the BALF of CMC 2.24-treated mice compared to control mice (p<0.05). These results demonstrate that CMC 2.24 treatment essentially reduces these excessive MMP levels back down to healthy levels. Graphs represent the mean±SEM. *p<0.05, ** p<0.01 (n=4 mice/group).

FIG. 35: The levels of bioluminescence in infected SP-B-C and SP-B-T mice. The levels of bioluminescent signal which represents bacterial number in the lung of infected mice were measured at several time points from 0 to 48 hours by in vivo imaging system. The results showed that infected SP-B-C mice exhibit higher level of bioluminescence than infected SP-B-T mice from 24 h to 48 h after infection. (A) The representative image of bioluminescence at each time point in infected SP-B-C and SP-B-T mice; (B) Comparisons of the bioluminescent level at each time point between infected SP-B-C and SP-B-T mice. *p<0.05, **p<0.01 (n=15 mice/group)

FIG. 36: The levels of bioluminescence in the lung of male and female mice. The levels of bioluminescence were determined in infected male and female mice by in vivo image system. The results indicated the timing of bacterial growth peak differs between male and female mice. The level of bioluminescence in infected male mice reached highest at 12 h after infection and then turned to decrease while the peak of bioluminescent level in infected female mice are at 28 h and 32 h after infection. (A) The representative image of bioluminescence at each time point in infected SP-B-C and SP-B-T mice; (B) Comparisons of the bioluminescent level at each time point between infected SP-B-C and SP-B-T mice. *p<0.05, ** p<0.01 (n=15 mice/group)

FIG. 37: The levels of bioluminescence in the mice with pneumonia v.s. pneumonia with CMC2.24 treatment. Infected SP-B-C and SP-B-T mice were administered a daily dose of CMC2.24 (50 mg/kg) or vehicle by gavage. The levels of bioluminescence were measured for 48 h after infection by in vivo image system. The levels of bioluminescence in the CMC2.24-treated group (Pneu+CMC) were lower compared to the control group (Pneu) for both infected SP-B-C and SP-B-T mice starting at 24 h and 28 h after infection, respectively. (A) The representative image of bioluminescence at 28 h in infected SP-B-C and SP-B-T mice. Comparisons of the bioluminescent level at each time point in infected SP-B-C(B) and SP-B-T (C) mice with and without CMC2.24 treatment. *p<0.05, **p<0.01 (n=15 mice/group)

FIG. 38: Histology of the lung in infected SP-B-C and SP-B-T mice with and without CMC2.24 treatment. The histopathology of lung tissues were analyzed in three groups, i.e. Sham, pneumonia (Pneu), and pneumonia plus CMC2.24 treatment (pneu+CMC) of SP-B-C and SP-B-T mice. Histological sections from three groups were stained with H/E (A) and the histopathological score of lung injury was assessed (B). Lung histology shows inflammatory cells in alveoli and interstitial membrane, proteinaceous debris, and wider alveolar wall in the lung tissues of infected mice but not in Sham mice. Compared to infected SP-B-T mice with or without CMC2.24, the lung injury score is higher in infected SP-B-C mice with or without CMC2.24, respectively. The score of lung injury is lower in the CMC2.24-treated SP-B-C and SP-B-T mice compared to pneumonia SP-B-C and SP-B-T mice, respectively. Naïve control=Sham, pneumonia—Pneu, pneumonia with CMC2.24=Pneu+CMC; Bar=50 μm; Graphs represent the mean±SEM. *p<0.05, ** p<0.01 (n=8 mice/group).

FIG. 39: Apoptotic cells in the lung of infected SP-B-C and SP-B-T mice with and without CMC2.24 treatment. Apoptotic cells were examined with TUNEL assay in the lung tissues of infected SP-B-C and SP-B-T mice treated with CMC2.24 (Pneu+CMC), vehicle (Pneu), or naïve control (Sham) (A). The cells with brown nucleus are apoptotic (arrows). Apoptotic cells were quantified per high-power field as described in the methods (B). The results showed that there are significant amounts of apoptotic cells in the infected mice but not in sham mice. The number of apoptotic cells in the lung tissues of infected SP-B-C mice (Pneu, Pneu+CMC) was larger compared to infected SP-B-T mice (Pneu, Pneu+CMC), respectively. After CMC2.24 treatment, decreased apoptotic cells were observed in both infected SP-B-C and SP-B-T mice. naïve control—Sham, pneumonia—Pneu, pneumonia plus CMC2.24 treatment—Pneu+CMC; Bar-50 μm; Graphs represent the mean±SEM. *p<0.05, ** p<0.01 (n=8 mice/group).

FIG. 40: The levels of apoptotic and anti-apoptotic biomarkers in the lung of infected SP-B-C and SP-B-T mice. The levels of apoptosis (Caspase-3) (Panel A) and anti-apoptosis (Bcl-2) (Panel B) biomarkers in the lung tissues were analyzed by Western blotting analysis, and quantified by densitometry. The data were normalized by the level of 5-actin. The level of caspase-3 increased significantly in the infected mice (Pneu, Pneu+CMC) compared to Sham (p<0.01). The levels of caspase-3 in infected SP-B-C mice (Pneu and Pneu+CMC) are higher (p<0.01) than that of infected SP-B-T mice (Pneu and Pneu+CMC), respectively. With treatment of CMC2.24, the levels of acspase-3 decreased in both infected SP-B-C and SP-B-T mice. For anti-apoptosis biomarker (Bcl-2), the level of Bcl-2 is lower in the infected mice (Pneu, Pneu+CMC) compared to Sham (p<0.01). With treatment of CMC2.24, the level of Bcl-2 increased significantly in both infected SP-B-C and SP-B-T mice. Naïve control—Sham, pneumonia—Pneu, pneumonia plus CMC2.24 treatment—Pneu+CMC; Bar-50 μm; Graphs represent the mean±SEM. *p<0.05, ** p<0.01 (n=8 mice/group).

FIG. 41: Inflammatory cells in BALF from infected SP-B-C and SP-B-T mice with and without CMC2.24 treatment. Samples of BALF were prepared from three groups (Sham, Pneu, and Pneu+CMC) of SP-B-C and SP-B-T mice. The cells in each BALF samples were mounted on slide by cytospin centrifuge method. The Slides from three groups were stained with using the Hema-3 Stain Kit (A). With light microscopy, neutrophils (PMN) and macrophages/monocytes per slide were analyzed and quantified at ×400 magnification. The number of neutrophils and macrophages/monocytes were compared among Sham, Pneu, Pneu+CMC groups. In the BALF of Sham group more than 98% of cells are macrophages but no neutrophils. The number of neutrophils increased significantly in the BALF from infected SP-B-C and SP-B-T mice compared to Sham mice. The numbers of neutrophils and macrophages are larger in the BALF from infected SP-B-C mice compared to infected SP-B-T mice. With treatment of CMC2.24, the number of neutrophils and macrophages in the BALF from both SP-B-C and SP-B-T decreased significantly. Naïve control—Sham, pneumonia—Pneu, pneumonia plus CMC2.24 treatment—Pneu+CMC; Bar-50 μm; Graphs represent the mean±SEM. *p<0.05, ** p<0.01 (n=8 mice/group).

FIG. 42: Expression of NF-κB p65/p-IκB in the lung of infected SP-B-C and SP-B-T mice. The levels of inflammatory NF-κB p65 protein in the lung tissues of infected mice and Sham were examined by Western blotting analysis and then quantified by densitometry. The data were normalized by the levels of β-actin. Panels A and B show the bolts and quantitative results, respectively. Compared to Sham group, infected SP-B-C and SP-B-T mice (Pneu and Pneu+CMC) have higher levels of NF-κB p65 expression. CMC2.24 treatment decreased significantly the levels of NF-κB p65 expression in the lung tissues of infected mice. Naïve control—Sham, pneumonia—Pneu, pneumonia plus CMC2.24 treatment—Pneu+CMC; Graphs represent the mean±SEM. *p<0.05, ** p<0.01 (n=8 mice/group).

FIG. 43: The levels of secreted SP-B in the BALF of infected SP-B-C and SP-B-T mice. Samples of BALF were obtained from three mouse groups (Sham, Pneu, and Pneu+CMC) of SP-B-C and SP-B-T mice. The level of total proteins in the BALF of three groups were determined using the BCA micro assay kit. Five micrograms of each BALF sample were used for analysis of SP-B by Western blotting analysis as described in the method. Panels A and B show the bolts and quantitative results, respectively. The results showed that the levels of SP-B in the BALF from infected mice (Pneu and Pneu+CMC) decreased significantly compared to Sham mice. The order of the levels of SP-B in the BAL is as Sham>Pneu+CMC>Pneu. Naïve control—Sham, pneumonia—Pneu, pneumonia plus CMC2.24 treatment—Pneu+CMC; Graphs represent the mean±SEM. *p<0.05, ** p<0.01 (n=8 mice/group).

FIG. 44: MMPs activity in the BALF of infected infected SP-B-C and SP-B-T mice. The activities of MMP-2, -9, -12 were examined in the BALF of three groups (Sham, Pneu, and Pneu+CMC) of SP-B-C and SP-B-T mice by gel zymography as described in the method. Panels A and B show the zymographic gels bolts and quantitative results of MMP-2, -9, -12 activities, respectively. No detectable levels of MMP-2, -9, -12 activities were observed in Sham mice. Significant activities of MMP-2, -9, -12 were determined in the BALF of infected mice with infected SP-B-C higher than infected SP-B-T mice. With treatment of CMC2.24, the levels of activities of MMP-2, -9, -12 decreased significantly (p<0.05) in both infected SP-B-C and SP-B-T mice. Naïve control—Sham, pneumonia—Pneu, pneumonia plus CMC2.24 treatment=Pneu+CMC; Graphs represent the mean±SEM. *p<0.05, ** p<0.01 (n=8 mice/group).

FIG. 45: Ratio of short-term inflammatory cytokine (IL-1β), relative to the resolvin, lipoxin A4, secreted by peritoneal macrophages in cell culture (a). Concentration in conditioned media of cultured macrophages from 3 different groups of rats (b). LXA4 Concentration in conditioned media of cultured macrophages from 3 different groups of rats (c).

FIG. 46: Ratio of short-term inflammatory cytokine (IL-1β)/resolvin in macrophages in cell culture (a). IL-1β concentration in conditioned media of macrophages in cell culture (b). Lipoxin A4 concentration in conditioned media of macrophages in cell culture (c).

FIG. 47: Ratio of long-term inflammatory cytokine (IL-6)/resolvin in macrophages in cell culture (a). IL-6 concentration in conditioned media of macrophages in cell culture (b). Lipoxin A4 concentration in conditioned media of macrophages in cell culture (c).

### DETAILED DESCRIPTION OF THE INVENTION

The present invention provides a method of treating a subject afflicted with a disease or condition comprising administering to the subject an amount of a compound having the structure:

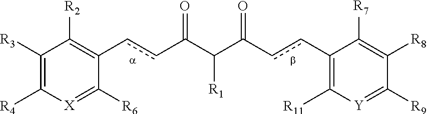

wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH) NR₁₂R₁₃ —SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
   wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
   R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

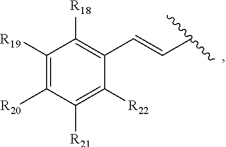

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO—R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂, are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₉R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R₁ is H, then R₃, R₄, R₅, R₈, R₉, or R₁₀, is halogen, —NO₂, —CN, —NR₂₉R₂₉, —NHR₂₈R₂₉^{x}, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt or ester thereof, so as to thereby treat the subject, wherein the disease or condition is selected from chronic inflammation, chronic inflammatory disease, rheumatoid arthritis, psoriatic arthritis, osteoarthritis, periodontitis, inflammatory bowel disease, irritable bowel syndrome, psoriasis, ankylosing spondylitis, Sjogren's syndrome, multiple sclerosis, ulcerative colitis, Crohn's disease, systemic lupus erythematosus, lupus nephritis, psoriasis, celiac disease, vasculitis, atherosclerosis, cystic fibrosis, asthma, chronic obstructive pulmonary disease (COPD), bacterial pneumonia, pulmonary bacterial pneumonia, chronic bronchitis, emphysema, chronic and acute lung inflammatory disease, pneumonia, asthma, acute lung injury, lung cancer, diabetes and pulmonary impairment.

In some embodiments of the above method, the disease or condition is selected from chronic inflammation, chronic inflammatory disease, psoriasis, psoriatic arthritis, ankylosing spondylitis, Sjogren's syndrome, ulcerative colitis, Crohn's disease, systemic lupus erythematosus, lupus nephritis, psoriasis, celiac disease, vasculitis, cystic fibrosis, asthma, chronic obstructive pulmonary disease (COPD), bacterial pneumonia, pulmonary bacterial pneumonia, chronic bronchitis, chronic and acute lung inflammatory disease, pneumonia, asthma, acute lung injury, lung cancer and pulmonary impairment.

In some embodiments of the above method, the disease or condition is selected from cystic fibrosis, asthma, chronic obstructive pulmonary disease (COPD), bacterial pneumonia, pulmonary bacterial pneumonia, chronic bronchitis, chronic and acute lung inflammatory disease, pneumonia, asthma, acute lung injury, lung cancer and pulmonary impairment.

In some embodiments of the above method, the disease or condition is chronic or acute lung inflammatory disease.

In some embodiments of the above method, the chronic or acute lung inflammatory disease is chronic obstructive pulmonary disease (COPD), pneumonia, asthma, acute lung injury, lung cancer or pulmonary impairment.

In some embodiments of the above method, the chronic or acute lung inflammatory disease is COPD exacerbation induced by exposure to an environmental factor.

In some embodiments of the above method, the environmental factor is a particulate matter 2.5 microns or smaller.

In some embodiments of the above method, the chronic or acute lung inflammatory disease is chronic bronchitis or emphysema.

In some embodiments of the above method, the chronic or acute lung inflammatory disease is chronic bronchitis or emphysema.

In some embodiments of the above method, the chronic or acute lung inflammatory disease is bacterial pneumonia.

In some embodiments, the method wherein the subject is afflicted with acute disease exacerbations triggered by air pollutants.

In some embodiments of the above method, the subject is normoglycemic.

In some embodiments of the above method, the subject is hyperglycemic.

In some embodiments of the above method, the treating comprises inducing production of the one or more lipoxins in the subject.

In some embodiments of the above method, the one or more lipoxins are selected from lipoxin A4, 15-epi-LXA4 and lipoxin B4.

In some embodiments of the above method, the method further comprising inducing production of one or more resolvins in the subject.

In some embodiments of the above method, the one or more resolvins are selected from RvE1, RvE2, RvE3, RvD1, RvD2, RvD3, RvD4 and RvD5.

In some embodiments of the above method, the method further comprising increasing production of one or more protectins in the subject.

In some embodiments of the above method, wherein wherein the one or more protectins is PD1-NPD1.

In some embodiments of the above method, the method further comprising increasing production of one or more maresins in the subject.

In some embodiments of the above method, the one or more maresins is MaR1.

In some embodiments of the above method, the method further comprising inducing production of one or more anti-inflammatory cytokines in the subject.

In some embodiments of the above method, wherein the one or more anti-inflammatory cytokines are selected from IL-10 and TGF-β.

In some embodiments of the above method, the method further comprising reducing production of one or more pro-inflammatory cytokines in the subject.

In some embodiments of the above method, wherein the one or more pro-inflammatory cytokines are selected from IL-6, IL-□ and TNF-α.

In some embodiments of the above method, the method further comprising increasing production of one or more resolvins in the subject, one or more protectins in the subject, one or more maresins in the subject, one or more maresins in the subject and/or one or more anti-inflammatory cytokines in the subject.

In some embodiments of the above method, the method comprising increasing production of one or more lipoxins in the subject and reducing production of one or more pro-inflammatory cytokines in the subject.

The present invention provides a method of increasing production of one or more lipoxins in a subject in need thereof comprising administering to the subject an amount of a compound having the structure:

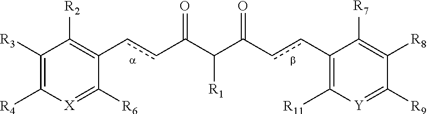

wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH) NR₁₂R₁₃ —SR₁₂, —SO₂R₁₃, —COR₁₄, —CSR₁₄, —C(═NR₁₂)R₁₄, —C(═NR₁₂) NR₁₃R₁₄, —SOR₁₂, —SONR₁₂R₁₃, —SO₂NR₁₂R₁₃, —P(O)R₁₂, —PH(═O)OR₁₂ —P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

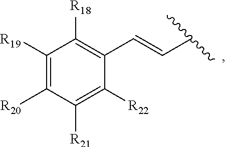

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R₁ is H, then R₃, R₄, R₅, R₈, R₉, or R₁₀, is halogen, —NO₂, —CN, —NR₂₈R₂₉, —NHR₂₈R₂₉, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt or ester thereof, so as to thereby increase production of the one or more lipoxins in the subject.

In some embodiments, the one or more lipoxins are selected from lipoxin A4, 15-epi-LXA4 and lipoxin B4.

In some embodiments, the methods further comprising increasing production of one or more resolvins in the subject.

In some embodiments, the one or more resolvins are selected from RvE1, RvE2, RvE3, RvD1, RvD2, RvD3, RvD4 and RvD5.

In some embodiments, the method further comprising increasing production of one or more protectins in the subject.

In some embodiments, the one or more protectins is PD1-NPD1.

In some embodiments, the method further comprising increasing production of one or more maresins in the subject.

In some embodiments, wherein the one or more maresins is MaR1.

In some embodiments, the method further comprising increasing production of one or more anti-inflammatory cytokines in the subject.

In some embodiments, the one or more cytokines are selected from IL-10 and TGF-β.

In some embodiments, the method further comprising decreasing production of one or more pro-inflammatory cytokines in the subject.

In some embodiments, the one or more proinflammatory cytokines are selected from IL-6 and IL-β.

In some embodiments, the one or more proinflammatory cytokines are selected from TNF-α, IL-6 and IL-β.

In some embodiments, the method wherein the subject is afflicted with a disease or condition associated with decreased levels of one or more lipoxins.

In some embodiments, the method wherein the subject is afflicted with chronic inflammation or a chronic inflammatory disease.

In some embodiments, the method wherein the subject is afflicted with acute disease exacerbations triggered by air pollutants.

In some embodiments, the method wherein subject is afflicted with rheumatoid arthritis, osteoarthritis, psoriatic arthritis, periodontitis, inflammatory bowel disease, irritable bowel syndrome, psoriasis, ankylosing spondylitis, Sjogren's syndrome, multiple sclerosis, ulcerative colitis and Crohn's disease, systemic lupus erythematosus, lupus nephritis, psoriasis, celiac disease, vasculitis, atherosclerosis, cystic fibrosis, asthma, or chronic obstructive pulmonary disease (COPD).

In some embodiments of the above method, the subject is afflicted with chronic inflammation, chronic inflammatory disease, rheumatoid arthritis, psoriatic arthritis, osteoarthritis, periodontitis, inflammatory bowel disease, irritable bowel syndrome, psoriasis, ankylosing spondylitis, Sjogren's syndrome, multiple sclerosis, ulcerative colitis, Crohn's disease, systemic lupus erythematosus, lupus nephritis, psoriasis, celiac disease, vasculitis, atherosclerosis, cystic fibrosis, asthma, chronic obstructive pulmonary disease (COPD), bacterial pneumonia, pulmonary bacterial pneumonia, chronic bronchitis, emphysema, chronic and acute lung inflammatory disease, pneumonia, asthma, acute lung injury, lung cancer, diabetes or pulmonary impairment.

In some embodiments of the above method, the subject is afflicted with chronic inflammation, chronic inflammatory disease, psoriasis, psoriatic arthritis, ankylosing spondylitis, Sjogren's syndrome, ulcerative colitis, Crohn's disease, systemic lupus erythematosus, lupus nephritis, psoriasis, celiac disease, vasculitis, cystic fibrosis, asthma, chronic obstructive pulmonary disease (COPD), bacterial pneumonia, pulmonary bacterial pneumonia, chronic bronchitis, chronic and acute lung inflammatory disease, pneumonia, asthma, acute lung injury, lung cancer or pulmonary impairment.

In some embodiments of the above method, the subject is afflicted with cystic fibrosis, asthma, chronic obstructive pulmonary disease (COPD), bacterial pneumonia, pulmonary bacterial pneumonia, chronic bronchitis, chronic and acute lung inflammatory disease, pneumonia, asthma, acute lung injury, lung cancer or pulmonary impairment.

In some embodiments of the above method, the subject is afflicted with chronic or acute lung inflammatory disease.

In some embodiments of the above method, the subject is afflicted with the chronic: or acute lung inflammatory disease is chronic obstructive pulmonary disease (COPD), pneumonia, asthma, acute lung injury, lung cancer or pulmonary impairment.

In some embodiments of the above method, the subject is afflicted with the chronic: or acute lung inflammatory disease is COPD exacerbation induced by exposure to an environmental factor.

In some embodiments of the above method, the environmental factor is a particulate matter 2.5 microns or smaller.

In some embodiments of the above method, the chronic or acute lung inflammatory disease is chronic bronchitis or emphysema.

In some embodiments of the above method, the chronic or acute lung inflammatory disease is chronic bronchitis or emphysema.

In some embodiments of the above method, the chronic or acute lung inflammatory disease is bacterial pneumonia.

In some embodiments of the above method, the subject is normoglycemic.

In some embodiments of the above method, the subject is hyperglycemic.

The present invention also provides a method of treating a subject afflicted with a respiratory disease, a dermatologic disease, a musculoskeletal disease, a gastrointestinal disease, a cardiovascular disease, a neurodegenerative disease, an ophthalmic disease, a oral health disease or a cancer.

The present invention also provides a method of treating a subject afflicted with a disease associated with decreased levels of one or more lipoxins comprising inducing production of the one or more lipoxins in the subject by administering to the subject an amount of a compound having the structure:

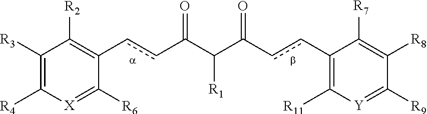

wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃ —SR₁₂, —SO₂R₁₃, —COR₁₄, —CSR₁₄, —C(═NR₁₂)R₁₄, —C(═NR₁₂) NR₁₃R₁₄, —SOR₁₂, —SONR₁₂R₁₃, —SO₂NR₁₂R₁₃, —P(O)R₁₂, —PH(═O)OR₁₂ —P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

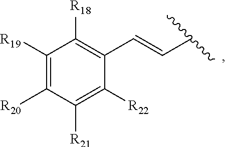

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
   R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
      wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
   R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
      wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀alkenyl, C₂₋₁₀ alkynyl, or —C(═O)-heterocyclyl; and
wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₉, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt or ester thereof, so as to thereby treat the subject afflicted with the disease.

In some embodiments, the one or more lipoxins are selected from lipoxin A4, 15-epi-LXA4 and lipoxin B4.

In some embodiments, the method further comprising inducing production of one or more resolvins in the subject.

In some embodiments, the one or more resolvins are selected from RvE1, RvE2, RvE3, RvD1, RvD2, RvD3, RvD4 and RvD5.

In some embodiments, the method further comprising increasing production of one or more protectins in the subject.

In some embodiments, the one or more protectins is PD1-NPD1.

In some embodiments, the method further comprising increasing production of one or more maresins in the subject.

In some embodiments, the one or more maresins is MaR1.

In some embodiments, the method further comprising inducing production of one or more anti-inflammatory cytokines in the subject.

In some embodiments, the one or more anti-inflammatory cytokines are selected from IL-10 and TGF-β.

In some embodiments, the method further comprising reducing production of one or more pro-inflammatory cytokines in the subject.

In some embodiments, the one or more pro-inflammatory cytokines are selected from IL-6, IL-β and TNF-α.

In some embodiments of the above method, the method further comprising increasing production of one or more resolvins in the subject, one or more protectins in the subject, one or more maresins in the subject, one or more maresins in the subject and/or one or more anti-inflammatory cytokines in the subject.

In some embodiments, the method wherein the disease associated with decreased levels of one or more lipoxins is chronic inflammation or a chronic: inflammatory disease.

In some embodiments, the method wherein disease associated with decreased levels of one or more lipoxins is rheumatoid arthritis, osteoarthritis, psoriatic arthritis, periodontitis, inflammatory bowel disease, irritable bowel syndrome, psoriasis, ankylosing spondylitis, Sjogren's syndrome, multiple sclerosis, ulcerative colitis and Crohn's disease, systemic lupus erythematosus, lupus nephritis, psoriasis, celiac disease, vasculitis, atherosclerosis, cystic fibrosis, asthma, and chronic obstructive pulmonary disease (COPD).

In some embodiments, the method wherein the disease associated with decreased levels of one or more lipoxins is a respiratory disease.

In some embodiments of any of the disclosed methods, the respiratory disease is selected from acute respiratory distress syndrome, chronic obstructive pulmonary disease, asthma, emphysema and idiopathic pulmonary fibrosis.

In some embodiments, the method wherein disease associated with decreased levels of one or more lipoxins is a dermatologic disease.

In some embodiments of any of the disclosed methods, the dermatologic disease is selected from psoriasis, acne, and rosacea.

In some embodiments, the method wherein disease associated with decreased levels of one or more lipoxins is a musculoskeletal disease.

In some embodiments of any of the disclosed methods, the musculoskeletal disease is selected from rheumatoid arthritis, osteoarthritis and osteoporosis.

In some embodiments, the method wherein disease associated with decreased levels of one or more lipoxins is a gastrointestinal disease.

In some embodiments of any of the disclosed methods, the gastrointestinal disease is selected from inflammatory bowel disease, ulcerative colitis, Crohn's disease, hemorrhoids and piles.

In some embodiments, the method wherein disease associated with decreased levels of one or more lipoxins is a cardiovascular disease.

In some embodiments of any of the disclosed methods, the cardiovascular disease is selected from myocardial infarction, atherosclerosis, hypertension, acute coronary syndromes and aortic aneurisms.

In some embodiments, the method wherein disease associated with decreased levels of one or more lipoxins is a neurodegenerative disease.

In some embodiments of any of the disclosed methods, the neurodegenerative disease is selected from multiple sclerosis, Parkinson's disease, alzheimer's disease, amyotrophic lateral sclerosis and huntington's disease.

In some embodiments, the method wherein disease associated with decreased levels of one or more lipoxins is an ophthalmic disease.

In some embodiments of any of the disclosed methods, the ophthalmic disease is selected from sterile corneal ulcers, retinopathy, glaucoma, macular degeneration, wet cataract, and dry cataract.

In some embodiments, the method wherein disease associated with decreased levels of one or more lipoxins is an oral health disease.

In some embodiments of any of the disclosed methods, the oral health disease is selected from pemphigoid and oral mucositis.

In some embodiments, the method wherein disease associated with decreased levels of one or more lipoxins is cancer.

In some embodiments of any of the disclosed methods, the cancer is selected from liver cancer, bone cancer, colon cancer, pancreatic cancer, lung cancer and breast cancer.

In some embodiments of the above method, the subject is afflicted with chronic inflammation, chronic inflammatory disease, rheumatoid arthritis, psoriatic arthritis, osteoarthritis, periodontitis, inflammatory bowel disease, irritable bowel syndrome, psoriasis, ankylosing spondylitis, Sjogren's syndrome, multiple sclerosis, ulcerative colitis, Crohn's disease, systemic lupus erythematosus, lupus nephritis, psoriasis, celiac disease, vasculitis, atherosclerosis, cystic fibrosis, asthma, chronic obstructive pulmonary disease (COPD), bacterial pneumonia, pulmonary bacterial pneumonia, chronic bronchitis, emphysema, chronic and acute lung inflammatory disease, pneumonia, asthma, acute lung injury, lung cancer, diabetes or pulmonary impairment.

In some embodiments of the above method, the subject is afflicted with chronic inflammation, chronic inflammatory disease, psoriasis, psoriatic arthritis, ankylosing spondylitis, Sjogren's syndrome, ulcerative colitis, Crohn's disease, systemic lupus erythematosus, lupus nephritis, psoriasis, celiac disease, vasculitis, cystic fibrosis, asthma, chronic obstructive pulmonary disease (COPD), bacterial pneumonia, pulmonary bacterial pneumonia, chronic bronchitis, chronic and acute lung inflammatory disease, pneumonia, asthma, acute lung injury, lung cancer or pulmonary impairment.

In some embodiments of the above method, the subject is afflicted with cystic fibrosis, asthma, chronic obstructive pulmonary disease (COPD), bacterial pneumonia, pulmonary bacterial pneumonia, chronic bronchitis, chronic and acute lung inflammatory disease, pneumonia, asthma, acute lung injury, lung cancer or pulmonary impairment.

In some embodiments of the above method, the subject is afflicted with chronic or acute lung inflammatory disease.

In some embodiments of the above method, the subject is afflicted with the chronic or acute lung inflammatory disease is chronic obstructive pulmonary disease (COPD), pneumonia, asthma, acute lung injury, lung cancer or pulmonary impairment.

In some embodiments of the above method, the subject is afflicted with the chronic or acute lung inflammatory disease is COPD exacerbation induced by exposure to an environmental factor.

In some embodiments of the above method, the environmental factor is a particulate matter 2.5 microns or smaller.

In some embodiments of the above method, the chronic or acute lung inflammatory disease is chronic bronchitis or emphysema.

In some embodiments of the above method, the chronic or acute lung inflammatory disease is chronic bronchitis or emphysema.

In some embodiments of the above method, the chronic or acute lung inflammatory disease is bacterial pneumonia.

In some embodiments of the above method, the subject is normoglycemic.

In some embodiments of the above method, the subject is hyperglycemic.

In some embodiments, the method wherein the in the compound, R₁ is other than H.

In some embodiments, the method wherein the compound has the structure:

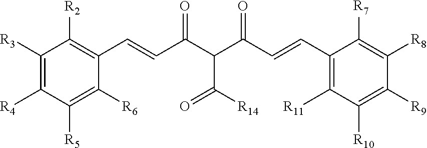

wherein R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

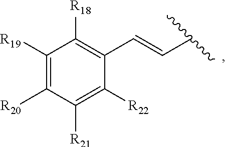

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl; R₁₈, R₁₉, R₂₁, and R₂₂ are each independently H, halogen, —NO₂, —CN, —NR₂₃R₂₄, —SR₂₃, —SO₂R₂₃, —COR₂₃, —OR₂₅, CF₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₉R₂₉, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, or C₂₋₁₀ alkynyl; and
wherein each occurrence of alkyl, alkenyl, or alkynyl is branched or unbranched, unsubstituted or substituted; and
or a salt thereof.

In some embodiments, the method wherein the compound has the structure:

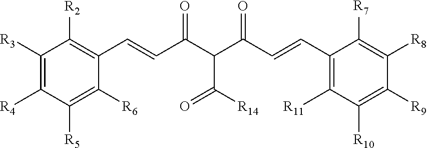

wherein R₁₄ is C₂₋₁₀ alkyl, C₂₋₁₀ alkenyl, C₂₋₁₀ alkynyl, heteroaryl, heterocyclyl, —OR₁₅, —NR₁₆R₁₇, or

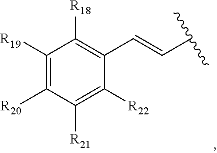

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, or C₂₋₁₀ alkynyl; and
wherein each occurrence of alkyl, alkenyl, or alkynyl is branched or unbranched, unsubstituted or substituted; and
or a salt thereof.

In some embodiments, the method wherein the compound has the structure:

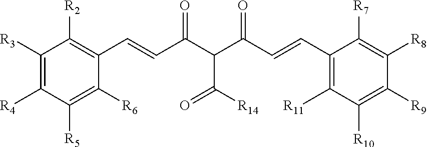

wherein R₁₄ is C₂₋₁₀ alkyl, C₂₋₁₀ alkenyl, C₂₋₁₀ alkynyl, heteroaryl, heterocyclyl, —OR₁₅, —NR₁₆R₁₇, or

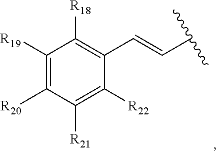

wherein R₁₅ is H, C₄₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₂₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
   wherein R₂₈ and R₂₉ are each, H, CF₃, C₁₋₁₀ alkyl, C₂₋₁₀ alkenyl, or C₂₋₁₀ alkynyl; and
wherein each occurrence of alkyl, alkenyl, or alkynyl is branched or unbranched, unsubstituted or substituted; and
or a salt thereof.

In some embodiments, the method wherein at least one of R₂, R₃, R₄, R₅, and R₆ and at least one of R₇, R₈, R₉, R₁₀, and R₁₁, are each, independently, —OR₂₈.

In some embodiments, the method wherein
R₁₄ is methoxy, —OR₁₅ or —NR₁₆R₁₇;
R₁₅ is H, C₃₋₁₀ alkyl, C₂₋₁₀ alkenyl, or C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
or a salt thereof.

In some embodiments, the method wherein
R₁₄ is methoxy or —NR₁₆R₁₇;
R₁₆ and R₁₇ are each, independently, H, C₁₋₁₀ alkyl, C₂₋₁₀ alkenyl, C₂₋₁₀ alkynyl, aryl, heteroaryl, or heterocyclyl; or a salt thereof.

In some embodiments, the method wherein
R₁₄ is —OR₁₅,
R₁₅ is H, C₃₋₁₀ alkyl, C₂₋₁₀ alkenyl, or C₂₋₁₀ alkynyl;
   or a salt thereof.

In some embodiments, the method wherein
R₁₄ is —NR₁₆R₁₇,
   wherein R₁₆ and R₁₇ are each, independently, H or aryl;
R₂, R₃, R₄, R₅, R₆, R₇, R₈, R₉, R₁₀, and R₁₁ are each independently, H, —NR₂₈R₂₉⁺, or —OR₂₈,
   wherein R₂₈ and R₂₉ are each, H or C₁₋₁₀ alkyl;
      or a salt thereof.

In some embodiments, the method wherein
R₁₄ is —NH-phenyl;
R₂, R₅, R₆, R₇, R₁₀, and R₁₁ are each H;
R₃, R₄, R₈, and R₉ are each, independently, H, —OH, or —OCH₃;
   or a salt thereof.

In some embodiments, the method wherein the compound has the structure

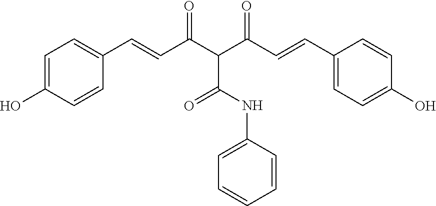 [SMILES: O=C(/C=C/c1ccc(O)cc1)C(C(=O)/C=C/c1ccc(O)cc1)C(=O)Nc1ccccc1]

or a pharmaceutically acceptable salt thereof.

In some embodiments, the one or more lipoxins are increased by 10% or more in the subject relative to a subject with normal levels of the one or more lipoxins which subject with normal levels does not have a disease associated with decreased levels of one or more lipoxins.

In some embodiments, the one or more lipoxins are increased by 20% or more in the subject relative to a subject with normal levels of the one or more lipoxins which subject with normal levels does not have a disease associated with decreased levels of one or more lipoxins.

In some embodiments, the one or more lipoxins are increased by 30% or more in the subject relative to a subject with normal levels of the one or more lipoxins which subject with normal levels does not have a disease associated with decreased levels of one or more lipoxins.

In some embodiments, the one or more lipoxins are increased by 40% or more in the subject relative to a subject with normal levels of the one or more lipoxins which subject with normal levels does not have a disease associated with decreased levels of one or more lipoxins.

In some embodiments, the one or more lipoxins are increased by 50% or more in the subject relative to a subject with normal levels of the one or more lipoxins which subject with normal levels does not have a disease associated with decreased levels of one or more lipoxins.

In some embodiments, the one or more lipoxins are increased by 10% or more in the subject. In some embodiments, the one or more lipoxins are increased by 20% or more in the subject. In some embodiments, the one or more lipoxins are increased by 30% or more in the subject. In some embodiments, the one or more lipoxins are increased by 40% or more in the subject. In some embodiments, the one or more lipoxins are increased by 50% or more in the subject. In some embodiments, the one or more lipoxins are increased by 100% or more in the subject. In some embodiments, the one or more lipoxins are increased by 200% or more in the subject.

In some embodiments, the one or more resolvins are increased by 10% or more in the subject. In some embodiments, the one or more resolvins are increased by 20% or more in the subject. In some embodiments, the one or more resolvins are increased by 30% or more in the subject. In some embodiments, the one or more resolvins are increased by 40% or more in the subject. In some embodiments, the one or more resolvins are increased by 50% or more in the subject. In some embodiments, the one or more resolvins are increased by 100% or more in the subject. In some embodiments, the one or more resolvins are increased by 200% or more in the subject.

In some embodiments, the one or more protectins are increased by 10% or more in the subject. In some embodiments, the one or more protectins are increased by 20% or more in the subject. In some embodiments, the one or more protectins are increased by 30% or more in the subject. In some embodiments, the one or more protectins are increased by 40% or more in the subject. In some embodiments, the one or more protectins are increased by 50% or more in the subject. In some embodiments, the one or more protectins are increased by 100% or more in the subject. In some embodiments, the one or more protectins are increased by 200% or more in the subject.

In some embodiments, the one or more maresins are increased by 10% or more in the subject. In some embodiments, the one or more maresins are increased by 20% or more in the subject. In some embodiments, the one or more maresins are increased by 30% or more in the subject. In some embodiments, the one or more maresins. are increased by 40% or more in the subject. In some embodiments, the one or more maresins are increased by 50% or more in the subject. In some embodiments, the one or more maresins are increased by 100% or more in the subject. In some embodiments, the one or more maresins are increased by 200% or more in the subject.

In some embodiments, the one or more anti-inflammatory cytokines are increased by 10% or more in the subject. In some embodiments, the one or more anti-inflammatory cytokines are increased by 20% or more in the subject. In some embodiments, the one or more anti-inflammatory cytokines are increased by 30% or more in the subject. In some embodiments, the one or more anti-inflammatory cytokines are increased by 40% or more in the subject. In some embodiments, the one or more anti-inflammatory cytokines are increased by 50% or more in the subject. In some embodiments, the one or more anti-inflammatory cytokines are increased by 100% or more in the subject. In some embodiments, the one or more anti-inflammatory cytokines are increased by 200% or more in the subject.

In some embodiments, the levels of one or more lipoxins is increased in the lungs of the subject.

In some embodiments, the subject in need thereof has decreased levels of one or more lipoxins due to a disease associated with decreased levels of one or more lipoxins.

An additional aspect of the invention provides analogs of the compound CMC2.24 that behave analogously to CMC2.24 in increasing lipoxin production and otherwise. Additional compounds (below) have been manufactured as described in PCT International Application WO 2010/132815 A9, the contents of which are hereby incorporated by reference. The analogs of CMC2.24 shown below have analogous activity to CMC2.24.

In some embodiments, the compound has the structure:

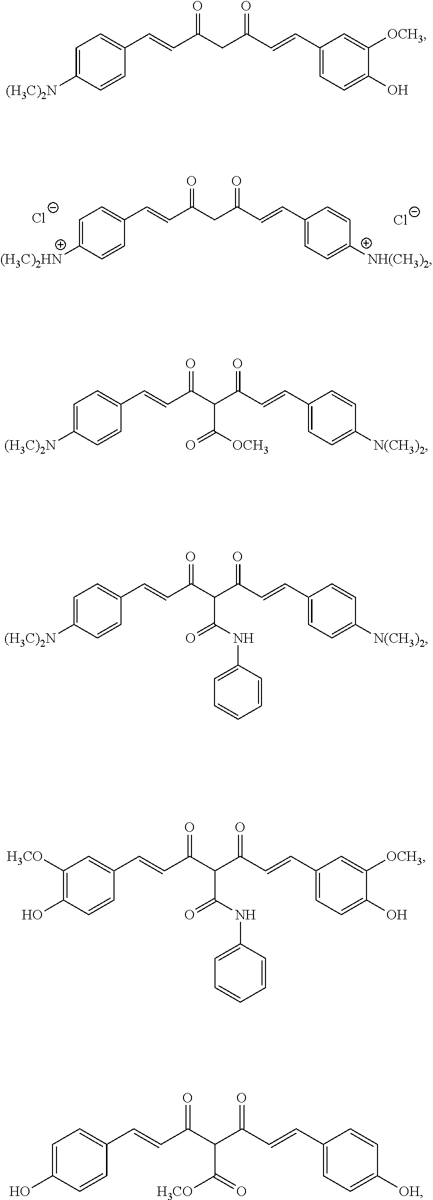

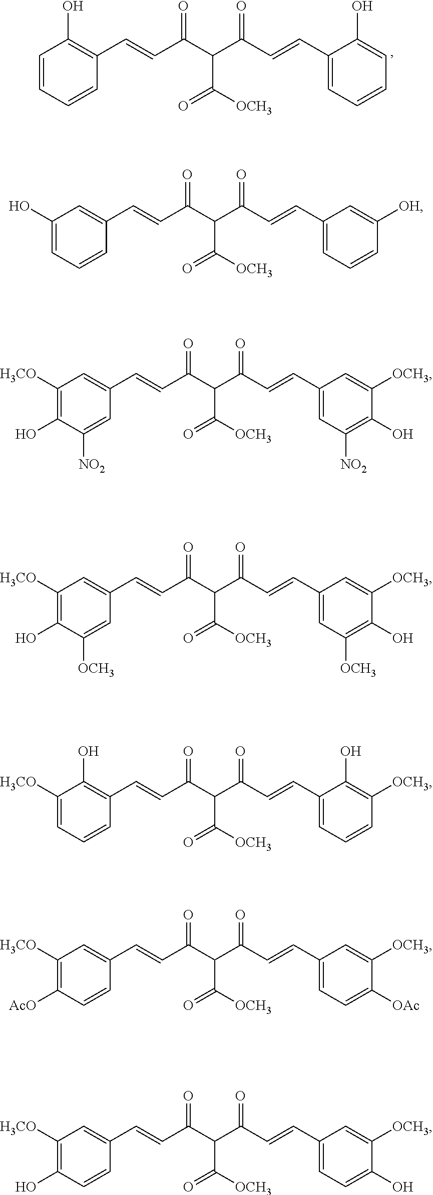

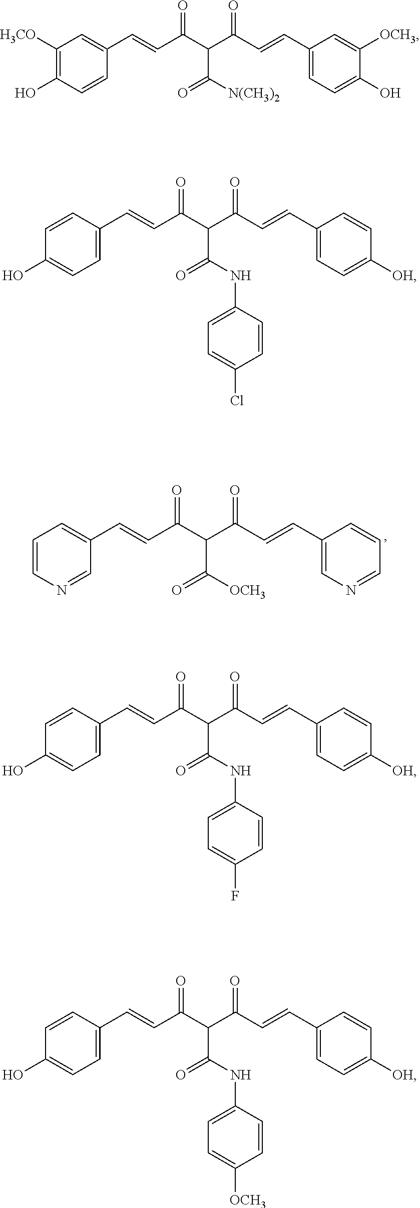 [SMILES: COc1cc(/C=C/C(=O)C(C(=O)/C=C/c2ccc(O)c(OC)c2)C(=O)N(C)C)ccc1O ; O=C(/C=C/c1ccc(O)cc1)C(C(=O)/C=C/c1ccc(O)cc1)C(=O)Nc1ccc(Cl)cc1 ; COC(=O)C(C(=O)/C=C/c1cccnc1)C(=O)/C=C/c1cccnc1 ; O=C(/C=C/c1ccc(O)cc1)C(C(=O)/C=C/c1ccc(O)cc1)C(=O)Nc1ccc(F)cc1 ; COc1ccc(NC(=O)C(C(=O)/C=C/c2ccc(O)cc2)C(=O)/C=C/c2ccc(O)cc2)cc1]

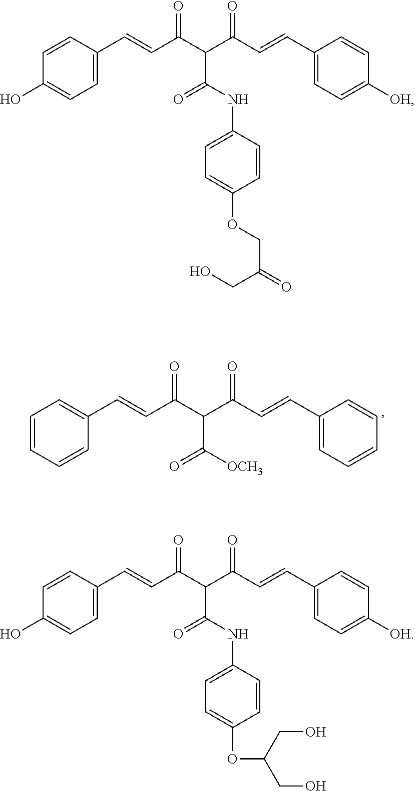 [SMILES: O=C(CO)COc1ccc(NC(=O)C(C(=O)/C=C/c2ccc(O)cc2)C(=O)/C=C/c2ccc(O)cc2)cc1 ; COC(=O)C(C(=O)/C=C/c1ccccc1)C(=O)/C=C/c1ccccc1 ; O=C(/C=C/c1ccc(O)cc1)C(C(=O)/C=C/c1ccc(O)cc1)C(=O)Nc1ccc(OC(CO)CO)cc1]

In one embodiment, a method of treating a disease or condition associated with decreased levels of one or more lipoxins in a subject afflicted therewith which comprises the following:
(a) determining the levels of the one or more lipoxins in cells isolated from the subject;
(b) comparing the levels of the one or more lipoxins in the cells relative to a predetermined reference level; and
(c) administering an effective amount of a compound having the structure:

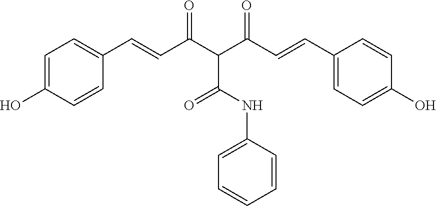 [SMILES: O=C(/C=C/c1ccc(O)cc1)C(C(=O)/C=C/c1ccc(O)cc1)C(=O)Nc1ccccc1]

to the subject if there are decreased levels of the one or more lipoxins in the cells as compared with the predetermined reference level.

In one embodiment, a method of treating a disease or condition associated with decreased levels lipoxin A4 in a subject afflicted therewith which comprises the following:
(a) determining the levels of lipoxin A4 in cells isolated from the subject;
(b) comparing the levels of lipoxin. A4 in the cells relative to a predetermined reference level; and
(c) administering an effective amount of a compound having the structure:

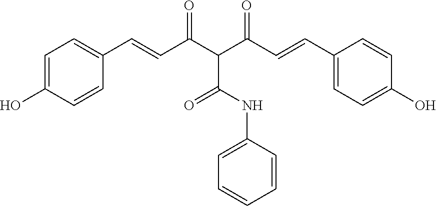 [SMILES: O=C(/C=C/c1ccc(O)cc1)C(C(=O)/C=C/c1ccc(O)cc1)C(=O)Nc1ccccc1]

to the subject if there are decreased levels of lipoxin A4 in the cells as compared with the predetermined reference level.

The compounds of the present invention increase production of 15-epi-LXA4 and lipoxin B4 in a similar manner to the increase of lipoxin A1.

The compounds of the present invention increase production of resolvins, protectins and maresins in a similar manner to the increase of lipoxin A1.

The present invention provides a method of increasing production of one or more resolvins in a normoglycemic subject in need thereof comprising administering to the subject an amount of a compound of the present invention so as to thereby increase production of the one or more resolvins in the subject.

The present invention provides a method of increasing production of one or more protectins in a normoglycemic subject in need thereof comprising administering to the subject an amount of a compound of the present invention so as to thereby increase production of the one or more protectins in the subject.

The present invention provides a method of increasing production of one or more maresins in a normoglycemic subject in need thereof comprising administering to the subject an amount of a compound of the present invention so as to thereby increase production of the one or more maresins in the subject.

The present invention also provides a method of treating a subject afflicted with a disease associated with decreased levels of one or more resolvins comprising inducing production of the one or more resolvins in the subject by administering to the subject an amount of a compound of the present invention so as to thereby treat the subject afflicted with the disease.

The present invention also provides a method of treating a subject afflicted with a disease associated with decreased levels of one or more protectins comprising inducing production of the one or more protectins in the subject by administering to the subject an amount of a compound of the present invention so as to thereby treat the subject afflicted with the disease.

The present invention also provides a method of treating a subject afflicted with a disease associated with decreased levels of one or more maresins comprising inducing production of the one or more maresins in the subject by administering to the subject an amount of a compound of the present invention so as to thereby treat the subject afflicted with the disease.

In some embodiments, the subject is afflicted with pneumonia.

In some embodiments, the disease associated with decreased levels of one or more lipoxins is pneumonia.

In some embodiments, the subject is afflicted with pulmonary bacterial pneumonia.

In some embodiments, the disease associated with decreased levels of one or more lipoxins is pulmonary bacterial pneumonia.

In some embodiments, the pulmonary bacterial pneumonia is caused by Staphylococcus aureus.

Use of any compound disclosed in the present application or a pharmaceutically acceptable salt or ester thereof, for increasing production of one or more lipoxins in a subject.

Use of any compound disclosed in the present application or a pharmaceutically acceptable salt or ester thereof, for increasing production of one or more resolvins in a subject.

Use of any compound disclosed in the present application or a pharmaceutically acceptable salt or ester thereof, for increasing production of one or more protectins in a subject.

Use of any compound disclosed in the present application or a pharmaceutically acceptable salt or ester thereof, for increasing production of one or more maresins in a subject.

Use of any compound disclosed in the present application or a pharmaceutically acceptable salt or ester thereof, for the manufacture of a medicament for use in treating a disease associated with decreased levels of one or more lipoxins.

Use of any compound disclosed in the present application or a pharmaceutically acceptable salt or ester thereof, for the manufacture of a medicament for use in treating a disease associated with decreased levels of one or more resolvins.

Use of any compound disclosed in the present application or a pharmaceutically acceptable salt or ester thereof, for the manufacture of a medicament for use in treating a disease associated with decreased levels of one or more protectins.

Use of any compound disclosed in the present application or a pharmaceutically acceptable salt or ester thereof, for the manufacture of a medicament for use in treating a disease associated with decreased levels of one or more maresins.

Any compound disclosed in the present application or a pharmaceutically acceptable salt or ester thereof for use in treating a disease associated with decreased levels of one or more lipoxins.

Any compound disclosed in the present application or a pharmaceutically acceptable salt or ester thereof for use in treating a disease associated with decreased levels of one or more reslovins.

Any compound disclosed in the present application or a pharmaceutically acceptable salt or ester thereof for use in treating a disease associated with decreased levels of one or more protectins.

Any compound disclosed in the present application or a pharmaceutically acceptable salt or ester thereof for use in treating a disease associated with decreased levels of one or more maresins.

The present invention provides a pharmaceutical composition comprising a compound disclosed in the present application for use in treating a disease associated with decreased levels of one or more lipoxins, a disease associated with decreased levels of one or more reslovins, a disease associated with decreased levels of one or more protectins or a disease associated with decreased levels of one or more maresins.

The present invention provides a pharmaceutical composition comprising a compound disclosed in the present application for use in increasing production of one or more lipoxinsin a subject, for use in increasing production of one or more resolvins in a subject, for use in increasing production of one or more protectins in a subject, or for use in increasing production of one or more maresins in a subject.

Use of any compound disclosed in the present application or a pharmaceutically acceptable salt or ester thereof, for the manufacture of a medicament for use in treating any of the diseases of conditions disclosed herein.

Any compound disclosed in the present application or a pharmaceutically acceptable salt or ester thereof for use in treating any of the diseases or conditions disclosed herein.

The present invention provides a pharmaceutical composition comprising a compound disclosed in the present application for use in treating any of the diseases or conditions disclosed herein.

In some embodiments of any of the disclosed methods, the compound is administered to the subject in an amount between about 0.5 mg/kg and about 10.0 mg/kg body weight of the subject/day.

In some embodiments of any of the disclosed methods, the compound is administered to the subject in an amount between about 1 mg/kg and about 10.0 mg/kg body weight of the subject/day.

In some embodiments of any of the disclosed methods, the compound is administered to the subject in an amount between about 0.5 mg/kg and about 7.5 mg/kg body weight of the subject/day.

In some embodiments of any of the disclosed methods, the compound is administered to the subject in an amount between about 1 mg/kg and about 5 mg/kg body weight of the subject/day.

In some embodiments of any of the disclosed methods, the compound is administered to the subject in an amount between about 2 mg/kg and about 5 mg/kg body weight of the subject/day.

In some embodiments of any of the disclosed methods, the compound is administered to the subject in an amount between about 2 mg/kg and about 4 mg/kg body weight of the subject/day.

In some embodiments of any of the disclosed methods, the compound is administered to the subject in an amount between about 2.5 mg/kg and about 4.5 mg/kg body weight of the subject/day.

In some embodiments of any of the disclosed methods, the compound is administered to the subject in an amount between about 0.5 mg/kg and about 10 mg/kg body weight of the subject/day.

In some embodiments of any of the disclosed methods, the compound is administered to the subject in an amount between about 1 mg/kg and about 50 mg/kg body weight of the subject/day.

In some embodiments of any of the disclosed methods, the compound is administered to the subject in an amount between about 10 mg/kg and about 10 mg/kg body weight of the subject/day.

In some embodiments of any of the disclosed methods, the compound is administered to the subject in an amount of about 1 mg/kg body weight of the subject/day, 3 mg/kg body weight of the subject/day, 5 mg/kg body weight of the subject/day, 10 mg/kg body weight of the subject/day, 30 mg/kg body weight of the subject/day, 40 mg/kg body weight of the subject/day or 50 mg/kg body weight of the subject/day.

In some embodiments of any of the disclosed methods, the compound is administered daily to the subject.

The method of the present invention increases production of lipoxins and reduces production of proinflammatory cytokines in the subject, thereby creating a non-inflammatory balance.

The method of the present invention increases amounts of lipoxins and reduces amounts proinflammatory cytokines in the subject, thereby creating a non-inflammatory balance.

As used herein, “disease associated with decreased levels of one or more lipoxins” is any disease other than diabetes wherein the subject has decreased levels of one or more lipoxins. The levels of lipoxin A4 have been reported to be decreased in chronic airway inflammatory disease such as asthma, chronic obstructive pulmonary disease and cystic fibrosis (Bonnans, C. et al. 2002; Karp, C L et al. 2004; Planaguma, A. et al. 2008; Balode, L. et al. 2012).

As used herein, “disease associated with decreased levels of one or more lipoxins” does not encompass a skin wound which is any injury in which the skin of a subject is torn, pierced, cut, or otherwise broken, and any disruption of the skin which results from an injury, an infection, from direct contact with an allergen or irritant, or from an autoimmune disease. Examples of skin wounds include but are not limited to cuts, abrasions, punctures, blisters, boils, wheals, burns, rashes, contact dermatitis, bites and psoriasis.

As used herein, “disease associated with decreased levels of one or more lipoxins” does not encompass a wound which is any injury in which an external surface, internal mucosa, oral lining or any epithelial tissue of a subject is torn, pierced, cut, abraded or otherwise broken, and any disruption of an external surface, internal mucosa, oral lining or any epithelial tissue of a subject which results from an injury, an infection, from direct contact with an allergen or irritant, or from an autoimmune disease. A non-limiting example of an autoimmune disease is pemphigoid.

As used herein, “disease associated with decreased levels of one or more resolvins”, “disease associated with decreased levels of one or more protectins” and “disease associated with decreased levels of one or more maresins” is any disease other than diabetes wherein the subject has decreased levels of one or more resolvins, protectins or maresins.

As used herein, “disease associated with decreased levels of one or more resolvins”, “disease associated with decreased levels of one or more protectins” and “disease associated with decreased levels of one or more maresins” do not encompass a skin wound which is any injury in which the skin of a subject is torn, pierced, cut, or otherwise broken, and any disruption of the skin which results from an injury, an infection, from direct contact with an allergen or irritant, or from an autoimmune disease. Examples of skin wounds include but are not limited to cuts, abrasions, punctures, blisters, boils, wheals, burns, rashes, contact dermatitis, bites and psoriasis.

As used herein, “disease associated with decreased levels of one or more resolvins”, “disease associated with decreased levels of one or more protectins” and “disease associated with decreased levels of one or more maresins” do not encompass a wound which is any injury in which an external surface, internal mucosa, oral lining or any epithelial tissue of a subject is torn, pierced, cut, abraded or otherwise broken, and any disruption of an external surface, internal mucosa, oral lining or any epithelial tissue of a subject which results from an injury, an infection, from direct contact with an allergen or irritant, or from an autoimmune disease. A non-limiting example of an autoimmune disease is pemphigoid.

In some embodiments, the disease associated with decreased levels of one or more lipoxins is rheumatoid arthritis, osteoarthritis, psoriatic arthritis, periodontitis, inflammatory bowel disease, irritable bowel syndrome, psoriasis, ankylosing spondylitis, Sjogren's syndrome, multiple sclerosis, ulcerative colitis, Crohn's disease, systemic lupus erythematosus, lupus nephritis, psoriasis, celiac disease, vasculitis, atherosclerosis, cystic fibrosis, asthma, or chronic obstructive pulmonary disease (COPD).

There is a vast array of diseases exhibiting an inflammatory component. These include but are not limited to: inflammatory joint diseases (e.g., rheumatoid arthritis, osteoarthritis, polyarthritis and gout), chronic inflammatory connective tissue diseases (e.g., lupus erythematosus, scleroderma, Sjorgen's syndrome, poly- and dermatomyositis, vasculitis, mixed connective tissue disease (MCTD), tendonitis, synovitis, bacterial endocarditis, osteomyelitis and psoriasis), chronic inflammatory lung diseases (e.g., chronic respiratory disease, pneumonia, fibrosing alveolitis, chronic bronchitis, chronic obstructive pulmonary disease (COPD), bronchiectasis, emphysema, silicosis and other pneumoconiosis and tuberculosis), chronic inflammatory bowel and gastro-intestinal tract inflammatory diseases (e.g., ulcerative colitis and Crohn's disease), chronic neural inflammatory diseases (e.g., chronic inflammatory demyelinating polyradiculoneuropathy, chronic inflammatory demyelinating polyneuropathy, multiple sclerosis, Guillan-Barre Syndrome and myasthemia gravis), other inflammatory diseases (e.g., mastitis, laminitis, laryngitis, chronic cholecystitis, Hashimoto's thyroiditis, inflammatory breast disease); chronic inflammation caused by an implanted foreign body in a wound; and acute inflammatory tissue damage due to muscle damage after eccentric exercise (e.g., delayed onset muscle soreness—DOMS).

The usual mode of treatment for chronic inflammatory conditions is by administration of non-steroidal anti-inflammatory drugs (NSAID's) such as Diclofenac, Ibuprofen, Aspirin, Phenylbutazone, indomethacin, Naproxen and Piroxicam. Although NSAID's can be effective, they are known to be associated with a number of side effects and adverse reactions

Any of the diseases disclosed herein associated with decreased levels of one or more lipoxins may also be a “disease associated with decreased levels of one or more resolvins”, a “disease associated with decreased levels of one or more protectins” or a “disease associated with decreased levels of one or more maresins”.

Various pro-resolving lipid mediators increased by the method of the present invention are described in Buckley C. D, Gilroy D. W, Serhan C. N. Proresolving Lipid Mediators and Mechanisms in the Resolution of Acute Inflammation. Immunity 2014, 40 (3), 315-27, the contents of which are hereby incorporated by reference.

The CMC's disclosed herein have improved solubility and greater zinc binding capability and enhanced therapeutic anti-inflammatory effects and efficacy in vivo relative to curcumin.

CMC2.24 has improved solubility and greater zinc binding capability and enhanced therapeutic anti-inflammatory effects and efficacy in vivo relative to CMC2.5.

In an embodiment, the method wherein the subject is other than a diabetic subject. In an embodiment, the method wherein the subject is other than a subject diagnosed with diabetes.

In an embodiment, the method wherein the subject is other than a hypo- or hyperglycemic subject.

In some embodiments, the compound is solubilized in a non-toxic organic solubilizing agent. A non-limiting example of a non-toxic organic solubilizing agent is N-methylglucamine, which is also known as “meglumine”.

This invention provides a pharmaceutical composition comprising a pharmaceutically acceptable carrier and of the above compounds.

The compounds of the present invention increase production of lipoxins, resolvins and/or anti-inflammatory cytokines in a subject. Molecules such as cytokines, resolvins and lipoxins may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention. In particular, reduced levels of lipoxins, resolvins and/or anti-inflammatory cytokines are associated with various disease including, but not limited to, inflammatory diseases.

Variations on the following general synthetic methods (Pabon, H. 1964) will be readily apparent to those skilled in the art and are used to prepare the compounds of the method of the present invent ion.

Scheme 1. Synthesis of curcumin analogues.

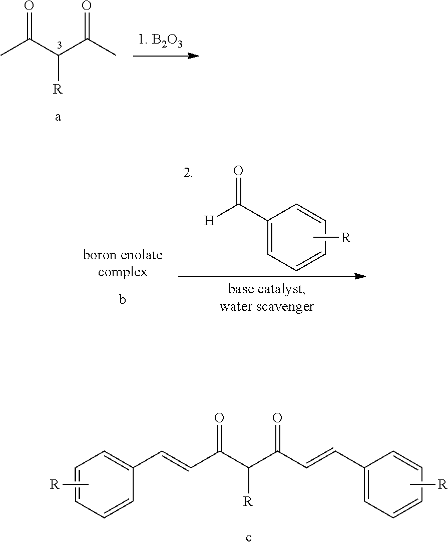

The synthesis of the curcumin analogues of the present invention can be carried out according to general Scheme 1. The R groups designate any number of generic substituents.

The starting material is provided by 2,4-pentanedione, which is substituted at the 3-carbon (see compound a). The desired substituted 2,4-pentanedione may be purchased from commercial sources or it may be synthesized using conventional functional group transformations well-known in the chemical arts, for example, those set forth in Organic Synthesis, Michael B. Smith, (McGraw-Hill) Second ed. (2001) and March's Advanced Organic Chemistry: Reactions, Mechanisms, and Structure, Michael B. Smith and Jerry March, (Wiley) Sixth ed. (2007), and specifically by Bingham and Tyman (45) and in the case of 3-aryl-aminocarbonyl compounds by Dieckman, Hoppe and Stein (46), the contents of which are hereby incorporated by reference. 2,4-pentanedione a is reacted with boron trioxide to form boron enolate complex b.

Boron enolate complex b is a complex formed by coordination of the enolate of compound a with boron. It is understood by those having ordinary skill in the art that the number of compound a enolates that may coordinate to boron as well as the coordination mode, i.e. monodentate versus bidentate, are variable so long as reaction, such as Knoevenagel condensation, at the C-3 carbon of the 2,4-pentanedione is suppressed.

Boron enolate complex b is then exposed to a benzaldehyde compound in the presence of a base catalyst and a water scavenger to form curcumin analogue c via aldol condensation. The ordinarily skilled artisan will appreciate that the benzaldehyde may possess various substituents on the phenyl ring so long as reactivity at the aldehyde position is not hindered. Substituted benzaldehyde compounds may be purchased from commercial sources or readily synthesized using aryl substitution chemistry that is well-known in the art. Suitable base catalysts for the aldol step include, but are not limited to, secondary amines, such as n-butylamine and n-butylamine acetate, and tertiary amines.

Suitable water scavengers include, but are not limited to, alkyl borates, such as trimethyl borate, alkyl phosphates, and mixtures thereof. Other suitable reaction parameters have also been described by Krackov and Bellis in U.S. Pat. No. 5,679,864, the content of which is hereby incorporated by reference.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹³C, ¹³C. or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n=1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of 0, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

As used herein, the term “electron-withdrawing group” refers to a substituent or functional group that has the property of increasing electron density around itself relative to groups in its proximity. Electron withdrawing property is a combination of induction and resonance. Electron withdrawal by induction refers to electron cloud displacement towards the more electronegative of two atoms in a σ-bond. Therefore, the electron cloud between two atoms of differing electronegativity is not uniform and a permanent state of bond polarization occurs such that the more electronegative atom has a slight negative charge and the other atom has a slight positive charge. Electron withdrawal by resonance refers to the ability of substituents or functional groups to withdraw electron density on the basis of relevant resonance structures arising from p-orbital overlap. Suitable electron-withdrawing groups include, but are not limited to, —CN, —CF₃, halogen, —NO₂, —OCF₃, —OR₁₂, —NHCOR₁₂, —SR₁₂, —SO₂R₁₃, —COR₁₄, —CSR₁₄, —CNR₁₄, —C(═NR₁₂)R₁₄, —C(═NH)R₁₄, —SOR₁₂—, —POR₁₂, —P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —ORᵢₛ, —NR₁₆R₁₇, or

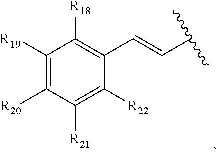

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₁₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, C(═NR₂₄)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl.

While curcumin has been known to bind metal ions such as those of copper, iron, and zinc, affinity for zinc has been shown to be weak.

In the subject invention, the biological activity of curcumin analogues is attributed in part to their ability to access and bind zinc ions and an enhanced solubility. This invention describes that the enhancement of zinc binding affinity through the installation of electron-withdrawing and electron-donating groups at strategic locations, namely the C-4 carbon and the aryl rings, on the curcumin skeleton.

Without wishing to be bound by theory, it is believed that zinc binding affinity arises from increased stability of the curcumin enolate formed by removal of hydrogen from the C-4 carbon, which then proceeds to form a complex with zinc. The stability of a carbanion, including an enolate, is directly related to the acidity of the ionizable hydrogen, such as an enolic hydrogen. In general, the stability of an enolate increases with increasing acidity of the enolic hydrogen. Herein, the enolic hydrogen refers to the hydrogen atom connected to the C-4 carbon of the curcumin skeleton.

The acidity of the enolic hydrogen of curcumin and its analogues can be enhanced by incorporation of an electron-withdrawing group at the C-4 carbon. Substituents which delocalize negative charge will enhance acidity and stability of the resulting carbanion, such as an enolate. Again, without wishing to be bound by theory, it is believed that the electron-withdrawing group allows the negative charge of the enolate to be delocalized into the electron-withdrawing group, thereby stabilizing the enolate, enhancing its stability, and increasing its zinc binding affinity.

The electronic characteristics of the aryl rings of curcumin are also relevant for enhancing zinc binding affinity and biological activity. Electron-donating groups on the aryl portions of the curcumin skeleton improve its activity. The incorporation of such electron-donating groups on the aryl rings may affect one or more factors, including enhancement of water solubility and improvement of cation-pi interactions. Without wishing to be bound by theory, the installation of electron-donating groups on the aryl rings, in conjunction with the choice of C-4 electron-withdrawing group, is believed to increase electron polarization within the molecule such that intermolecular dipole-dipole forces with surrounding water molecules is enhanced, thereby increasing water solubility. Electron-donating groups may also increase water solubility by enhancing hydrogen-bonding interactions with surrounding water molecules. Furthermore, with respect to cation-pi interactions, it is believed that electron-donating groups increase electron density on the aryl rings, thereby enhancing the aryls' ability to recognize and/or bind to cations or cation-containing proteins.

The choice of electron-withdrawing groups on the C-4 carbon and the choice of electron-donating groups on the aryl rings may be chosen using techniques well known by the ordinarily skilled artisan. In general, the electron donating ability of common substituents suitable for use on the aryl rings can be estimated by their Hammett c values. The Hammett σₚₐᵣₐ value is a relative measurement comparing the electronic influence of the substituent in the para position of a phenyl ring to the electronic influence of a hydrogen substituted at the para position. Typically for aromatic substituents in general, a negative Hammett σₚₐᵣₐ value is indicative of a group or substituent having an electron-donating influence on a pi electron system (i.e., an electron-donating group) and a positive Hammett σₚₐᵣₐ value is indicative of a group or substituent having an electron-withdrawing influence on a pi electron system (i.e., an electron-withdrawing group).

Similarly, Hammett σₘₑₜₐ value is a relative measurement comparing the electronic influence of the substituent in the meta position of a phenyl ring to the electronic influence of a hydrogen substituted at the meta position. A list of Hammett σₚₐᵣₐ and σₘₑₜₐ values for common substituents can be found in Lowry and Richardson, “Mechanism and Theory in Organic Chemistry”, 3rd ed, p. 144. The effect of some substituents, including some electron-withdrawing groups, on C—H acidity can also be found on page 518 in Lowry and Richardson, “Mechanism and Theory in Organic Chemistry”, 3rd ed, the content of which is hereby incorporated by reference.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; November 15, 2(09) and “Approved Drug Products with Therapeutic Equivalence Evaluations” (U.S. Department Of Health And Human Services, 30th edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1965); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

As used herein, “treating” means preventing, slowing, halting, or reversing the progression of a disease or condition. Treating may also mean improving one or more symptoms of a disease or condition.

As used herein, “about” in the context of a numerical value or range means±10% of the numerical value or range recited or claimed, unless the context requires a more limited range.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

Chemically-modified curcumins may be relatively insoluble in water. Such compounds may be solubilized in a safe organic solubilizing agent, such as meglumine (ie., N-methyl glucamine which is a deoxy(methylamino) glucitol, a derivative of glucose) to solubilize such compounds to improve their efficacy systemically, e.g. by swallowing a teaspoon of a composition comprising a compound of the invention and meglumine qd or even by I.V. injection.

The compounds of the present invention can be synthesized according to methods described in PCT International Publication No. WO 2010/132815 A9. Variations on those general synthetic methods will be readily apparent to those of ordinary skill in the art and are deemed to be within the scope of the present invention.

The National Institutes of Health (NIH) provides a table of Equivalent Surface Area Dosage Conversion Factors below (Table A) which provides conversion factors that account for surface area to weight ratios between species.

TABLE A
Equivalent Surface Area Dosage Conversion Factors

| | To | | | | | |
|---|---|---|---|---|---|---|
| | | Mouse | Rat | Monkey | Dog | Man |
| | | 20 g | 150 g | 3 kg | 8 kg | 60 kg |
| From | Mouse | 1 | 1/2 | 1/4 | 1/6 | 1/12 |
| | Rat | 2 | 1 | 1/2 | 1/4 | 1/7 |
| | Monkey | 4 | 2 | 1 | 3/5 | 1/3 |
| | Dog | 6 | 4 | 1 2/3 | 1 | 1/2 |
| | Man | 12 | 7 | 3 | 2 | 1 |

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Example 1. CMC2.24 Normalizes IL-1β and IL-6 Levels

### Experimental Details

Adult rats were made diabetic by I.V. injection of streptozotocin; non-diabetic rats (NDC) served as controls. Half of the diabetics (blood glucose >500 mg/dl) were orally administered CMC2.24 (30 mg/kg) once per day for 3 weeks; untreated diabetics (UD) received vehicle alone. Thioglycollate- and glycogen-elicited PEs were collected at 4 days or 4 hours, respectively, to harvest macrophages and PMNs. The cells were counted and chemotactic activity assessed fluorometrically using a cell migration assay; matrix metalloproteinases (MMPs) in the cell-free exudates (CFEs) and in cell culture were analyzed by gelatin zymography, and cytokine levels were analyzed by ELISA.

Adult rats were induced to be type I diabetic by I.V. injection of streptozotocin (70 mg/kg). Non-diabetic rats served as controls. 30 mg/kg of CMC 2.24 was administered daily by oral gavage to STZ-diabetic rats for three weeks. The control diabetic rats received vehicle alone. Thioglycollate- and glycogen-elicited PEs were collected at 4 days or 4 hours prior to sacrifice, respectively, to harvest macrophages and PMNs. The cells were counted and chemotactic activity was analyzed by Boyden Chamber chemotaxis assay. MMP-2 and MMP-9 levels in the cell-free exudates (CFEs) and in cell culture were analyzed by gelatin zymography, and cytokine levels were analyzed by ELISA.

### Results

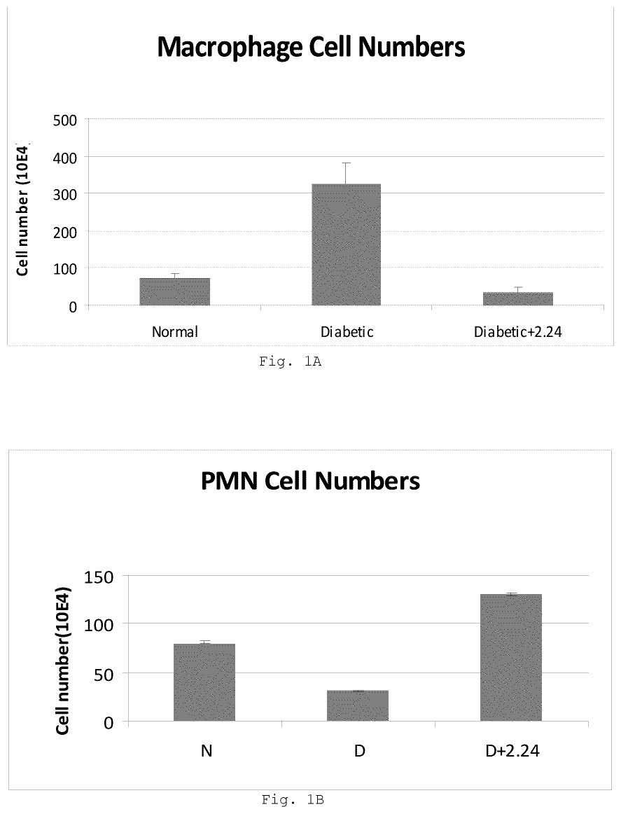

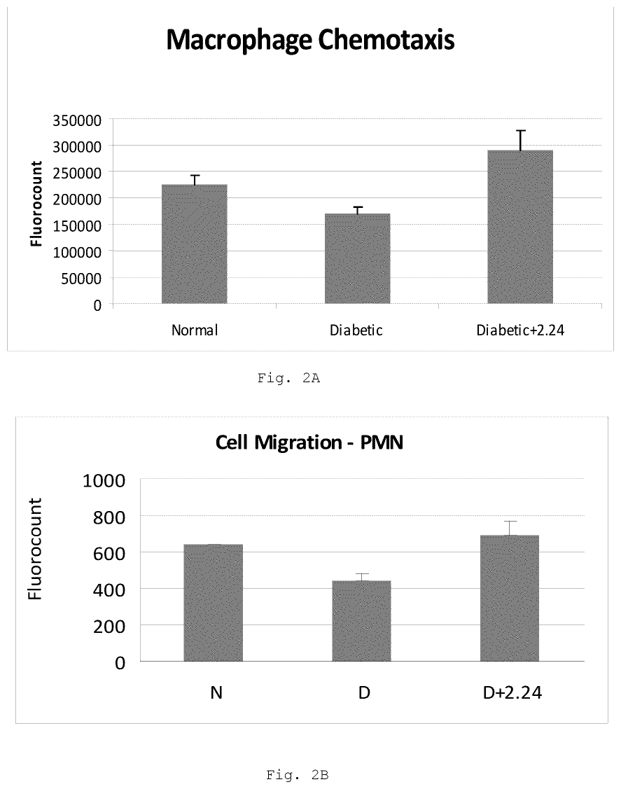

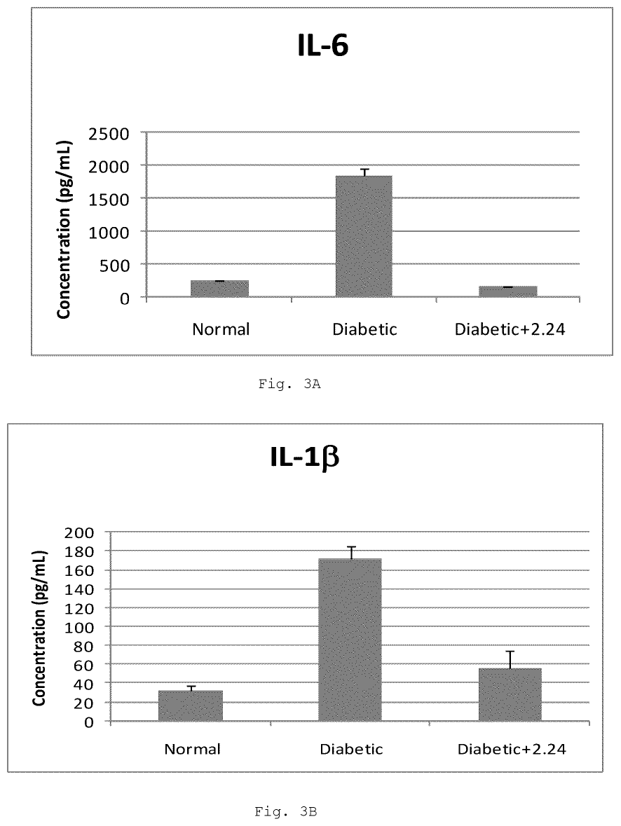

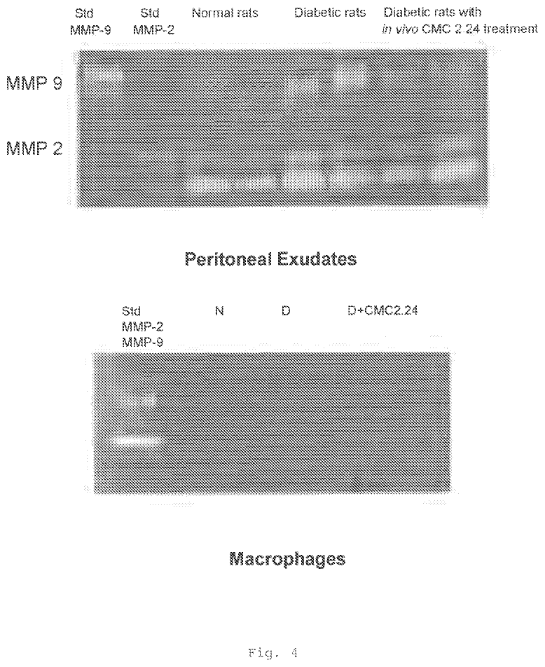

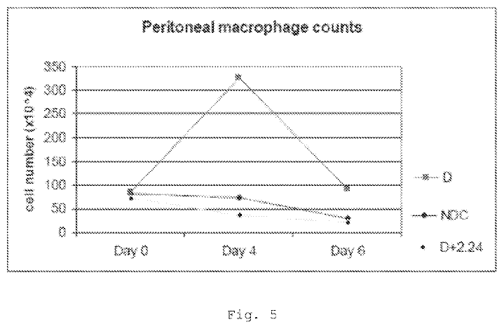

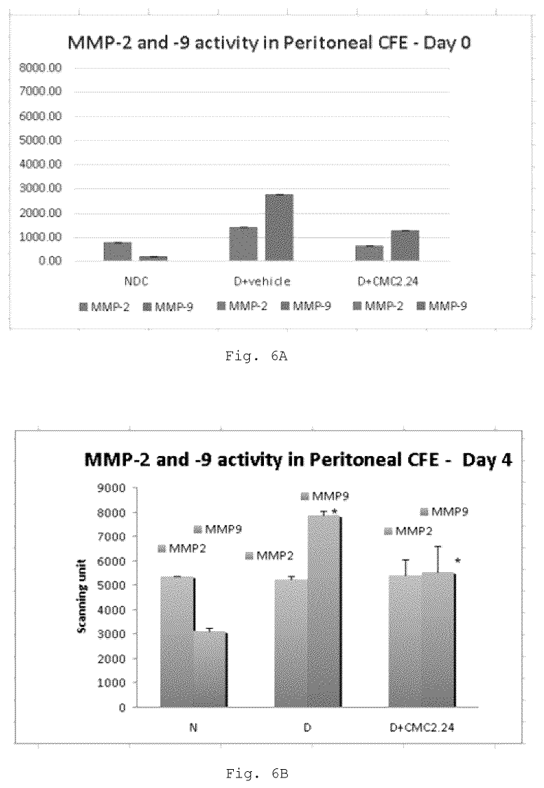

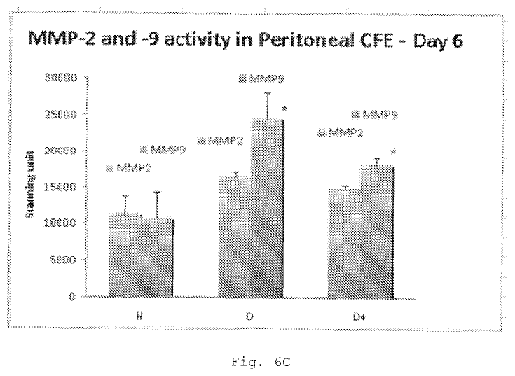

The polymorphonuclear leukocyte (PMNs) and macrophages from the UD rats (compared to the NDC rats) exhibited a significant (P<0.05) 31% and 24% reduction in chemotactic activity, respectively, as well as abnormal cell counts in the peritoneal exudates (PEs); all of these changes were “normalized” by CMC2.24 treatment (FIGS. 1 and 2). Macrophages from UD rats secreted 143% and 620% more IL-15 and IL-6, respectively, than the NDC rats, and both cytokines were reduced to normal levels by the CMC2.24 in vivo treatment (FIG. 3). Both the PE macrophages and the CFEs, from the UD rats, exhibited elevated MMP-9 levels, and CMC2.24 treatment reduced this 92 kDa gelatinase to normal levels (FIG. 4) (only low levels of mediators were seen in the PMN cultures).

Diabetes in rats modulates PMN and macrophage accumulation and activity in peritoneal exudates, and these abnormalities are “normalized” by oral administration of a pleiotropic MMP-inhibitor, CMC2.24, without affecting the severity of hyperglycemia in the diabetic rats.

### Example 2. CMC2.24 Normalizes IL-10 Levels

### Experimental Details

Adult rats were made diabetic by I.V. injection of streptozotocin; non-diabetic rats (N; n=6) served as controls. Half of the diabetics (blood glucose >500 mg/dl) were orally administered CMC2.24 (30 mg/kg) once per day for 3 weeks; untreated diabetics (D) and N rats received vehicle alone. PEs were collected at time-0 (resident macrophages), and at day 4 and day 6 after peritoneal thioglycollate injection. The PE macrophages were counted (hemocytometer); matrix metalloproteinases (MMPs) in the cell-free exudates (CFEs) were analyzed by densitometric analysis of gelatin zymograms, and IL-10 levels in cell culture, in CFE, and in serum were analyzed by ELISA.

### Results

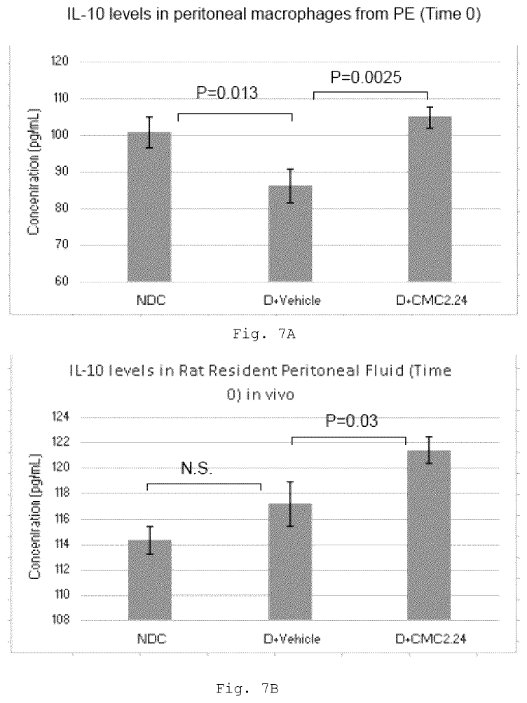

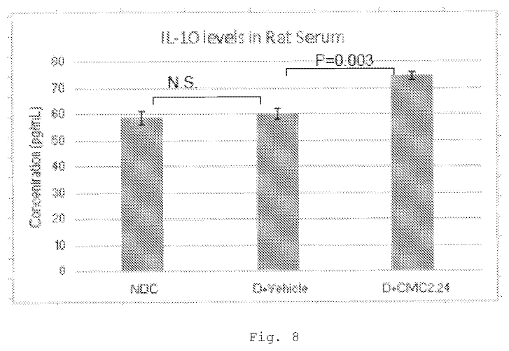

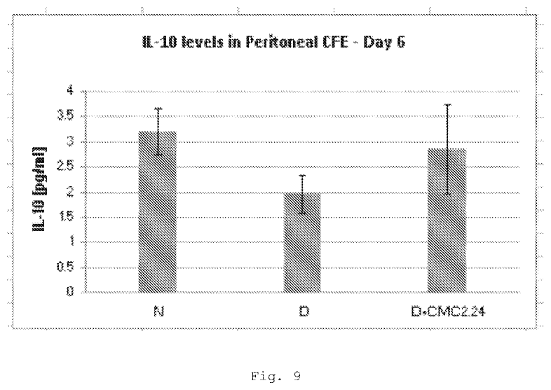

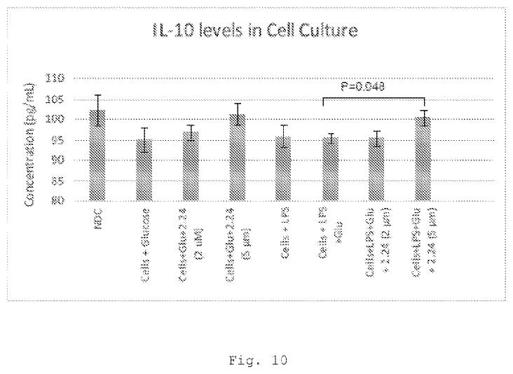

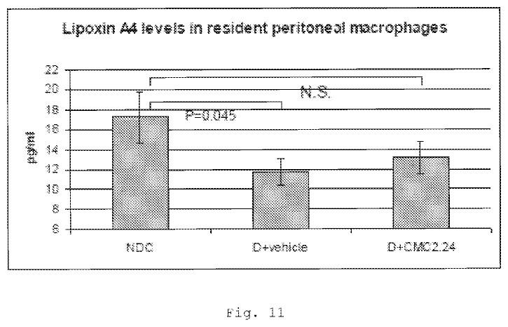

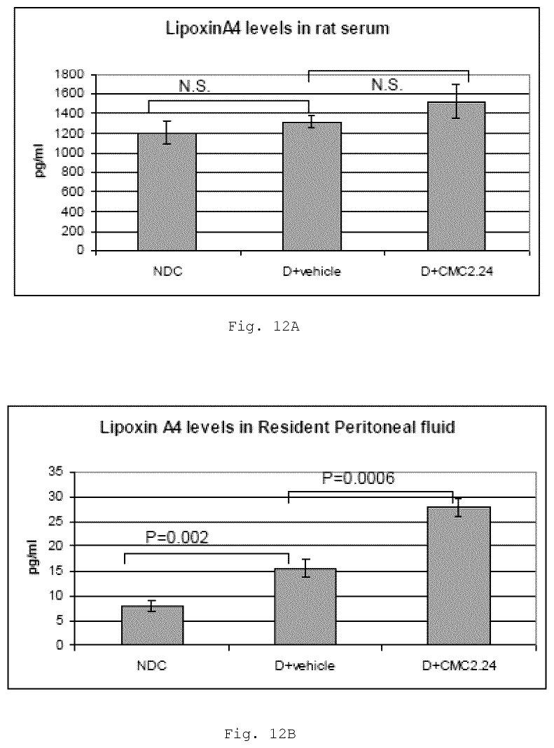

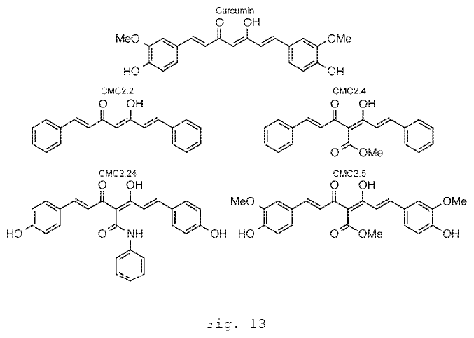

The PE macrophages at day 0, 4 and 6 days after thioglycollate injection in the D rats appeared to be increased compared to the N rats (FIG. 5). MMP-9 (including the homo- and hetero-dimer) levels in the CFEs of D rats were increased 960% (p<0.05) at time-0, compared to N rats; MMP-2 levels showed minimal changes (FIG. 6A). At day 4 and 6, again MMP-9 was significantly increased (p<0.05) in the D rats vs N rats, however CMC2.24 treatment of the diabetics “normalized” this MMP (FIG. 6B-C). Regarding cytokine analysis, proinflammatory IL-6 appeared increased, while pro-resolvin IL-10 was decreased, in the D rat PEs compared to N, IL-10 appeared to be “normalized” by CMC2.24 treatment (FIGS. 7-9).

IL-10 levels were also measured in cell culture. The effect of high glucose (550 mg/dL) & P. gingivalis LPS (endotoxin) on IL-10 secretion by macrophages from normal (NDC) rats was evaluated and compared to those treated with CMC2.24 (FIG. 10). IL-10 appeared to be “normalized” by CMC2.24 treatment at 2 μM (LPS) or 5 μM (both high glucose and LPS).

Untreated diabetic rats, when compared to non-diabetic controls, exhibited: (i) Abnormal macrophage counts in peritoneal exudates at Day 0, 4 and 6, and abnormal PMN counts at 4 hours; in addition, both types of inflammatory cells exhibited impaired chemotaxis; (ii) higher levels of MMP-9 in PE at Day 0, 4, and 6, (iii) decreased IL-10 levels (and increased pro-inflammatory cytokines, IL-15 and IL-6) in D peritoneal macrophages and CFE.

In vivo treatment of diabetic rats with CMC 2.24 showed: (i) “normalization” of numbers in macrophages in PE, (ii) reduction in MMP-9 and upregulating of IL-10 levels to near normal levels without affecting the severity of hyperglycemia in the diabetic rats.

### Example 3. CMC2.24 Increases Lipoxin A4 Levels

### Experimental Details

Adult rats were made diabetic by I.V. injection of streptozotocin; non-diabetic rats served as controls (n=6 rats per group; all groups). Half of the diabetics (blood glucose >500 mg/dl) were orally administered CMC2.24 (30 mg/kg) once per day for 3 weeks; untreated diabetics (D) and N rats received vehicle alone. PEs were collected at time-0 (ie, before thioglycollate injection into peritoneal cavity). Resident macrophages were isolated from PEs, then incubated in cell culture for 18 hours (370 C; 95% air/3% CO2 atmosphere). Lipoxin A4 levels were measured by ELISA (a) in cell culture serum-free conditioned media; (b) in the cell-free exudates (CFEs); and (c) in serum.

### Results

Lipoxin A4 secreted by resident peritoneal macrophages was decreased by 32% in the diabetic rats compared to the non-diabetic controls, and CMC2.24 in vivo treatment increased the secretion levels of the Lipoxin A4 by 12.7% in resident peritoneal macrophages (FIG. 11). In addition, there was no statistically significant difference in Lipoxin A4 levels between normal rats and diabetic rats treated with CMC2.24. CMC2.24 also increased the levels of the Lipoxin A4 in resident peritoneal cell-free exudates by 80.6%, and increased Lipoxin A4 in rat serum by 15.5% (FIG. 12).

Diabetes in rats modulates inflammatory cell activity in peritoneal exudates, and these abnormalities appear to be “normalized” by oral administration of CMC2.24.

### Example 4. CMC2.24 Increases Lipoxin B4 Levels

### Experimental Details

Adult rats were made diabetic by I.V. injection of streptozotocin; non-diabetic rats served as controls (n=6 rats per group; all groups). Half of the diabetics (blood glucose >500 mg/dl) were orally administered CMC2.24 (30 mg/kg) once per day for 3 weeks; untreated diabetics (D) and N rats received vehicle alone. PEs were collected at time-0 (ie, before thioglycollate injection into peritoneal cavity). Resident macrophages were isolated from PEs, then incubated in cell culture for 18 hours (370 C; 95% air/5% CO₂ atmosphere). Lipoxin B4 levels were measured by ELISA (a) in cell culture serum-free conditioned media; (b) in the cell-free exudates (CFEs); and (c) in serum.

### Results

Lipoxin B4 secreted by resident peritoneal macrophages is decreased in the diabetic rats compared to the non-diabetic controls, and CMC2.24 in vivo treatment increased the secretion levels of Lipoxin B4 by 12.7% in resident peritoneal macrophages (FIG. 11). There is no statistically significant difference in Lipoxin B4 levels between normal rats and diabetic rats treated with CMC2.24). CMC2.24 also increases the levels of the Lipoxin B4 in resident peritoneal cell-free exudates and increases Lipoxin B4 in rat serum.

### Example 5. CMC2.24 Increases Resolvin Levels

### Experimental Details

Adult rats were induced to be type I diabetic by I.V. injection of streptozotocin (70 mg/kg). Non-diabetic rats served as controls. 30 mg/kg of CMC 2.24 was administered daily by oral gavage to STZ-diabetic rats for three weeks. The control diabetic rats received vehicle alone. Resident PE were collected from normal and diabetic rats and resolvin secretion is measured. Resolvin levels are also measured in the rat serum

### Results

CMC2.24 significantly increases the secretion levels of one or more resolvins in resident peritoneal macrophages and in resident peritoneal fluid. CMC2.24 also increases the levels of one or more resolvins in rat serum.

### Example 6. CMC2.24 Increases Lipoxin, Resolvin and Cytokine Levels in a Subject

An amount of CMC2.24 is administered to a subject. The amount of the compound is effective increase production of the one or more lipoxins in the subject. The amount of the compound is effective to increase production of the one or more lipoxins in the subject and one or more resolvins. The amount of the compound is effective to increase production of the one or more lipoxins in the subject, one or more resolvins and one or more anti-inflammatory cytokines in the subject.

### Example 7. CMC2.24 Increases Lipoxin, Resolvin and Cytokine Levels in Subjects Afflicted Inflammatory Disease

An amount of CMC2.24 is administered to a subject afflicted with an inflammatory disease associated with decreased levels of one or more lipoxins. The amount of the compound is effective to treat the subject by inducing production of the one or more lipoxins in the subject. The amount of the compound is effective to treat the subject by inducing production of the one or more lipoxins in the subject and one or more resolvins. The amount of the compound is effective to treat the subject by inducing production of the one or more lipoxins in the subject, one or more resolvins and one or more anti-inflammatory cytokines in the subject.

### Example 9. CMC2.24 Increases Lipoxin, Resolvin and Cytokine Levels in Subjects Afflicted with Inflammatory Dowel Disease

An amount of CMC2.24 is administered to a subject afflicted with inflammatory bowel disease. The amount of the compound is effective to treat the subject by inducing production of the one or more lipoxins in the subject. The amount of the compound is effective to treat the subject by inducing production of the one or more lipoxins in the subject and one or more resolvins. The amount of the compound is effective to treat the subject by inducing production of the one or more lipoxins in the subject, one or more resolvins and one or more anti-inflammatory cytokines in the subject.

### Example 9. CMC2.24 Increases Lipoxin, Resolvin and Cytokine Levels in Subjects Afflicted with Asthma

An amount of CMC2.24 is administered to a subject afflicted with asthma. The amount of the compound is effective to treat the subject by inducing production of the one or more lipoxins in the subject. The amount of the compound is effective to treat the subject by inducing production of the one or more lipoxins in the subject and one or more resolvins. The amount of the compound is effective to treat the subject by inducing production of the one or more lipoxins in the subject, one or more resolvins and one or more anti-inflammatory cytokines in the subject.

### Example 10. CMC2.24 Increases Lipoxin, Resolvin and Cytokine Levels in Subjects Afflicted with Cystic Fibrosis

An amount of CMC2.24 is administered to a subject afflicted with cystic fibrosis. The amount of the compound is effective to treat the subject by inducing production of the one or more lipoxins in the subject. The amount of the compound is effective to treat the subject by inducing production of the one or more lipoxins in the subject and one or more resolvins. The amount of the compound is effective to treat the subject by inducing production of the one or more lipoxins in the subject, one or more resolvins and one or more anti-inflammatory cytokines in the subject.

### Example 11. CMC2.24 Increases Lipoxin, Resolvin and Cytokine Levels in Subjects Afflicted with Rheumatoid Arthritis

An amount of CMC2.24 is administered to a subject afflicted with rheumatoid arthritis. The amount of the compound is effective to treat the subject by inducing production of the one or more lipoxins in the subject. The amount of the compound is effective to treat the subject by inducing production of the one or more lipoxins in the subject and one or more resolvins. The amount of the compound is effective to treat the subject by inducing production of the one or more lipoxins in the subject, one or more resolvins and one or more anti-inflammatory cytokines in the subject.

### Example 12. CMC2 0.24 Increases Lipoxin, Resolvin and Cytokine Levels in Subjects Afflicted with Chronic Obstructive Pulmonary Disease

An amount of CMC2.24 is administered to a subject afflicted with chronic obstructive pulmonary disease. The amount of the compound is effective to treat the subject by inducing production of the one or more lipoxins in the subject. The amount of the compound is effective to treat the subject by inducing production of the one or more lipoxins in the subject and one or more resolvins. The amount of the compound is effective to treat the subject by inducing production of the one or more lipoxins in the subject, one or more resolvins and one or more anti-inflammatory cytokines in the subject.

### Example 13. Chronic Obstructive Pulmonary Disease (COPD)

Nice: For the present study, age matched male and female wild-type C57BL/6 mice (purchased from Jackson laboratory), were used. Transgenic mice used in this study were bred in the animal core facility at SUNY Upstate Medical University under pathogen-free conditions. All animal experiments were conducted in accordance with the Institutional Animal Care and Use Committee guidelines of SUNY Upstate Medical University and the National Institutes of Health guidelines on the use of laboratory animals. Mice were divided into five groups: the control group, COPD group, COPD plus PM_{2.5}, COPD plus CMC2.24 and COPD plus PM_{2.5} and CMC2.24. All protocols related to animal experiments were approved by the institutional animal care and use committee of SUNY Upstate Medical University. Experiments were performed according to the National Institutes of Health guidelines and ARRIVE guidelines on the use of laboratory animals.

Elastase and LPS exposure: A total of 180 male and 180 female WT mice (8-12 weeks old) were used for all experiments. Experiments were performed in triplicate for each group of age- and gender-matched mice. Animals were exposed by the intranasal route to 10 μl saline containing 1.2 units of porcine pancreatic elastase (Elastin Products, Owensville, MO) on Tuesday and 10 μl saline containing 7 μg (˜70 endotoxin units) of LPS from Escherichia coli 026:B6 (Sigma-Aldrich, St. Louis, MO) on Friday of each week for four consecutive weeks.

Chemically modified curcumin (CMC2.24): Chemically-modified curcumin (CMC2.24) is a phenylamino carbonyl curcumin that has improved zinc-binding structure. It is triketonic in contrast to the diketonic active site on natural curcumin compounds, and has shown evidence of efficacy in vitro, in cell culture, and in animal models of chronic inflammatory and other diseases. 3 mg of CMC2.24 powder were dissolved in 1 mL suspension of 2% Carboxymethyl cellulose vehicle for the daily oral administration of CMC2.24 (40 mg/kg). Vehicle alone was administered to the control group. Both CMC2.24 and vehicle control were administered once daily over the 7-day protocol by gavage (Zhang, Y. et al. 2010; Elburki, M. S. et al. 2014).

Animal Surgery and administration of PM_{2.5}: After 7 days from the last LPS dose mice in the COPD and sham groups were anesthetized by intraperitoneal injection with a combination of ketamine (90 mg/kg) and xylazine (10 mg/kg) (i.e. 0.1 ml/130 g animal weight). The intensity of anesthesia by toe pinching using tweezers was monitored. Mice were positioned on a taut string secured at one end, hanging from their incisors. A longitudinal incision was made in the midline of the neck; separate the thyroid gland lobes to expose the trachea. 50 μl saline containing 125 μg of PM_{2.5} was injected by intratracheal injection. Then, the incision was stapled closed. This was followed by giving 1 mg of CMC2.24 in 300 μl of vehicle (Carboxymethyl Cellulose) daily by gavage for seven days in a subgroup of mice. Mice were returned to cages at the end of the surgical procedures where access to water and food is available. The mice were injected with buprenorphine (0.05 mg per kg body weight s.c.) for postoperative analgesia. Mice were placed back in cages in a temperature-controlled room (22° C.) with 12-h light and dark cycles and monitored every 6 h.

### Behavioral Testing

Inverted Screen Test: It is a test of muscle strength using all four limbs. The inverted screen test was devised by Kondziela and published it in 1964. For the inverted screen test, the mice were placed on a metal grid screen (11×18 inch) with separate compartments. After placement, the mice were allowed time to grip the grid before it was inverted 60 cm over a Styrofoam container. Latency to fall was recorded up to 120 s, at which point mice were removed from the apparatus and returned to the home cage. Three independent trials were conducted approximately 15 min apart on the day of testing, and data from all three trials were averaged together. The scores were graded as follows (1) 0-30 seconds, (2) 31-60 seconds, (3) 61-90 seconds (4) 90-120 seconds (Deacon, R. M. 2013; Frederick, A. L. et al. 2012; Guenther, K. et al. 2001).

Tissue Collection: After anesthetizing mice with ketamine: xylazine 100 mg/10 mg, a large abdominal incision was made and the intestine was turned to the left side the inferior vena cava and Aorta were cut using iris scissors and the animal was left to bleed. After death of the mouse, various tissues were harvested from the mice including lung, liver, spleen, kidney and intestine. Tissues were wrapped in a labeled aluminum foil, snap frozen in liquid nitrogen and kept in −80° C.

Lung Histopathology: Randomly selected lungs were slowly inflated with 0.5 ml of 10% formalin and then completely immersed in 10% formalin. Specimens were embedded in paraffin and 5 μm sections cut. Slides were stained for standard light microscopy using hematoxylin and eosin. Periodic acid-Schiff (PAS) staining were used for detecting the inflammatory changes of the lung tissue and goblet cell hyperplasia.

Lung Injury scoring system: The lung injury scores were calculated using the method described by Matute-Bello and colleagues (Matute-Bello, G. et al. 2011). Lung sections were scored using a 0-2 scale by an investigator for the presence of (A) alveolar and (B) interstitial neutrophils, (C) alveolar hyaline membranes, (D) proteinaceous debris filling the airspaces and (E) Alveolar septal thickening were scored in twenty high power fields; the resulting scores were calculated by the following formula: Score=[(20×A)+(14×B)+(7×C)+(7×D)+(2×E)]/(number of fields×100).

Morphometry—Air Space Enlargement: In an effort to quantitate alveolar air space enlargement, the Mean Linear Intercept (MLI) was implemented. The Mean Linear Intercept method is a stereological technique that allows for the measurement of the acinar air space complex, including both alveoli and alveolar ducts combined. It provides a meaningful estimate of alveolar airspace size. Using NIS-Elements' Software, digitalized images at 200× magnification were taken on the Nikon Eclipse TE2000-U microscope and then printed for each sample. A guard frame was then introduced within each of the images. Afterwards, seven equally spaced lines were then drawn within the guard zone, and directly measured by manual use of a ruler. Starting from the left, the line was scanned for any intersections with the alveolar walls and measured until the following intersection with the alveolar surface on the right. Alveolar Surfaces that extend beyond the guard frame on the left side are not included in the calculation, but those on the right are included. The intercept lengths were summed and divided by the total amount of intercept lengths made, deriving the MLI parameter. The MLI's were then compared with one another to determine if there is a decrease in alveolar walls due to emphysema (Knudsen, L. et al. 2010).

Apoptotic cell determination by TUNEL assay: Unstained lung sections from different groups of mice were incubated at 60° C. for 20 min. The sections were deparaffinized in xylene twice and treated with graded series of alcohol (100%, 90%, 80%, and 70% ethanol/ddH2O) and rinsed in phosphate-buffered saline (pH 7.5). Apoptotic cells were detected using deoxynucleotidyl transferase-mediated dUTP nick-end labeling (TUNEL) kit (Roche) following the manufacturer's instructions. Apoptotic (TUNEL-positive) cells were quantified in 20 randomly chosen fields at ×400 magnification.

BAL fluid preparation: Bronchoalveolar lavage fluid (BALF) was obtained using 1.0 ml of saline. After the mouse is exsanguinated the trachea is cannulated with a tracheal cannula. BALF is centrifuged at 250 rcf for 10 minutes and the supernatant is kept in −20° C., the pellet is re-suspended in 1 ml saline. The sample is centrifuged in the Hettich ROTOFIX 32A Benchtop Centrifuge at 1000 rpm for 3 minutes to fix macrophages to a glass slide. Slides were stained for standard light microscopy using the Protocol HEMA-3 cell staining kit (Fisher Diagnostics; Middletown, VA) and were examined by Nikon Eclipse TE2000-U microscope. To determine the percentage of macrophages, and neutrophils we counted 300 cells in random high-power fields and differential cell count was calculated for each sample.

Gelatin Zymography: Gelatin zymography was performed to quantify the MMP-2 and MMP-9 activities in the BAL fluid. An aliquot (25 μl) of the BAL fluid supernatant was loaded onto a 10% polyacrylamide gel containing 0.1% (wt/vol) gelatin under non-reducing conditions. After electrophoresis, the gel was washed with renaturing buffer (2.5% Triton X-100), for 30 min. The renaturing buffer was removed and 100 mL of developing buffer (40 mM Tris, 200 mM NaCl, and 10 mM CaCl₂; pH 7.5) was added to the gel and incubate for 30 minutes at room temperature with gentle agitation. The gel was then incubated in a fresh 100 mL of developing buffer at 37° C. for 24 hours. The gel was then stained in 0.05% (wt/vol) Coomassie Brilliant Blue, 30% (vol/vol) methanol, and 10% (vol/vol) acetic acid for 1 h, and destained for 3 h.

To quantify the MMP-12 level in the BAL fluid we used a 12% polyacrylamide gel containing 0.05% (wt/vol) casein following the same protocol. Densitometry was carried out using ImageJ software version 1.48 (Wayne Rasband, National Institutes of Health, Bethesda, MA).

Cytokine determination in the BALF: The concentrations of IL-6 and TNF-α in the BALF were measured using commercially available murine enzyme-linked immunosorbent assay (ELISA) kits in accordance with the manufacturer's instructions (Life Technologies, Frederick, MD) (Liu, J. et al. 2015).

Analysis of oxidative stress in the BALF: The level of 8-Isoprostane in BALF as a marker for oxidative stress were analyzed using a commercial ELISA kit according to manufacturer's instructions (Eagle Biosciences, Inc.).

Determination of total protein concentration and Western Blot analysis: The total protein concentrations of BAL were determined using the BCA micro assay kit (Thermo). Total protein (80 μg) was resolved by reducing (for SP-A and SP-D) 12% SDS-polyacrylamide gel electrophoresis and then transferred electrophoretically at 60 mA onto nitrocellulose membranes at 4° C. overnight (Bio-Rad, USA). After the samples were blocked in 3% non-fat milk in Tris-buffered saline, immunoblotting was detected using a primary antibody against SP-A (1:1000), and SP-D (a rabbit anti-mouse SP-D antibody at 1:3000 dilution), and an anti-rabbit secondary antibody conjugated with horseradish peroxidase. Immunoproducts were detected using Pierce ECL Western Blotting Substrate (Thermo Scientific) and the blots were exposed to X-film (Pierce Biochemicals, FL). Human BAL and WT mouse BAL were used as positive controls for SP-A and, SP-D separately.

Statistical analysis: Experimental data were analyzed by SigmaStat 3.5 software (Systat Software, Inc., San Jose, CA) and presented as means±standard error. Two-group comparisons were performed using Student's t test. A P value of <0.05 was considered to be statistically significant.

### Results

### COPD Mouse Model:

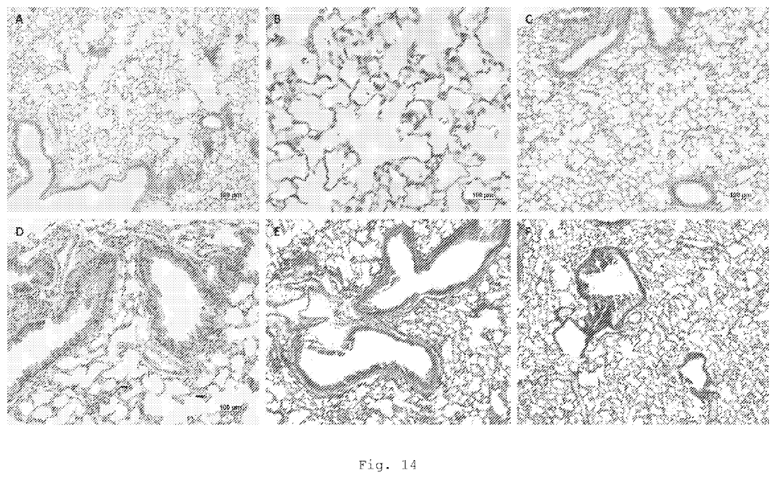

Histological examination of the lungs: To induce COPD features in a mouse model elastase and LPS was administered to the mice for four weeks in the manner detailed in the methodology section. After Seven days from the final treatment with elastase/LPS, a group of mice was euthanized for histological examination; the lung was inflated by 0.5 ml of 10% formalin, fixed in formalin and embedded in paraffin. H&E stained sections showed alveolar destruction, which resulted in enlarged air spaces, indicating emphysematous change (FIGS. 14 A & B). The second group of mice was given 50 μl PM_{2.5} intratracheally. The third group of mice was given 50 μl PM_{2.5} intratracheally followed by 100 μg CMC2.24 by gavage for seven days.

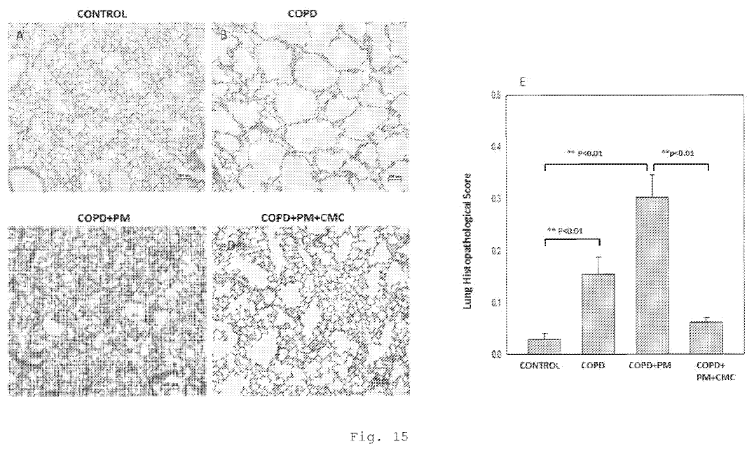

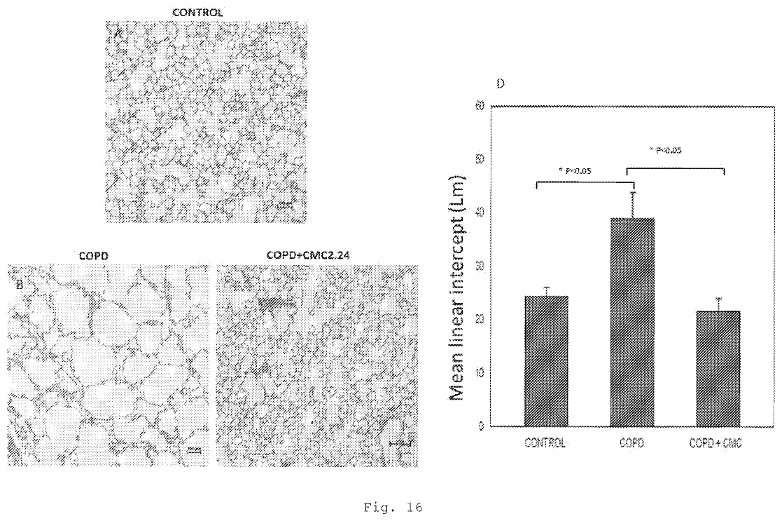

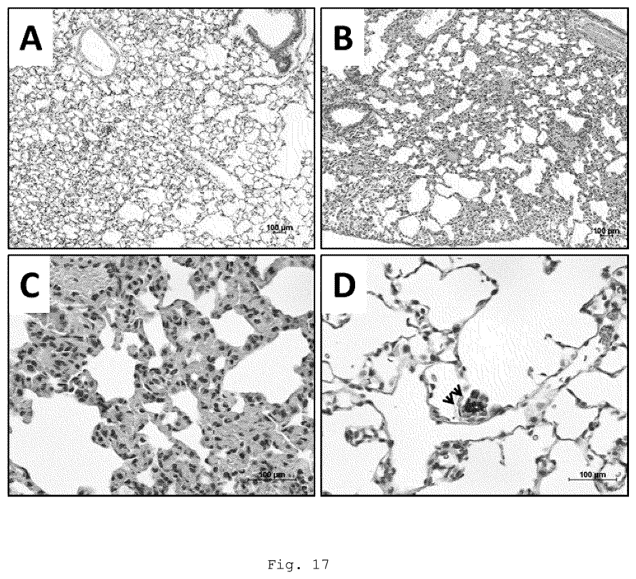

Elastase/LPS-treated mice showed widespread inflammatory changes in the lung. Aggregations of neutrophils and mononuclear inflammatory cells were observed both in the perivascular and peribronchiolar spaces (FIGS. 14 C & D). Increased numbers of PAS-positive cells in both the large and small airways was also observed (FIGS. 14 E& F). The histopathologic score of lung injury significantly increased in COPD mice and showed further increase after administration of PM_{2.5} (FIGS. 15A, 15B & 15C, P<0.01). CMC2.24 treatment of COPD and PM_{2.5} exposed mice was associated with a significant reduction in lung injury histopathologic score (FIG. 15D, P<0.01).

Morphometry: The mean linear intercept (MLI), or chord length was calculated as a measure of the acinar air space complex, that includes both alveoli and alveolar ducts combined, using a light microscope at a magnification of ×200. Average chord length in control mice was found to be 33 μM (FIG. 16; Panel A ) which was significantly increased to 54 μM, (P<0.05) in COPD mice treated with Elastase/LPS showing alveolar destruction, and enlarged air spaces, both indicating emphysematous change (FIG. 16; Panel B). CMC 2.24 treatment of COPD mice was associated with a significant reduction in alveolar chord length (FIG. 16; Panel C). In CMC 2.24-treated mice this was found to be 35 μM (p<0.05). The average chord length was obtained for a group of COPD mice exposed to PM_{2.5} (54 μM), and although it was not significantly different from the group of COPD mice, treatment of this group with CMC 2.24 resulted in significant reduction of chord length (p<0.01). These data suggest that CMC 2.24 treatment prevented further progression of emphysema after exposure to PM_{2.5} and actually stimulated the regeneration of degraded alveoli.

### Effects of PM_{2.5} on COPD Model:

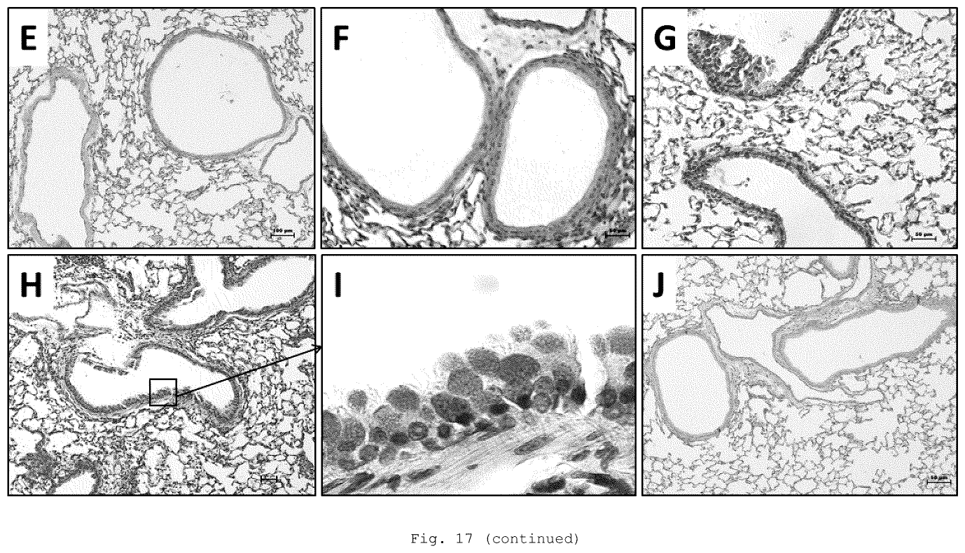

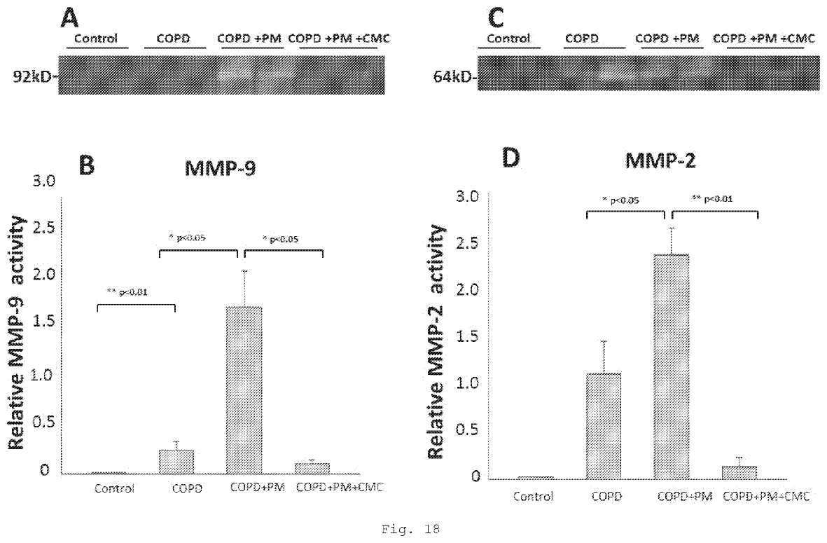

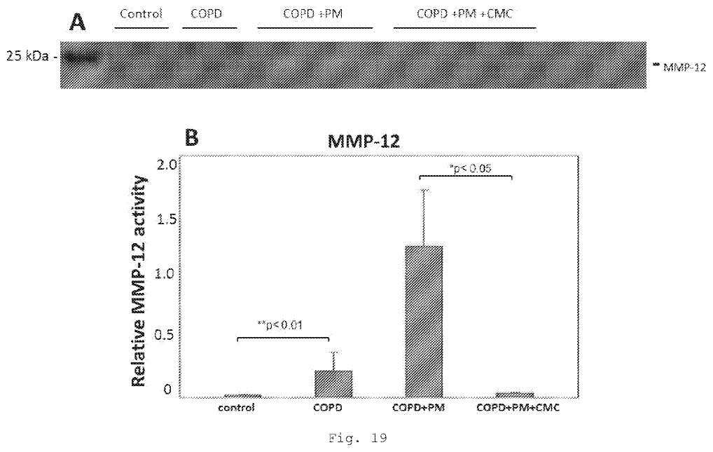

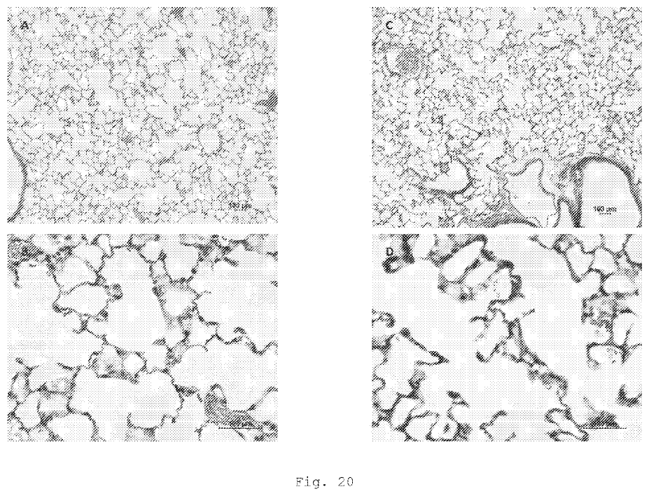

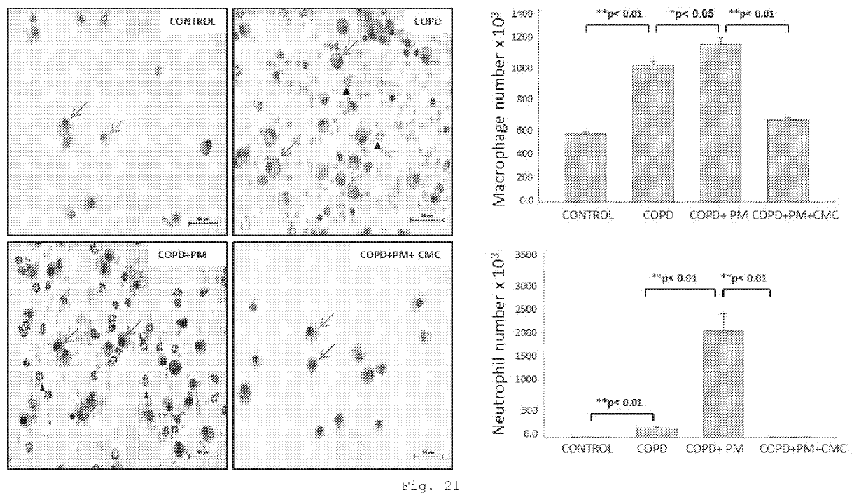

Histological Changes: Severe inflammatory changes were observed in the lung parenchyma of the elastase/LPS-exposed mice after intratracheal injection of 125 μg of PM_{2.5} (FIGS. 17A and B), with widespread neutrophilic inflammation (FIG. 17C), including the airway lumina and alveoli. The inflammation persisted up to 7 days post PM_{2.5} administration. Many PM_{2.5} particles were observed inside inflammatory macrophage-like cells (FIG. 17D).

Elastase/LPS-exposed mice showed more PAS-positive material than the control mice in both large and small airways (FIGS. 17; Panels E, F and G). PAS-positive cells increased in number after PM_{2.5} administration to elastase/LPS-exposed mice (FIGS. 5; Panels H and I), and increased goblet cell metaplasia in the small airways was also observed. CMC 2.24 treatment prevented goblet cell metaplasia in PM2.5 challenged mice (FIG. 5; Panel J).

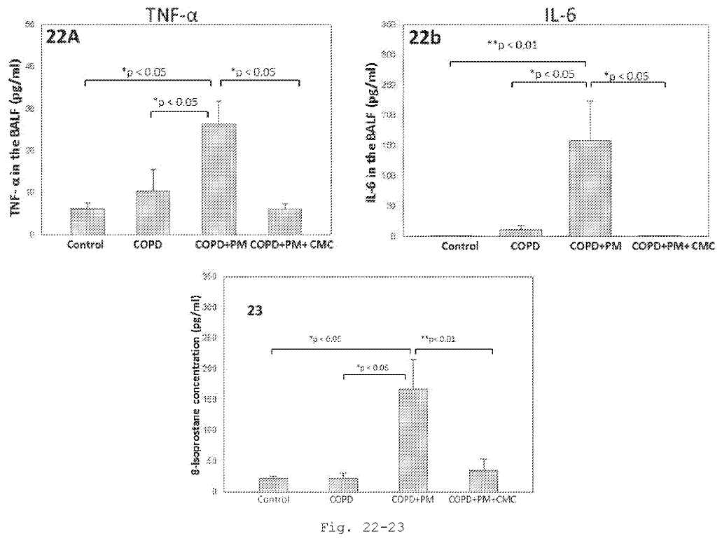

Effect of PM_{2.5} on MMP-2, MMP-9 and MMP-12 in BALF supernatant from COPD mouse model: Gelatin zymography revealed significantly increased activity of MMP-9 in BALF supernatants from COPD mice in comparison with control mice (FIGS. 6; Panels A and B: P<0.01) and a further increase in the BALF from COPD mice exposed to PM_{2.5}. This activity was significantly inhibited by CMC 2.24 treatment (back to control levels) in mice exposed to PM_{2.5} (FIGS. 18; Panels A and B: P<0.05).

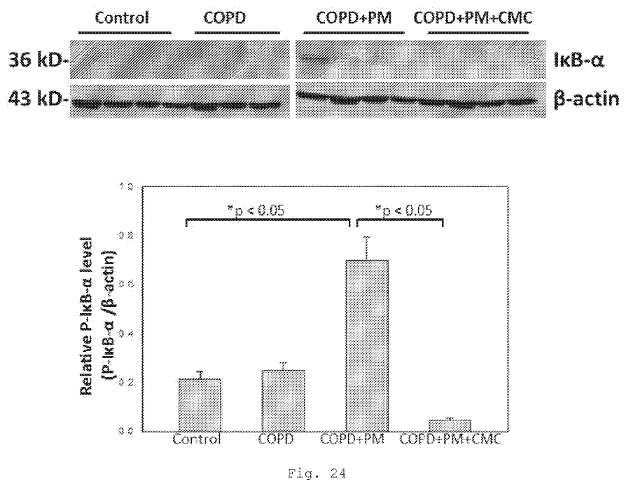

The activity of MMP-2 increased significantly after the administration of PM_{2.5} to COPD mice (FIGS. 18; Panels C and D: P<0.05). This activity was also significantly inhibited by CMC 2.24 treatment in mice exposed to PM_{2.5} (FIGS. 18; Panels C and D: P<0.01). With regard to casein zymography, the activity of MMP-12 was significantly elevated in COPD mice, and further elevated in COPD+PM2.5 mice compared to control mice (FIGS. 19; Panels A and B: P<0.01). This activity was also significantly inhibited by CMC 2.24 in mice exposed to PM_{2.5} and returned the elevated MMP-12 to essentially “control” levels (FIGS. 19: Panels A and B: P<0.05).

Administration of CMC2.24 protects COPD mice model from the Development of marked inflammatory changes in Response to PM_{2.5}: In order to determine the efficacy of treatment with CMC2.24 on the development of severe inflammatory response in COPD mouse model, mice were exposed to PM_{2.5} and were either treated with the vehicle or treated with 100 μg CMC2.24 by gavage for 7 days. The effect on histological picture, cell count and MMP-2, MMP-9 and MMP-12 activities was then evaluated.

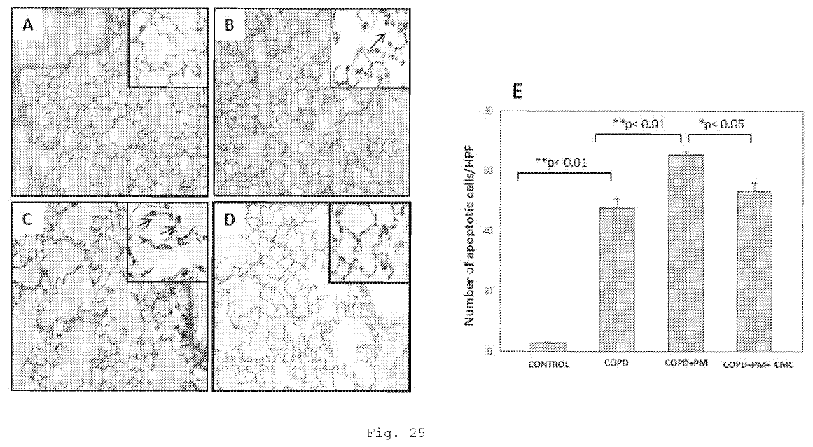

Effects of CMC2.24 on COPD mice exposed to PM_{2.5}: Mice exposed to 125 μg of PM_{2.5} showed marked and significant influx of inflammatory cells in both the lung tissue and BAL fluid up to seven days post exposure. The oral administration of 100 μg of CMC2.24 daily for 7 days to COPD mice exposed to PM_{2.5} protected the mice from developing the inflammatory changes seen in PM_{2.5} exposed mice. Lung tissue looked almost normal (FIG. 20).

BAL cell counts revealed that CMC2.24 significantly reduced the increase in total number of inflammatory cells in COPD mice exposed to PM_{2.5}. Mice exposed to PM_{2.5} looked less active and lethargic, while mice treated with CMC2.24 had normal activity.

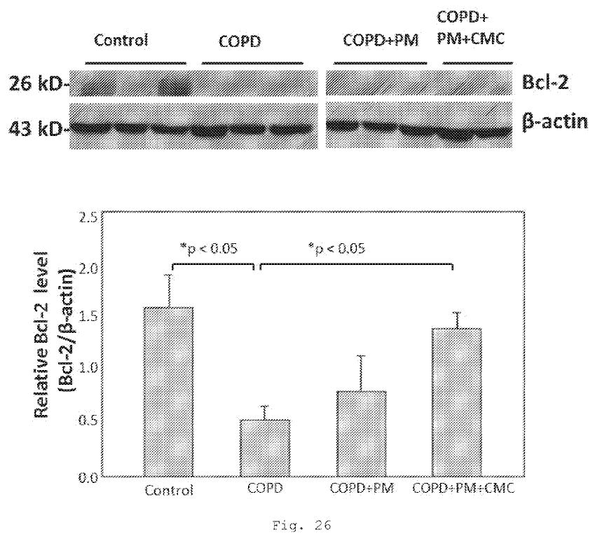

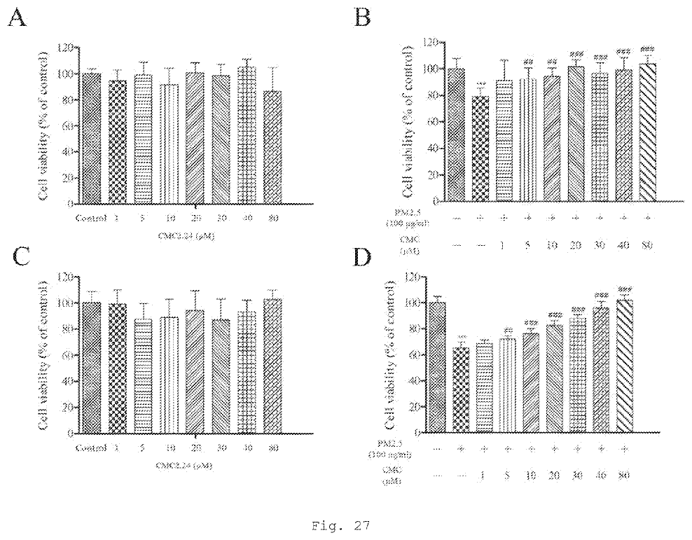

Cellular analysis of DAL: Bronchoalveolar lavage fluids (BALF) were centrifuged at 250×g for 10 minutes and the pellets were resuspended in 1 ml saline. This suspension (200 μl) was used to prepare the slides for the cytological evaluation described above. To determine the percentages of macrophages, and neutrophils, 300 cells were counted in random high-power fields and the differential cell count was calculated for each sample (De Brauwer, E. I. et al. 2002). Cytological analysis of bronchoalveolar lavage (BAL) fluid showed a significant increase in the percentage of neutrophils in COPD-mice exposed to PM_{2.5} compared with COPD-mice (FIG. 21). However, COPD-mice exposed to PM_{2.5} and treated with CMC 2.24 were protected against the increase in inflammatory cell numbers. The number of macrophages and neutrophils was significantly increased in elastase/LPS-treated COPD mice compared with controls.

### Inflammatory Cytokines

The levels of TNF-α and IL-6 in BAL fluid were determined by ELISA. This showed a significant increase in the level of TNF-α in PM_{2.5} challenged mice (p<0.05). The level of TNF-α showed significant decrease (p<0.05) in PM_{2.5} challenged mice treated with CMC 2.24 (FIG. 22; Panel A). The level of a long-lived proinflammatory cytokine (IL-6) also increased significantly in PM_{2.5}-challenged mice (p<0.01) but decreased substantially in PM_{2.5} challenged mice treated with CMC 2.24 (P<0.05) (FIG. 22; Panel B).

### Oxidative Stress Measurement

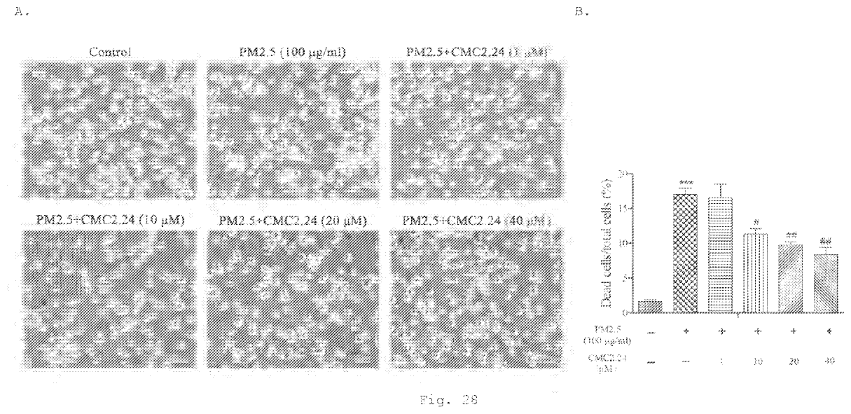

The levels of 8-Isoprostane in BALF as a marker for oxidative stress were measured using the 8-Isoprostane ELISA kit (Eagle Biosciences, Inc.). This showed a significant increase in the levels of 8-Isoprostane in PM_{2.5} challenged mice (FIG. 23: p<0.05). The levels of 8-Isoprostane decreased significantly in PM_{2.5} challenged mice which had been treated with CMC 2.24 (FIG. 10, p<0.01).

### Phosphorylated-IκB-α Levels in the Lung

Curcumin was found to down-regulate NF-κB and phosphorylated IκB-α which are responsible for modulating many genes involved in inflammation and oncogenesis (Shishodia S, et al. 2003).

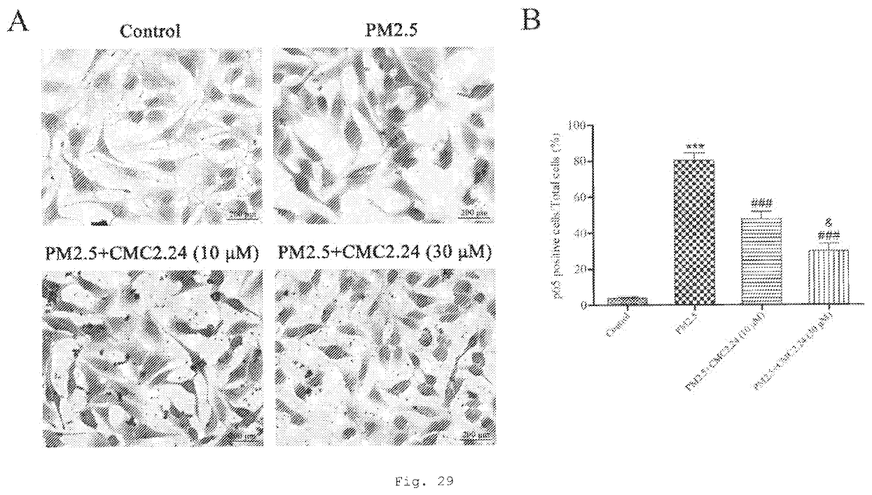

Western blot was used to measure the level of phosphorylated IκB-α (p-IκB-α) in different study groups. Our results showed significantly increased level of p-IκB-α in PM_{2.5} challenged mice compared with the control mice (FIG. 11, p<0.05). The levels of p-IκB-α significantly decreased in PM_{2.5} challenged mice treated with CMC 2.24 (FIG. 24, p<0.05).

### Lung Cell Apoptosis

To study the effect of COPD, PM_{2.5} and CMC 2.24 treatment on lung cell apoptosis TUNEL assay was used unstained lung histology slides and western blot analysis for apoptosis related protein Bcl-2 expression.

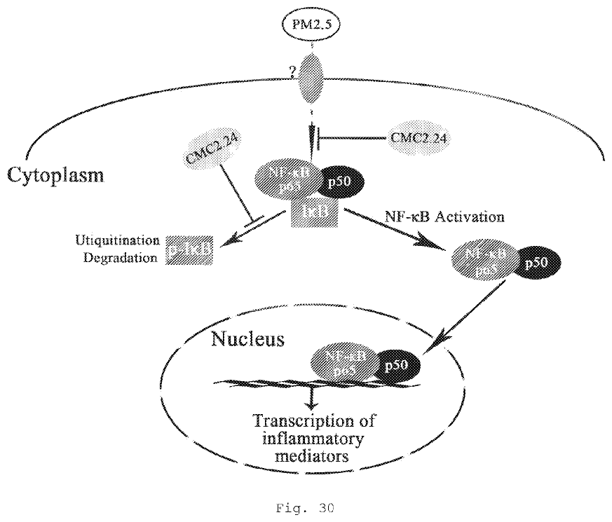

Apoptosis Analysis by TUNEL Lung-tissue slides stained by the TUNEL method to detect apoptotic cells in the control, COPD, PM_{2.5}, and PM_{2.5}+CMC 2.24 groups revealed a significant increase in the number of apoptotic cells in mice challenged with PM_{2.5} in comparison with control mice (FIG. 25, p<0.01), apoptotic cells nuclei look dark brown. Such mice challenged with PM_{2.5} but treated with CMC 2.24 showed a significant reduction in the number of apoptotic cells in comparison with the untreated group (FIG. 25, p<0.05) healthy cells nuclei look blue.

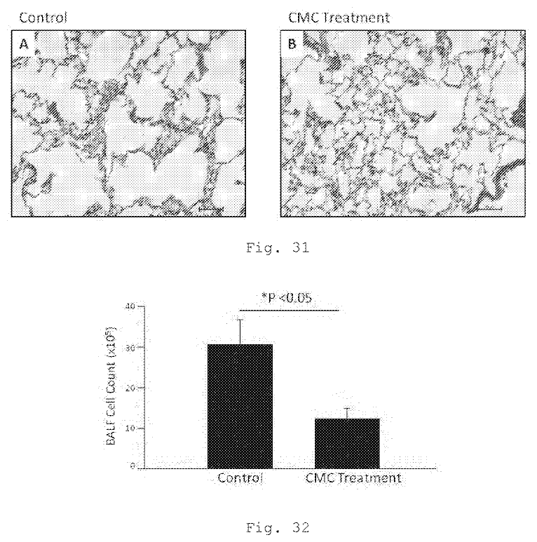

Western blot analysis for apoptosis related protein Bcl-2 expression. Bcl-2 is a negative apoptosis marker that is higher in normal cells and decrease in apoptotic cells. Western blot for Bcl-2 revealed significantly lower levels in COPD compared to control mice (FIG. 26, p<0.05). Those mice challenged with PM_{2.5} but treated with CMC 2.24 showed a significantly higher level of Bcl-2 (FIG. 26, p<0.05).

### Behavioral Testing and Muscle Strength

The COPD mice challenged with PM_{2.5} showed less physical activity, accompanied with sluggish responses and less interest in grooming their fur, all of which usually denotes mouse distress. In contrast, mice treated with CMC 2.24 showed marked improvement in overall activity, and displayed clean-groomed fur. These observations were quantified by measuring muscle strength in treated and untreated groups using the inverted screen test described in the methods section. The results of behavioral testing showed that CMC 2.24 treatment improved mouse muscle strength, especially for the PM_{2.5}-exposed COPD-mice and the results were statistically significant.

### Example 14. CMC2.24 Improves Cell Variability and Decreases Inflammation in Lung Epithelial Cells and Macrophages Exposed to Air Pollutant

Human lung epithelial cell line (A549) and primary alveolar macrophage call culture: Human lung epithelial cell line (A549, ATCC #CCL-185) was purchased from ATCC(Manassas, VA); and primary alveolar macrophages were prepared from healthy adult animals (swine). A549 cells and primary alveolar macrophages were cultured in RPMI Media 1640 medium supplemented with 10% (v/v) FBS, 1% (v/v) L-glutamine (200 mM) and 1% (v/v) Antibiotic-Antimycotic antibiotics at 37° C. in a humidified 5% CO2 incubator.

CMC 2.24 treatment and PM_{2.5} exposure: The cells were subcultured when cells were grown to about 70% confluency. After 24 h of subculture, the cells were treated with a range of concentrations of CMC2.24 from 0 to 80 NM of CMC 2.24 (final concentrations in the media) for 0.5 h prior to using 100 μg/ml PM_{2.5} exposure. Cell viability and death were examined for 24 h after PM2.5 treatment.

Analysis of cell viability by CCK-8 assay: In order to study the effect of CMC2.24 in A549 cells and primary alveolar macrophages after PM_{2.5} exposure, cell viability was determined using Cell Counting Kit (CCK)-8 kit (Sigma-Aldrich, MO, USA) according to the manufacturer's instructions. A549 cells (0.5×10⁴/well) and primary macrophages (1×10⁴/well) were cultured in 96-well plates for 24 h and 6 h, respectively; the cells were then exposed to various concentrations of CMC 2.24 (i.e. 0, 1, 5, 10, 20, 30, 40, and 80 μM of CMC 2.24) in the presence or absence of 100 μg/ml PM_{2.5} in the media for 24 h. Each well was added 10 μl of 10% CCK-8 solution and incubated for 1 to 4 h. Then optical density value was measured at 450 nm using a microplate reader (Multiskan Ascent, Thermo Lab systems). Relative cell viability was calculated as percentage of the control group.

Call death analysis by Trypan blue staining: A549 cells were cultured in 6-well plates and reached about 70% confluency. Cells were treated with a range of concentrations of CMC 2.24 from 0 to 40 μM and then exposed by 100 μg/ml PM_{2.5} in the media for 24 h. After 24 h of treatment, cells were trysinized and collected by a centrifugation at 1000 rpm for 5 min at 4° C. The cells were gently resuspended in 100 μl phosphate-buffered saline (PBS), and 10 μl suspension were mixed with 10 μl of 0.4% (w/v) trypan blue solution for 1 to 3 min. Dead cells were examined using a Nikon Eclipse TE 2000-U microscope (Nikon Instruments Inc., Melville, NY) (×200). Dead cells were shown blue color. Ratio of dead cells/total cells for each group was analyzed among groups.

Immunohistochemical analysis: Immunohistochemical analysis was used for the examination of NF-kB p65 protein expression and nuclear translocation in treated A549 cells. A549 cells were treated with various conditions i.e. PM2.5, PM2.5+CMC 2.24 (10 or 30 μM) for 24 h. The cells were washed with 37° C. PBS twice, fixed with 4% paraformaldehyde for 20 min, permeabilized with 0.5% Triton X-100 buffer (Sigma-Aldrich, MO, USA) at room temperature for 5 min, and then blocked with 5% bovine serum albumin (BSA) at 4° C. for 10 min. The cells were washed with PBS three times at each above step. The cells were then incubated with rabbit anti-p65 (NF-kB) antibody (Santa Cruz Biotech, Dallas, USA; 1:50 dilution) overnight at 4° C. After washing with PBS, cells were incubated with secondary antibody (1:200 dilution) for 1 h. The immunohistochemical reaction was visualized by diaminobenzidine stain kit (Vector, CA). Nuclei were counter-stained with haematoxylin for 1 min, and images were visualized by a phase-contrast microscopy (×20 C). The ratio of NF-kB p65 nuclear positive cells/total cells for each group was determined and statistically analyzed.

Statistical analysis: Experimental data were analyzed by SigmaStat 3.5 software (Systat Software, Inc., San Jose, CA) and presented as means±standard error. Data were compared using the Student's t test or ANOVA. For all comparison, a p value of <0.05 was considered to be statistically significant.

### Results

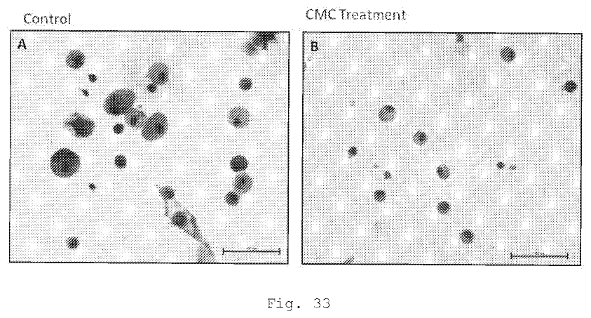

Effect of CMC2.24 on cell viability: The results from CCK-8 assay indicated that CMC2.24 treatment did not influence cell viability on A549 cells and primary alveolar macrophages at a range of concentrations from 1 to 80 μM of CMC 2.24 for 24 h (FIGS. 27A and C). With the treatment of PM2.5 (100 μg/ml) in the media A549 cells and primary macrophages showed decreased cell viability (p<0.001) for 24 h compared to control group (FIGS. 27B and D). With the pretreatment of CMC 2.24 (the concentration with more than 5 μM) the cell viability of both A549 and alveolar macrophages showed significant improvement compared to without CMC 2.24 treatment. The improvement of cell viability showed CMC 2.24-dose-dependent effects in the alveolar macrophages from 5 to 80 μM of CMC 2.24.

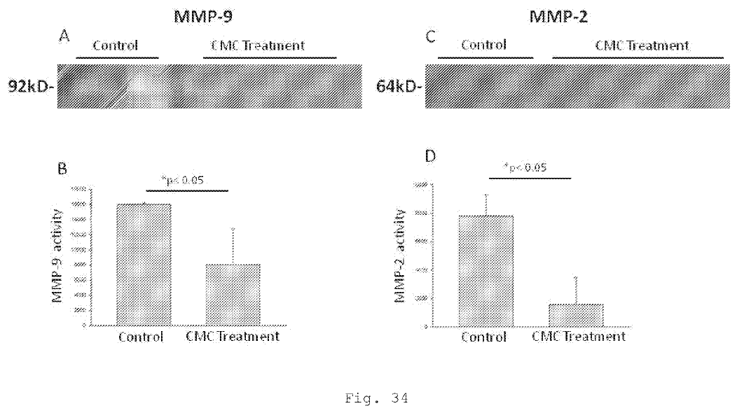

Effect of CMC2.24 on PM_{2.5}-induced A549 cell death: To examine the effect of CMC 2.24 on PM_{2.5}-induced A549 cells death, treated A549 cells with CMC 2.24 and PM2.5 were examined using trypan blue staining method. Dead cells were stained with blue. As shown in FIG. 28, the ratio of dead cells/total cells was increased significantly in the PM_{2.5} group as compared to the control (***p<0.001). However, the ratio of dead cells/total cells reduced significantly in the groups with the treatment of CMC 2.24 from 10 to 40 μM compared with the PM_{2.5} group (^{#}p<0.05, and ^{##}p<0.01) and showed a dosage-dependent effects for cell survivals (FIG. 28B).

Effect of CMC2.24 on NF-κB p65 expression and nuclear translocation on PM_{2.5}-treated A549 cells: To explore the effect of CMC2.24 on NF-κB signaling activation of PM2.5-treated A549 cells, NF-κB p65 expression and nuclear translocation were examined using immunohistcchemistry. As shown in FIG. 29, the results showed that NF-κB p65 expression and nuclear translocation in the PM_{2.5}-treated group were significantly increased compared to the control group (**p<0.001). However, compared to the PM_{2.5}-treated group, the NF-κB p65 expression and nuclear translocation were inhibited significantly with the treatment of CMC 2.24 at both 10 and 30 μM in FIG. 3B (^{###}p<0.001).

### Example 15. Therapeutic Effects in Emphysema Model

Mice: SP-D knockout (KO) mice (10 months old and male) with C57BL/6 background were used in this study. SP-D KO mice have been shown to develop an early onset emphysematous phenotype. Emphysematous SP-D KO mice were administrated with CMC 2.24 or vehicle control by oral gavage daily.

Chemically modified curcumin (CMC 2.24) and animal treatment: The chemically-modified curcumin (CMC 2.24) is a phenylaminocarbonyl derivative of curcumin. Three milligrams of CMC 2.24 powder were dissolved in a 1 mL suspension of 2% Carboxymethyl cellulose vehicle for daily oral administration (40 mg/kg of animal body). Vehicle alone was administered to the control group. Both CMC 2.24 and vehicle control groups were administered once daily over the 7-day protocol by oral gavage.

Animal scarification and tissue collection: One day after the last dose of CMC2.24 mice in the control and treatment groups were anesthetized by intraperitoneal injection with a combination of ketamine (90 mg/kg) and xylazine (10 mg/kg; i.e. 0.1 ml/100 g animal weight). The intensity of anesthesia was monitored by means of toe-pinching using tweezers. After insuring that the mouse is deeply anesthetized a large abdominal incision was made and the intestine was turned to the left side of the inferior vena cava and aortas were cut using iris scissors and the animal was left to bleed. After that, various tissues were harvested from the mice including lung, liver, spleen, kidney and intestine. Tissues were wrapped in labeled aluminum foil, snap frozen in liquid nitrogen and kept at −80° C.

BAL fluid preparation and cell analysis: After the mouse was exsanguinated, the trachea was cannulated with a tracheal cannula and 1 ml of saline was used to wash the bronchoalveolar tree and obtain the bronchoalveolar lavage fluid (BALF). BALF was centrifuged at 250×g for 10 minutes, the supernatant was kept at −20° C., and the pellet resuspended in 1 ml saline. The sample was centrifuged in the Hettich ROTOFIX 32A Benchtop Centrifuge at 1000 rpm for 3 minutes to affix macrophages to a glass slide. Slides were stained for standard light microscopy using the Protocol HEMA-3 cell staining kit (Fisher Diagnostics; Middletown, VA) and were examined by means of a Nikon Eclipse TE2000-U microscope.

Lung Histopathology: Selected lungs were slowly inflated with 0.5 ml of 10% formalin and then completely immersed in 10% formalin. Specimens were embedded in paraffin and 5-μM sections were cut. Slide sections were stained for standard light microscopy using hematoxylin and eosin. The lung histology and injurious scores were assessed blindly by two experienced investigators using the method as described by Matute-Bello and colleagues (Matute-Bello et al 2011).

Gelatin Zymography: Gelatin zymography was performed using standard techniques in the densitometric analyses of MMP-2 and MMP-9 activities. An aliquot (24 μl) of the BAL fluid supernatant was loaded under non-reducing conditions onto a 10% polyacrylamide gel containing 0.1% (wt/vol) gelatin. After electrophoresis, the gel was washed with renaturing buffer, for 30 minutes and then in developing buffer for 30 minutes at room temperature with gentle agitation. The gel was then incubated in a fresh 100 mL of developing buffer at 37° C. for 24 hours. It was then stained in 0.05% Coomassie Brilliant Blue, for 1 h and destained for 3 h. To quantify the MMP-12 level in the BAL fluid we used a 12% polyacrylamide gel containing 0.05% (wt/vol) casein following the same protocol. Densitometry was carried out using ImageJ Software Version 1.48 (Wayne Rasband, National Institutes of Health, Bethesda, MD).

Statistical analysis: Experimental data were presented as means±standard error and analyzed by SigmaStat 3.5 software (Systat Software, Inc., San Jose, CA). Data were compared using the Student's t test or ANOVA. For all comparisons, a p value of <0.05 was considered to be statistically significant.

### Results

Treatment with CMC 2.24 attenuated lung inflammation and improved alveolar structure in emphysematous SP-D KO mice: SP-D KO mice develop emphysematous symptom at more than 6 months old stage. In this study about 10-months old mice were used and the lung of these mice have shown all characteristics of emphysema. The emphysematous SP-D KO mice were divided two groups. i.e. CMC 2.24 treatment and Control (vehicle treatment). The emphysematous mice were treated by daily oral gavage of CMC 2.24 (4 C mg/kg of animal body) or vehicle for seven days. The lung histology was examined and scored using the method as described by Matute-Bello and colleagues (Matute-Bello et al 2011). The results indicate that the lungs of untreated mice (control) show alveolar widening denoting emphysema and perivascular mononuclear inflammatory cell infiltration (FIG. 31A), but the lungs of CMC 2.24-treated mice exhibit decreased inflammatory cell infiltration and improved alveolar structure (p<0.05) (FIG. 31B) when compared to untreated control. Treatment with CMC2.24 significantly reduced total call number in the SALT of emphysematous SP-D KO mice. Total cell numbers in the BAL fluid of CMC 2.24-treated mice and control mice (vehicle treatment) were determined by a hemocytometer method. The results indicate that CMC 2.24 treatment significantly reduced total cell number (p<0.05) in the lung of emphysematous mice when compared to control (vehicle-treated mice) (FIG. 32).

Treatment with CMC 2.24 changed phenotype of alveolar macrophages from non-health to health status in emphysematous SP-D MO mice: Typically, alveolar macrophages in emphysematous SP-D KO mice are ballooned with foamy, vacuolated cytoplasm (FIG. 3A). In the treatment with CMC 2.24 the phenotype of alveolar macrophages become healthy and normal phenotype of alveolar macrophages (FIG. 33B). Furthermore, the number of alveolar macrophages in the treated mice decreased (p<0.05) when compared to control (untreated mice).

Treatment with CMC 2.24 significantly reduced MMP-2 and -9 activity in the BALF of CMC 2.24-treated mice: Elevated levels of matrix metalloproteinases (MMPs) 2 and 9 are closely associated with lung parenchymal destruction in the progressive emphysema. So the levels of MMPs 2 and 9 activities in the BALF were determined using gelatin zymography. The data show the levels of MMPs 2 and 9 activities were significantly reduced in the CMC 2.24-treated mice (p<0.05) when compared to control (untreated mice) (FIG. 34).

### Example 16. Pulmonary Pneumonia

Mice: hTG SP-B mice carrying either human SP-B C or T allele without mouse SP-B gene background were generated and used. The SP-B mice were bred at least 10 generations to stabilize the transgenic SP-B expression. The genotypes of humanized SP-B-T/C mice were confirmed by PCR analysis. Mice were divided into three groups: the pneumonia group (Pneu, S. aureus infection only), pneumonia plus CMC2.24 treatment group (Pneu+CMC2.24, S. aureus infection plus CMC2.24), control group (sham, treated with sterile vehicle). All protocols related to animal experiments were approved by the institutional animal care and use committee of SUNY Upstate Medical University. Experiments were performed according to the National Institutes of Health guidelines and ARRIVE guidelines on the use of laboratory animals.

Curcumin derivative: Chemically-modified curcumin (CMC2.24) was dissolved in 1 ml suspension of 2% carboxymethyl cellulose vehicle for the daily oral administration (Jobin, C. et al. 1999; Wang, X. et al. 2012; Balasubramanyam, M. et al. 2003). The vehicle alone was administered in the control group.

S. aureus—induced pneumonia model: Pilot experiments were performed to establish the S. aureus Xen36 pneumonia model using different doses of bacteria to infect mouse lung. The results indicated a dose of 5×10⁸ CFU/mouse in 50 μl of bacterial solution was appropriate, because mice infected with this dose of bacteria could produce enough bioluminescent signal in the lung to be detected by the in vivo imaging system, consequently the infected mice had a reasonable survival rate at 48 h after infection. Therefore, direct intratracheal inoculation of bioluminescent S. aureus Xen36 at a dose of 5×10⁸/50 μl was used to infect mice in all subsequent experiment (Farnsworth, C. W. et al. 2015; Schriever, M. P. et al. 2011). In brief, hTG SP-B mice between 8 and 12 weeks old were anesthetized using intraperitoneal ketamine/xylazine (90 mg/kg ketamine, 10 mg/kg xylazine) injection. A 0.3-cm mid-line neck incision was made to expose the trachea. In the sham group, 50 μl of sterile vehicle was injected into the trachea by the same method. After infection, bio-luminescence signal was observed and quantified by an in vivo imaging system (Xenogen-200 series, Caliper Life Sciences, Hopkinton, MA). Buprenorphine (0.05 mg/kg body weight) was injected for postoperative analgesia every 8-12 hrs. Mice were returned to their cages in a temperature-controlled room (22° C.) with 12-h light and dark cycles and monitored every 4 h. Mice were anesthetized with isoflurane (2%) at several time points after infection (Oh, 12 h, 24 h, 28 h, 32 h, and 48 h) (Pribaz, J. R. et al. 2012; Guo, Y. et al. 2013). At 48 h after S. aureus infection, mice were sacrificed under anesthesia. Blood and bronchoalveolar lavage fluid (BALF) were collected for further study.

In vivo imaging analysis: The mice were observed for 46 hours after infection. Photographs were captured with a cooled CCD camera (Xenogen-200 series, Caliper Life Sciences, Hopkinton, MA). Pseudo-colored images of photon emissions were covered on gray scale images of the mouse to obtain spatial localization of the bioluminescent signals. For in vivo imaging: 5 mice were placed in the induction chamber at one time and anesthetized with isoflurane (2% in oxygen), and then placed into the IVIS-200 imaging chamber with continuous anesthesia. Images were performed for an initial exposure time of 5 min by in vivo imaging system (Rowe, J. et al. 2010).

Inflammatory call analysis in BAL: After harvest of BALF, the BALF was centrifuged by 250×g. The supernatants were saved in −20° C. freezer for further analysis. The pellets were re-suspended and wash with 1 ml of sterile saline, and then the cells were mounted on the slide by cytospin centrifuge at 1000 rpm for 3 min. Slides were stained with using the Hema-3 Stain Kit. Cells were examined by Nikon Eclipse TE2000-U research light microscopy (Nikon, Melville NY).

Histopathological analysis: After sacrifice, the lungs were fixed in 10% neutral formalin for at least 24 hours, and embedded in paraffin. Approximately 5 μm-slides of lung tissues from eight mice for each group were prepared and stained with Hematoxylin and eosin (H&E). Digital photos were taken with a light microscope (Nikon, Melville NY) and used for quantitative analysis according to the histological lung injury score system as described previously (Matute-Bello, G. et al. 2011). In brief, lung slides were evaluated using a 0-2 scale by two experienced investigators. The presence of alveolar (A) and interstitial neutrophils (B), alveolar hyaline membranes (C), proteinaceous debris (D) filling the air-spaces, and alveolar septal thickening (E) were scored in twenty high power fields for each slide. The resulting scores were calculated by the following formula: Score=[(20×A)+(14×B)+(7×C)+(7×D)+(2×E)]/(number of fields×100).

Apoptotic cells by TUNEL assay: About 5 μm-sections were incubated at 60° C. for 20 min, and then de-paraffinized in xylene twice every 10 mins, treated with different concentration-grades of alcohol [100%, 90%, 80% and 70% ethanol/ddH₂O], and then rinsed in phosphate buffer saline (PBS, pH7.5). Apoptotic cells were staining with deoxynucleotidyl transferase-mediated dUTP nick-end labeling (TUNEL) kit (Roche, Indianapolis, IN) by following the manufacturer's instruction (Liu, J. et al. 2015). Cell apoptosis was quantified by numbers of TUNEL-positive cells in 20 random fields at ×400 magnification (Liu, J. et al. 2015)).

Western blotting analysis: Frozen lungs were dissolved and homogenized with RIPA buffer with cocktail of protease inhibitors and phosphatase inhibitors (Roche), and the supernatants were used for Western blot analysis (Liu, J. et al. 2015)). The total protein concentrations of samples (lungs and BALF) were determined using the BCA micro assay kit (Thermo). Total protein (40 μg) was resolved by reducing (for NF-κB, Caspase-3, Bcl-2, p38/phosphorylated p38) and non-reducing (for SP-B) 12% SOS-polyacrylamide gel electrophoresis, and then transferred onto PVDF membranes at 4° C. (Bio-Rad, USA). After, the blot was blocked in 5% non-fat milk of Tris-buffered saline, detected using a primary anti-mouse/rabbit antibody against NF-κB (1:400, Santa Cruz Biotechnique), Caspase-3 (1:400, Santa Cruz Biotechnique), and Bcl-2 (1:400, Santa Cruz Biotechnique), as well as an anti-rabbit SP-B antibody (1:2000), and then an anti-rabbit/mouse secondary antibody conjugated with horseradish peroxidase was applied (Liu, J. et al. 2015)). β-actin anti-body (1:400, Santa Cruz Biotechnique) were used to strip and re-probe the membrane. Immuno-products were detected using Pierce ECL Western Blotting Substrate (Thermo Scientific) and the blots were exposed to X-film (Pierce Biochemicals, FL). Human BALF and proteins from sham mouse lung tissue were used as controls. The bands on films were quantified by Image J software version 1.48 (Wayne Rasband, NIH, Bethesda, MA).

MMPs activity by zymography: Total proteins (20 μg) from supernatants of BALF were loaded onto a 10% polyacrylamide gel containing 0.1% (wt/vol) gelatin under non-reducing conditions to examine MMP-2 and MMP-9. After electrophoresis, the gel was washed with renaturing buffer (2.5% Triton X-100) for 30 min, incubating with 100 mL of developing buffer (40 mM Tris, 200 mM NaCl, and 10 mM CaCl2); pH 7.5) at room temperature for 30 minutes and then at 37° C. for 24 h with gentle agitation. The gel was then stained in 0.05% (wt/vol) Coomassie Brilliant Blue, 30% (vol/vol) methanol, and de-stained in 10% (vol/vol) acetic acid for 1 h and repeatedly for additional 3 h. For MMP-12 expression, BAL fluids (20 μg of protein) were used on a 12% polyacrylamide gel containing 0.05% (wt/vol) casein following the same protocol. Densitometry was carried out using Image J software version 1.48 (Wayne Rasband, National Institutes of Health, Bethesda, MA).

Statistical analysis: All data are presented as means±SEM. Data were compared using Student t-test or ANOVA by Sigma Stat software (version 3.5). Animal survival analysis was performed by a Kaplan-Meier survival method. For all comparisons, p<0.05 was considered statistically significant.

### Results

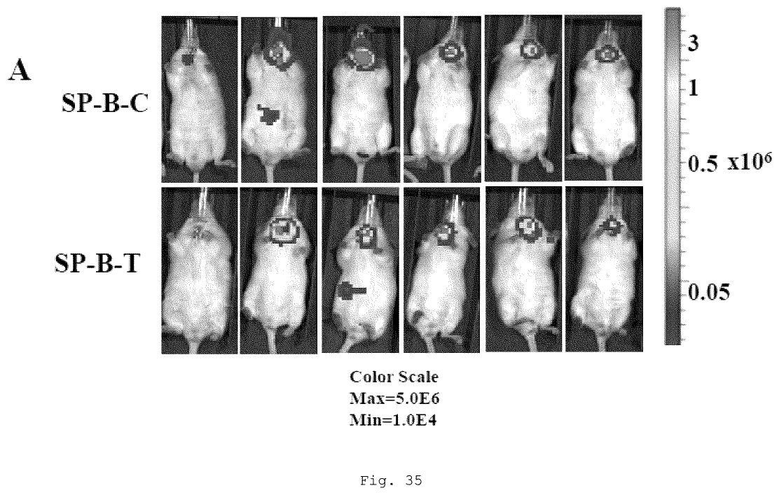

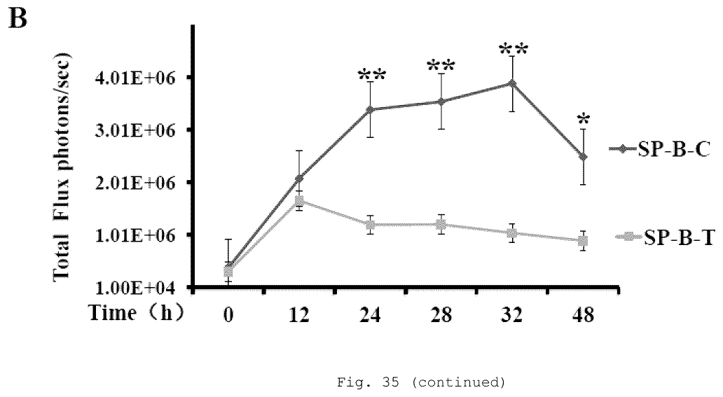

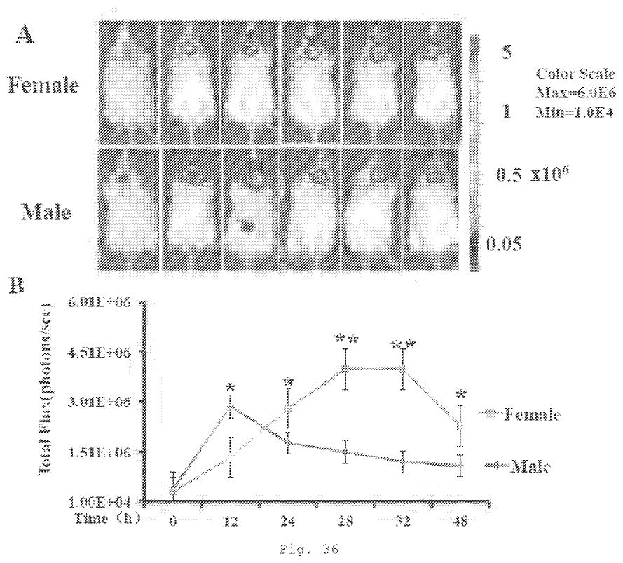

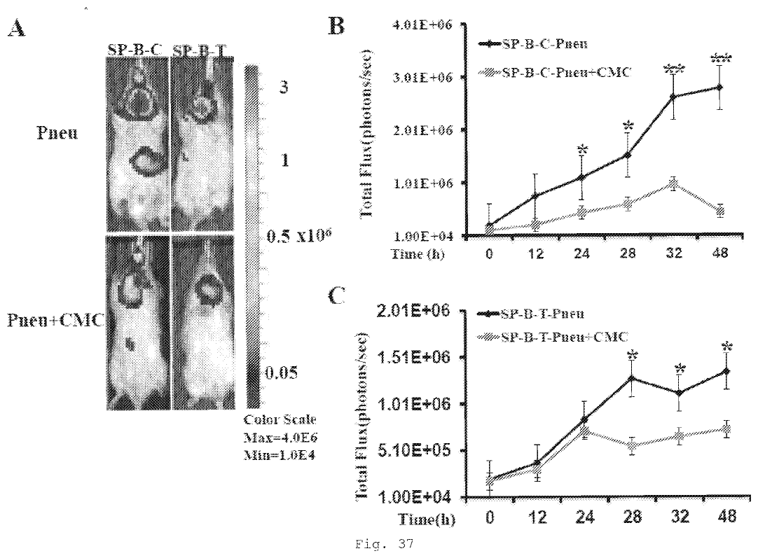

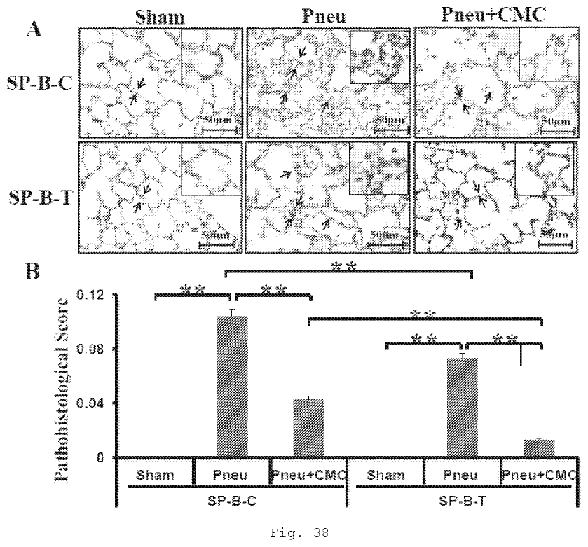

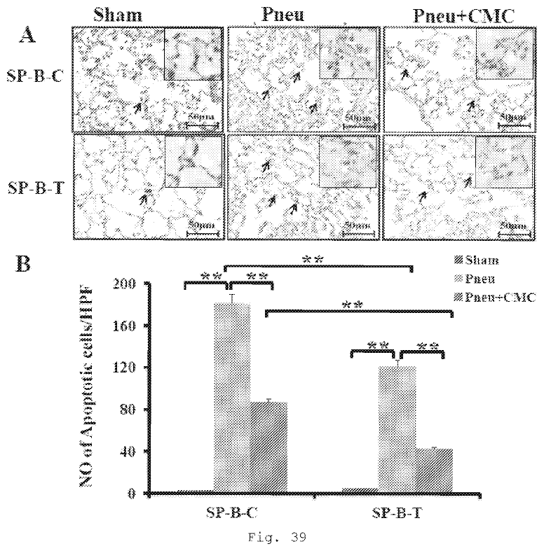

Zn vivo measurement to S. aureus infection in hTG SP-B-C and SP-B-T mice using bioluminescence analysis: To study functional differences of human SP-B genetic variants in the bacterial pneumonia bacterial dynamic changes in the lungs of hTG SP-B-C and SP-B-T mice after intratracheal infection of bioluminescent labeled S. aureus at six time points i.e. 0, 12, 24, 28, 32, 48 hours after infection were measured. The results from in vivo image analysis showed the level of bioluminescence was significant higher (p<0.01) in the infected SP-B-C mice from 24 h to 48 h after infection compared to infected SP-B-T mice (FIG. 35). For infected SP-B-C mice, the levels of bioluminescence increased rapidly from 0 to 24 h after infection, and the level kept high from 24 to 32 h, then decreased (FIG. 35B). In infected SP-B-T mice, the peak of bioluminescence was 12 h after infection, and then the level decreased slowly (FIG. 35B). In addition, there is different mortality rates between infected SP-B-C and SP-B-T mice (62.8% vs. 33.3%, p<0.01) by 48 h after infection, respectively. These results indicate greater resistance to S. aureus Xen36 bacterial infection exists in SP-B-T mice compared to SP-B-T mice.

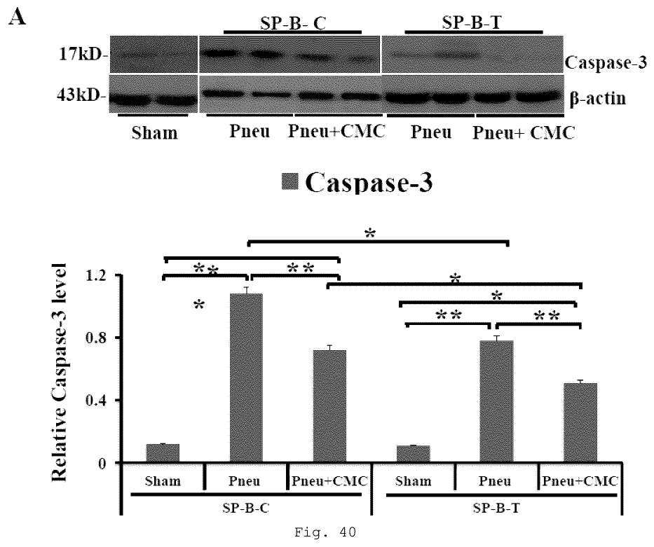

The effect of gender on S. aureus infection using bioluminescence analysis: The effects of male and female gender on bacterial infection using in vivo bioluminescence imaging were examined. The results demonstrate significant differences in bacterial dynamic changes in the lung of infected male and female mice at several time points i.e. 12, 24, 28, 32, and 48 hours (FIG. 36). In male mice, the level of bio-luminescence in the male mice peaked 12 h after infection, then decreased gradually until 48 hours. At 12 h after infection, the level of bioluminescence in the male mice was higher (p<0.05) than that in the female mice, but at 24, 28, 32 and 48 h, infected female mice had higher level of bioluminescence compared to infected male mice (FIG. 36B). Additionally, female mice had a significantly higher different mortality than male mice (36.8% vs. 48.15%, p<0.05).

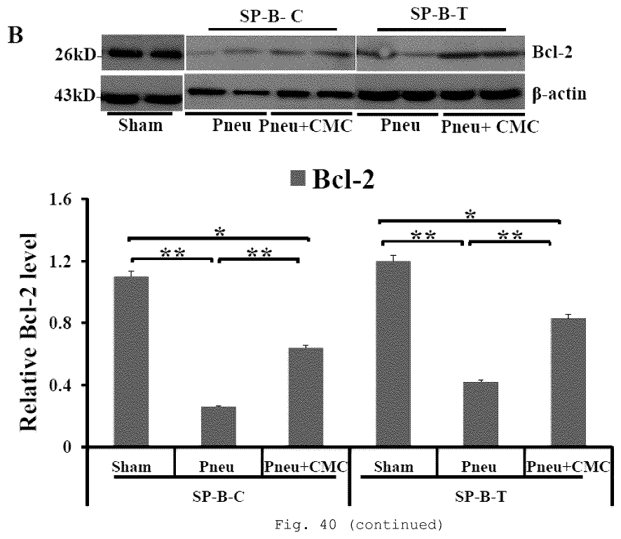

The effect of CMC2.24 on S. aureus resistance in hTG SP-B-C and SP-B-T mice using in vivo bioluminescence analysis: To study the effect of CMC2.24 in bacterial pneumonia, infected SP-B-C and SP-B-T mice were administered a daily dose of CMC2.24 (50 mg/kg) or vehicle (control). The results showed significantly decreased bacterial load in the CMC2.24 treated group compared to the control (FIG. 37). For SP-B-C mice, the levels of bio-luminescence were significantly lower (p<0.01) in the CMC2.24 treated group from 24 h to 48 h after infection, compared to the control (FIG. 37B). Similar effects were observed in SP-B-T mice (FIG. 37C). Furthermore, we observed decreased mortality rate in the CMC2.24 treated SP-B-C mice compared to the SP-B-C control (50% vs. 76%, p<0.05), though this was not observed in the SP-B-T mice (32% vs. 33%).

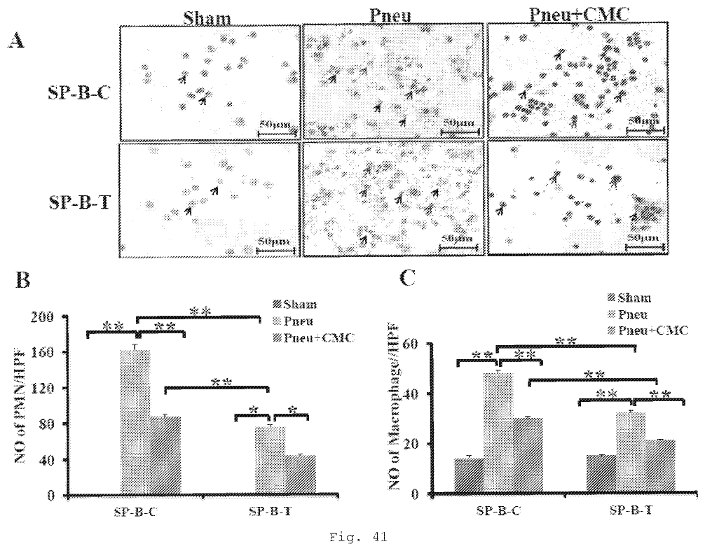

Lung histology: To assess the effects of human SP-B genetic variants and CMC2.24 on lung injury in the pneumonia we examined lung histopathology of three groups (Sham, Pneu, Pneu+CMC) at 48 h after infection. The results showed obvious changes in lung injury 48 h after infection with or without CMC2.24 treatment (Pneu, Pneu+CMC) but not in Sham mice (FIG. 38A). CMC2.24 treated mice showed decreased lung injury by histology and scores compared with control mice 48 h after infection, including fewer neutrophils in the alveolar space and interstitial membrane, decreased accumulation of proteinaceous debris, and thinner alveolar walls in the lung (FIG. 38A). Furthermore, quantitative analysis indicates the lung injury scores of both CMC2.24 treated SP-B-C and SP-B-T mice (Pneu+CMC) are lower (p<0.01) compared to the control mice (Pneu), but larger than that of Sham mice (FIG. 38B). In addition, the lung injury scores of infected SP-B-C mice with and without CMC2.24 are larger (p<0.01) than those of infected SP-B-T mice with and without CMC2.24, respectively (FIG. 38B).

Lung apoptosis: First, apoptotic cells and apoptosis-related protein (biomarker) expression in the lung tissues of three experimental groups were examined i.e. pneumonia (Pneu), or pneumonia plus CMC2.24, as well as Sham mice by TUNEL assay. As shown in the FIG. 39A, apoptotic cells exhibit brown nucleus in infected mice but not for Sham mice. Lung tissues from of infected SP-B-C mice (Pneu) showed more apoptotic cells compared to infected SP-B-T mice (Pneu) (p<0.01) (FIGS. 39A and 39B). CMC2.24 treated mice showed decreased apoptotic cells (p<0.01) when compared to their respective controls (Pneu).

Expression of two apoptosis-related proteins was also examined in the lung tissues by Western blot analysis. Caspase-3 (Cap-3), as one biomarker of one ongoing cell apoptosis, has correlated positively with apoptosis. The results showed significant increase of Cap-3 expression in the lungs of infected SP-B-C and SP-B-T mice compared to Sham mice (FIG. 40A, p<0.01). CMC2.24 treated SP-B-C and SP-B-T mice showed decreased levels of Cap-3 expression compared to their respective control mice (FIG. 40A, p<0.01). In addition, another biomarker of apoptosis was examined, Bcl-2 as an inhibitor of apoptosis. The expression of Bcl-2 decreased in infected SP-B-C and SP-B-T mice compared to Sham mice (FIG. 40B, p<0.01). CMC2.24 treatment caused increased levels of Bcl-2 expression in the lung tissues from infected SP-B-C and SP-B-T mice compared to SP-B-C(p<0.01) and SP-B-T (p<0.05) control mice, respectively (FIG. 40B).

Inflamatory cells in BALF: Inflammatory cells in the BALF from the different experimental groups: Pneu, Pneu+CMC, as well as Sham mice were assessed. As shown in the FIG. 41, the BALF from Sham mice had more than 98% of alveolar macrophages without neutrophils. A larger amount of inflammatory cells (neutrophils and macrophages/monocytes) were observed in the BALF of Pneu mice, along with decreased neutrophils in the BALF of Pneu+CMC treated mice. Quantitative analysis showed the number of neutrophils in the BALF of infected SP-B-C and SP-B-T mice with or without CMC2.24 was larger than that of Sham mice (FIG. 41B, p<0.01). The number of neutrophils decreased significantly in the BALF of both SP-B-C(p<0.01) and SP-B-T (p<0.05) after CMC2.24 treatment compared with their respective controls (FIG. 41B). Similar results were observed for macrophages/monocytes in the BALF from infected SP-B-C and SP-B-T mice.

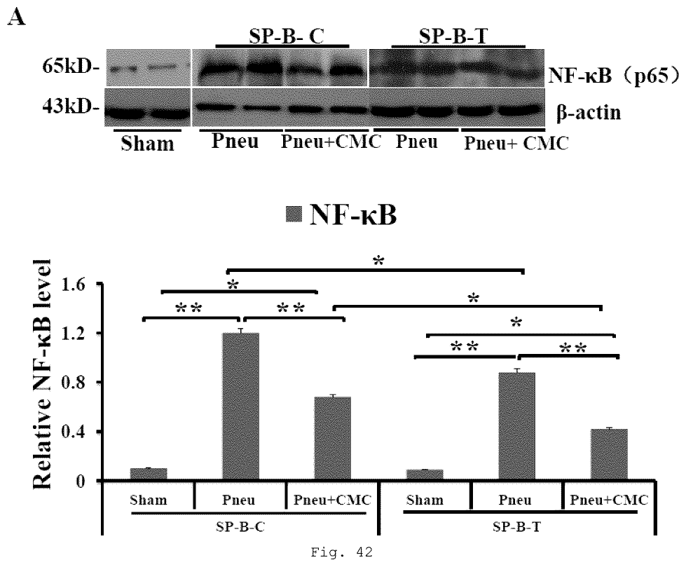

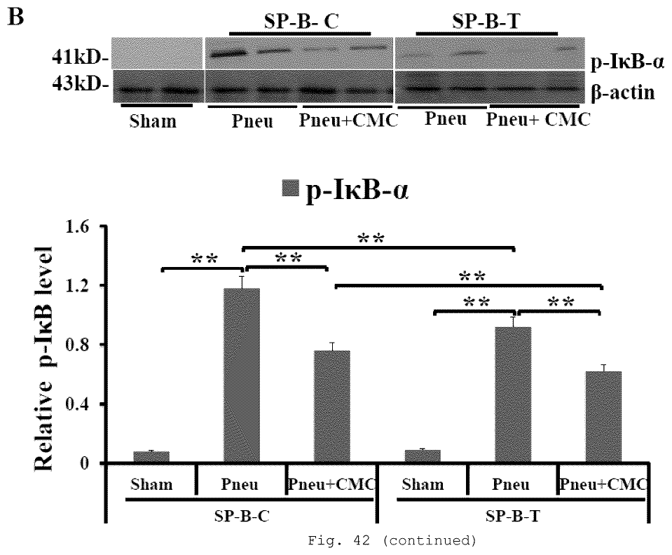

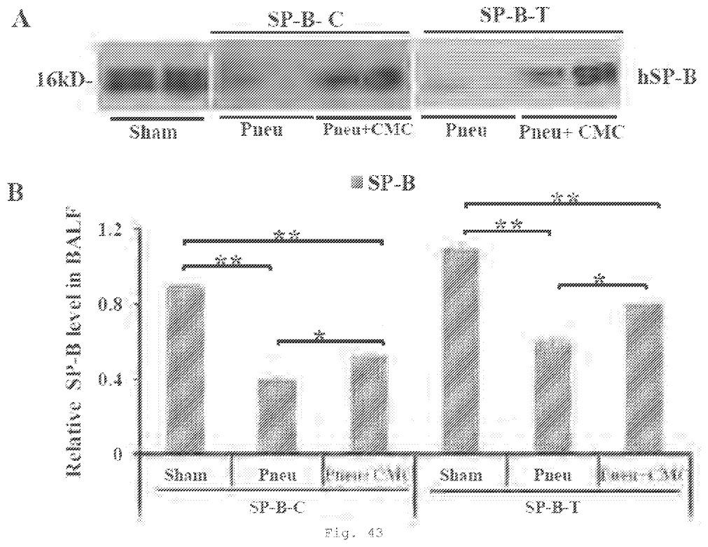

Lung NF-κB activation: Previous studies have shown that one of the SP-B gene products is involved in host defense (Yang, L. et al. 2010) and curcumins can regulate host inflammation induced by sepsis through attenuating NF-κB activation (Jobin, J. et al. 1999; Xiao, X et al. 2012). Therefore the levels of NF-κB p65 and phosphorylated-IκB-α (p-IκB-α) in the lung using Western blotting analysis with antibodies against NF-κB p65 and p-IκB-α were examined. The results showed increased levels of NF-κB p65 and p-IκB-α in the lung of infected groups (pneu and Pneu+CMC) compared to Sham mice (FIG. 42, p<0.01). Differences of the levels of NF-κB p65 and p-IκB-α expression were determined in infected SP-B-C and SP-B-T mice (FIG. 42, p<0.05). The levels of NF-κB p65 and p-IκB-α in the lungs of infected SP-B-C mice were higher than those observed in CMC2.24 treated mice (FIG. 42, p<0.01). The levels of NF-κB of p-IκB-α in infected P—B-C were significantly higher than that of infected SP-B-T mice (p<0.05).

SP-3 levels in BALF: The levels of SP-B protein in the BALF were determined from hTG SP-B-C and SP-B-T mice at 48 h with pneumonia (Pneu), or pneumonia plus CMC2.24, as well as Sham mice. The level of SP-B protein in BAL fluids from sham mice were higher than those observed infected mice (FIG. 43A, p<0.01). SP-B levels in BALF from CMC2.24 treated SP-B-C and SP-B-T mice were higher than their respective controls (FIG. 43B, p<0.05).

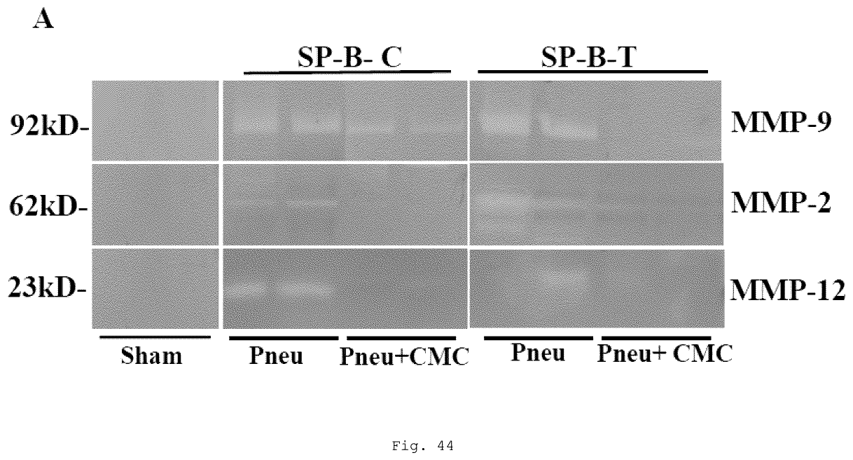

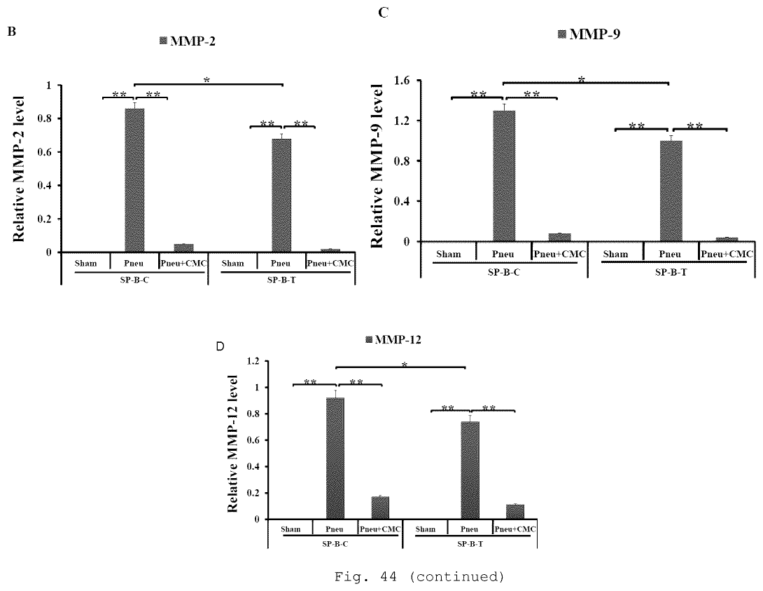

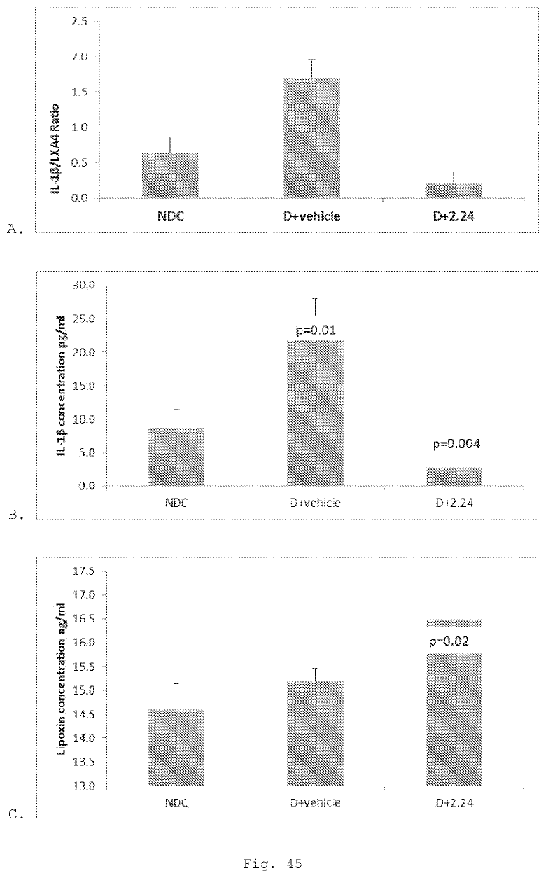

MMs activity in BALF: Previous studies have shown CMC2.24 can inhibit MMPactivitu (Zhang, Y. et al. 2012; Corbel, M. et al. 2000). Therefore, MMP-2, -9, and -12 activities were examined in the BALF using zymographic analysis. Our results demonstrate the BALF from sham mice has minimal MMPs activity of MMP-2, -9, and -12; but infected SP-B-C and SP-B-T mice demonstrate increased MMP-2, -9, and -12 activities (FIG. 44A, p<0.01). CMC2.24 treated SP-B-C and SP-B-T mice showed decreased levels of MMP-2, -9, and -12 activities compared to their respective controls (FIG. 44B-D, p<0.05).

### Example 17: S. aureus Pneumonia

Staphylococcus aureus is a common cause of nosocomial pneumonia frequently causing acute respiratory distress syndrome (ARDS). Surfactant protein B (SP-B) gene expresses two proteins involved in lowering surface tension and host defense. Genotyping studies demonstrate a significant association between human SP-B genetic variants and ARDS. Curcumins have been shown to attenuate host inflammation in many sepsis models. It was found that mice with SP-B-C allele are more susceptible to S. aureus pneumonia than mice with SP-B-T allele; and that CMC2.24 improves mortality and attenuates lung injury. Humanized transgenic mice, expressing either SP-B T or C allele without mouse SP-B gene, were used. Bioluminescent labeled S. aureus Xen36 (50 μl) was injected intratracheally to cause pneumonia. Infected mice received daily CMC2.24 (50 mg/kg) or vehicle alone (control) by gavage. Dynamic changes of bacteria were monitored using in vivo imaging system. Histological, cellular and molecular indices of lung injury were studied in infected mice 48 h after infection. In vivo imaging analysis revealed total flux (bacterial number) was higher in the lung of infected SP-B-C mice compared to infected SP-B-T mice (p<0.05); difference of bacterial dynamic growth exists between male and female mice. Infected SP-B-C mice demonstrated increased mortality, lung injury, apoptosis and NF-κB expression compared to infected SP-B-T mice. Compared to control, CMC2.24 treatment improved mortality, reduced total flux and apoptosis, decreased inflammatory cells, NF-κB expression (p<0.05), and less MMPs-2, -9, -12 activities (p<0.05).

A novel humanized transgenic mice which expresses either SP-B T or C allele without mouse SP-B gene has been established. It was found that hSP-B-C allele is more susceptible to S. aureus pneumonia than mice with SP-B-T allele. It was found that CMC2.24 improves mortality and attenuates lung injury.

### Example 18: Chronic Bronchitis and Goblet Call Metaplasia

Chronic obstructive pulmonary disease (COPD) is a progressive lung disorder including two underlying conditions: chronic bronchitis and emphysema. Chronic bronchitis causes inflammation/fibrosis of the small airways, airway obstruction with increased mucus secretion, and abnormal inflammatory response to external stimuli. COPD is the third-leading cause of death in the United States. PM_{2.5}, one of the most dangerous components of air pollution, causes a great health risk. Due to its small size (<2.5 μm), it can reach alveolar spaces of the lung and induce lung inflammation. CMC_{2.24}, a compound from chemically modified curcumin, has higher bioactivity and better solubility compared to natural curcumin products. PM_{2.5} exposure induces chronic bronchitis exacerbation and CMC2.24 can attenuate lung injury in chronic bronchitis mouse model and PM2.5-induced bronchitis exacerbation. Mice treated with elastase and LPS once a week for 4 weeks were subsequently administered 125 μg PM_{2.5} by intratracheal injection followed by (40 mg/kg) CMC2.24 or vehicle (control) by gavage for seven days. Mice behavior, Lung histology and inflammation were examined. Bronchoalveolar lavage (BAL) was analyzed using molecular and cellular methods. Elastase/LPS-exposed mice showed typical characteristics of chronic bronchitis in lung including: a) lung injury, b) widespread inflammatory changes, c) aggregations of neutrophils and mononuclear inflammatory cells in the perivascular and peribronchiolar spaces, d) goblet cell metaplasia. After exposure to PM_{2.5}, these changes were more pronounced with the significant increase in MMP activity. With CMC2.24 treatment, the mice showed improved muscle strength and overall activity, reduced chronic bronchitis and goblet cell metaplasia.

A novel PM_{2.5}-exposure induces bronchitis exacerbation model has been established. In addition, CMC2.24 can attenuate chronic bronchitis in the elastase/LPS mouse model and PM2.5-induced bronchitis exacerbation.

### Example 19. CMC2.24 Increases Lipoxin, Resolvin and Cytokine Levels in Subjects Afflicted with Pulmonary Bacterial Pneumonia

An amount of CMC2.24 is administered to a subject afflicted with pulmonary bacterial pneumonia. The amount of the compound is effective to treat the subject by inducing production of the one or more lipoxins in the subject. The amount of the compound is effective to treat the subject by inducing production of the one or more lipoxins in the subject and one or more resolvins. The amount of the compound is effective to treat the subject by inducing production of the one or more lipoxins in the subject, one or more resolvins and one or more anti-inflammatory cytokines in the subject.

### FIG. 20: In Vivo Studies

### Experiment A:

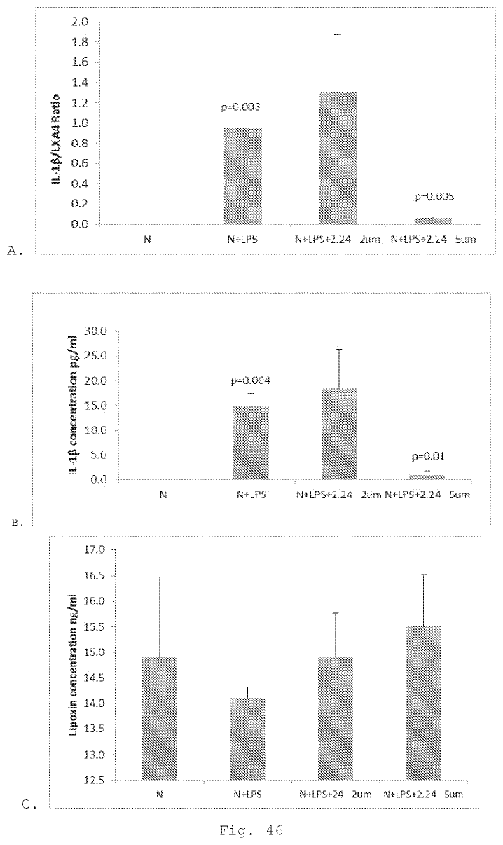

In an in vivo experiment, three groups of adult male rats were established including non-diabetic controls (NDC group; n=6 rats/group); rats that were made diabetic and severely hyperglycemic by STZ injection (D+vehicle group; n=6 rats/group), and a 3^{rd} group of rats (n=6 rats/group) in which the diabetics were orally administered CMC2.24 (30 mg/kg body weight) once per day for 21 days. At the end of the protocol, the rats were sacrificed and the peritoneal cells were collected by washing with cold 15 ml phosphate buffered saline/EDTA. The macrophages were harvested from the peritoneal wash from each rat, after 2 hours of adherence to culture plates (sterile conditions), the cells were counted and then incubated for 18 h at 37° C. in an atmosphere of 95% air/5% C02. The conditioned media was then collected and analyzed for the resolvin, lipoxin A4, and for two inflammatory cytokines, IL-1β and IL-6. The data is expressed as a ratio of IL-1β (pg/ml) relative to resolvin (ng/ml) secreted by the macrophages from the three experimental groups (FIG. 45A).

Inducing diabetes resulted in a 183% increase in the inflammatory mediator (IL-1β) relative to the resolving (lipoxin A4), a ratio indicating a hyper-inflammatory state due to this imbalance (note that the levels of the long-lived inflammatory cytokine, IL-6 were too low to be detected in this cell culture system). However, when the diabetic rats were orally administered CMC2.24, the macrophages from these treated rats (even though the severity of hyperglycemia was not reduced) showed a dramatic reduction of 85.3% compared to the untreated diabetics (FIG. 45A). These data demonstrate the potent ability of CMC2.24 to sharply reduce the severity of the hyper-inflammatory state in a severely diabetic mammal. This hyper-inflammatory state in the diabetic rats, which were NOT treated with CMC2.24, is due to a dramatic increase in the concentration (pg/ml) of the inflammatory cytokine, IL-1β, with little or no increase in the resolving, lipoxin A4 (FIG. 45B & 45C). In contrast, when the diabetic rats were orally administered CMC2.24, the resolving secretion by the macrophages was significantly increased (p=0.02), and the inflammatory cytokine (IL-1β) was dramatically decreased (p=0.004) which corrected this hyper-inflammatory condition even though the severity of hyperglycemia was unaffected by the CMC.

### Experiment B:

In the second experiment, macrophages (chronic inflammatory cells), were collected and counted from the peritoneal washes of the six non-diabetic control rats. These cells were then pooled and incubated under different in vitro conditions including: group 1, control Møs; group 2, Møs incubated with lipopolysaccharide (LPS)/endotoxin at 100 ng/ml, final concentration, added to the culture media; group 3, Møs exposed to LPS but treated with CMC2.24 at a final concentration of 2 μM; and group 4, like group 3 except that CMC2.24 was increased to 5 μM (note that in this cell culture experiment, sufficient IL-6 was secreted by the LPS-exposed Møs unlike Experiment A, above).

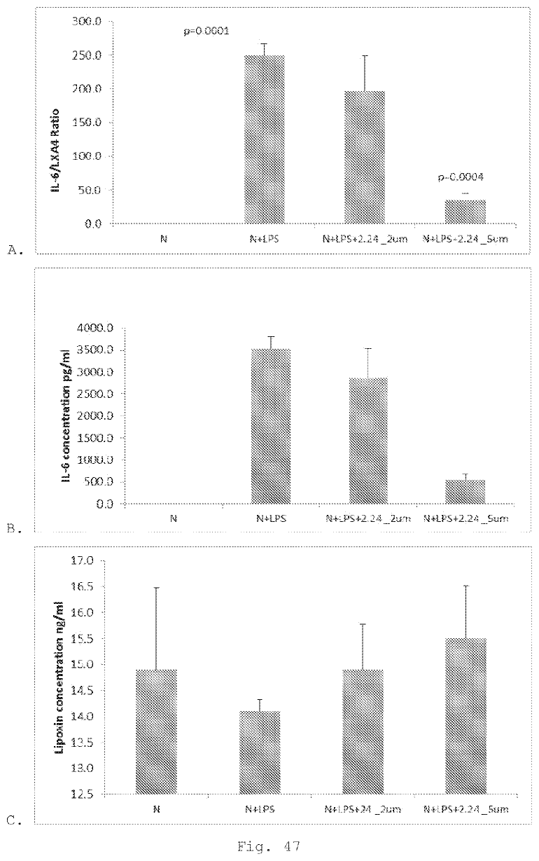

Of interest, the proportion of the two inflammatory cytokines, IL-1β and IL-6, relative to the resolvin/anti-inflammatory mediator, lipoxin A4, responded in a similar fashion—that is, little or no IL-1β and IL-6 were produced by the Møs NOT exposed to the bacterial endotoxin, LPS, in cell culture. In contrast, exposing these chronic inflammatory cells to the bacterial LPS significantly increased the hyper-inflammatory ratio (p=0.003 for IL-1β, and p=0.0001 for TL-6) relative to the resolvin, lipoxin A4 (FIGS. 46 & 47). Treating the LPS-exposed Mms in culture to 2 μM CMC2.24 did not significantly reduce this hyper-inflammatory ratio. However, increasing the concentration of CMC2.24 to 5 μM did produce significant resolution of these inflammatory mediators, i.e., CMC2.24 reduced the IL-1β ratio by 93.8% (p=0.005; FIG. 46A) and reduced the IL-6 ratio by 86% (p=0.0004; FIG. 47A). For additional details see FIGS. 46B and 46C, and FIGS. 47B and 47C, to see the changes in lipoxin A4, IL-1β, and IL-6 concentrations.

### DISCUSSION

Curcumin has shown promise as a platform for the development of drugs to target many diseases and syndromes, including cancer and inflammatory diseases, as well as anthrax; however, one of the major obstacles to overcome in considering curcumin for further drug development has been its relatively low bioavailability (Mock, M. et al. 2001). Despite this, studies by Zhang et al. show that curcumin and CMC2.24 bind fairly strongly to bovine serum albumin (Zhang, Y.; Golub L. M. et al. 2012), and when considering normal plasma concentrations of serum albumin, this should provide sufficient capacity to carry high enough concentrations of curcumin or CMC2.24 through the blood, increasing the half-time of their decomposition from mere minutes to tens of hours or days. In this same study, curcumin and CMC2.24 administered by oral gavage to rats expressing pathologically excessive levels of MMPs showed no evidence of toxicity, even in doses as high as 500 mg/kg of body weight (Zhang, Y. et al. 2012). Through chemical modification, it has now proven possible to synthesize derivatives of curcumin that have improved solubility, stability, and potential bioavailability, while still retaining or improving upon the inhibitory potency and negligible toxicity of the parent compound. Some of these CMCs have been found to have inhibitory potencies greater than or equal to curcumin itself against several of the matrix metalloproteinases.

One of these CMCs in particular, CMC2.24, has shown exceptional promise in other systems, and is thus given prominence in this and other papers. CMC2.24 shows improved solubility and even less toxicity in cell and tissue culture, as well as in in vivo studies, when compared to the parent compound (Zhang, Y. et al. 2012). The modifications to curcumin in synthesizing CMC2.24 include subtraction of the methoxy groups from the 3′ positions of curcumin's flanking aromatic rings, as well as the addition of a phenyl group, which is connected to the center of the molecule via a peptide bond. This modification provides CMC2.24 with an additional carbonyl capable of participating in keto-enol tautomerization, as well as several additional resonance structures, and a third hydrophobic region at its periphery. Studies by Zhang et al. show that CMC2.24 is nearly 10-fold more acidic than curcumin itself (Zhang, Y.; Golub L. M. et al. 2012), and exists largely as an enolate rather than an enol at physiological pH, which is likely a consequence of the additional electron-withdrawing group. This difference also seems responsible for CMC2.24's greater solubility, and superior zinc-binding ability (Zhang, Y.; Golub L. M. et al. 2012).

Chronic (and systemic) inflammation is associated with poorly controlled diabetes. Functions of chronic inflammatory cells, notably macrophages, can be impaired contributing to diabetic complications. The effect of CMC2.24 on macrophages in an animal model of severe type I Diabetes (in vivo) and in cell culture (in vitro) was evaluated. It was found that this compound not only reduced the excessive accumulation of macrophages in peritoneal exudates in vivo, but also normalized impaired cell function without affecting the severity of diabetes assessed by blood glucose levels. This compound is effective in treating chronic inflammatory diseases other than diabetes (e.g., rheumatoid arthritis) not by inhibiting the inflammatory response, like NSAIDs and corticosteroids, but by improving the “competence” of inflammatory cells (e.g., macrophages) and increasing production of lipoxins, resolvins and/or cytokines, thus reducing the abnormal and tissue-destructive prolongation of chronic inflammation, i.e., our new compounds “resolve” but don't “suppress” the acute inflammatory response, thus preventing it from becoming chronic.

CMC2.24, synthesized as reported previously (Zhang, Y. et al. 2012), was examined for its ability to induce lipoxin production in diabetic rats (FIG. 1). Based on the dynamics of the inflammatory response with time, and its impairment by severe hyperglycemia and “normalization” by this novel compound, it is concluded that CMC2.24 is useful in resolving inflammation by increasing production of lipoxin A4, an anti-inflammatory lipoxin.

### COPD

Chronic Obstructive Pulmonary Disease (COPD) is a progressive disorder of the lung parenchyma characterized by chronic inflammation, increased mucus secretion plugging small airways, emphysema, and an abnormal inflammatory response to external stimuli. This leads to partially reversible chronic-progressive airflow limitation due to chronic bronchitis, emphysema or both and also, in part, due to a loss of lung elasticity caused by enzymatic degradation of the lung matrix by proteases.

The exact cellular and molecular mechanisms of COPD pathogenesis is unknown, the main factors in development and progression of the disease are thought to be chronic inflammation, oxidative stress, and an imbalance of proteases and anti-proteases (Marumo, S. et al. 2014). Smoking and exposure to noxious airborne particles are the most important risk factors for triggering inflammation in patients with COPD Other factors found primarily in the developing world include exposures to dusts, fumes, air pollution particles, and biomass fuels (Churg, A. M. at al. 2008; Min, T. et al. 2011; Kurhanewicz, N. et al. 2014).

Although there are many new theories explaining alveolar wall destruction in COPD, the protease-antiprotease hypothesis remains the main theory for explaining the destruction of alveolar matrix that leads to emphysema. This hypothesis was formulated from the observation that humans deficient in α1-antitrypsin (A1AT) developed early emphysema and from animal experiments which showed that instillation of elastolytic enzymes produced emphysema in experimental animals (Churg, A. M. et al. 2008). Recent experiments demonstrate that COPD-like features can be induced in mice by exposure to a combination of LPS and elastase once a week for 4 weeks (Ganesan, S. et al. 2010).

Matrix metalloproteinases (MMPs) are proteolytic enzymes that are generally capable of degrading all components of the extracellular matrix (ECM) and basement membrane both in normal physiological and in abnormal pathological processes. MMPs are classified according to several criteria important among which is substrate specificity (Visse, R. and Nagase, H. 2003). One specific MMP, macrophage metalloproteinase (MMP-12), is produced mainly by macrophages and has the ability to degrade different substrates including elastin, the major component of alveolar walls. It is believed that MMP-12 plays an important role in the pathogenesis of COPD (Le Quement, C. et al. 2008). Another important MMP, Matrix metalloproteinase 9 (MMP-9), also known as gelatinase B, has a variety of substrates and diverse functions as modulation of inflammation, tissue repair and tissue remodeling. It has a multitude of substrates including gelatin, type IV and V collagens (Bratcher, P. E., et al. et al. 2012).

Particulate matter (PM) is a diversified mixture of gases, liquid and solid particles of different origins and sizes suspended in the air and are classified by size: coarse (2.5 to 10 μm diameter), Fine (0.1 to 2.5 μm diameter), and ultrafine (<0.1 μm diameter) (Riva, D. R. et al. 2011).

PM air pollution is widespread in the urban environment. PM_{2.5}-which is made from a mixture of metals, organic compounds, and other substances produced primarily from the combustion of petroleum products—is the most dangerous component of air pollution and poses the greatest health risk. Due to its small size, it passes all the way to the deepest reaches of the lungs and induces local and systemic inflammation. It is well established that a few hours to days of exposure to high levels of PM_{2.5} causes exacerbations of pre-existing lung conditions and results in excess emergency department Visits and hospitalizations for those with asthma, COPD, and pneumonia (Ostro, B. et al. 2007; Bernstein, A. S. et al. 2005; Kappos, A. D. et al. 2004; Ling, S. H. et al. 2009).

Exposure to PM either by inhalation or instillation induces inflammatory responses in humans and animals. Alveolar macrophages produce a broad range of cytokines, particularly IL-6, IL-8, and macrophage inflammatory protein (MIP-1), which leads to increased oxidative stress and vascular permeability coupled with neutrophil recruitment through the release of granulocyte macrophage colony-stimulating factor (GM-CSF). It also promotes increased expression of genes related to NF-κB activation, including TNF-α, TGF-β and IL-6 (Riva, D. R. et al. 2011; Ostro, B. et al. 2007; Bernstein, A. S. et al. 2005; Kappos, A. D. et al. 2004; Ling, S. H. et al. 2009). Pulmonary surfactant, a lipid and protein complex, is essential for respiratory physiological function because it prevents lung collapse by lowering alveolar surface tension. Surfactant-associated proteins consist of four functional proteins: surfactant protein A (SP-A), B (SP-B), C(SP—C), and D (SP-D). SP-A and SP-D are members of the C-type lectin (collectin) protein family and they plays an important role in host defense and regulation of inflammatory processes in the lung, where they are expressed and secreted by alveolar type II pneumocytes and bronchiolar Clara cells (Wright, J. R. et al. 2005; Crouch, E. et al. 2001; Wittebole, X. et al. 2010). SP-A and SP-D are hydrophilic proteins and participates in the function of surfactant activity (22). SP-A and SP-D also opsonizes pathogens, and enhances pathogen uptake by macrophages (Wittebole, X. et al. 2010), as well as binding to rough LPS present on the surface of gram-negative bacteria, inhibiting the growth of these bacteria by increasing membrane permeability (Poulain, F. R. et al. 1999; Wu, H. et al. 2003).

More importantly, SP-A and SP-D can modulate inflammatory processes through regulation of NF-κB activity such as blocking lipopolysaccharide (LPS) binding to the TLR4 receptor and CD14 receptor (Malloy, J. et al., 1997; Yamazoe, M. et al. 2008).

Curcumin has been used in the treatment of several inflammatory diseases including arthritis, digestive and liver abnormalities, and respiratory infections (Avasarala, S. et al. 2013). Studies showed that curcumin inhibit NF-kB activation, IL-8 release and neutrophil recruitment in the lungs. It acts as superoxide radical and hydroxyl radical scavenger, increases levels of glutathione by induction of glutathione cysteine ligase (GCL) (Shishodia, S., et al. 2013; Rahman, I. 2006).

A series of novel chemically modified curcumins (CMCs) were developed by Zhang et al. (33). Compared to natural curcumin these compounds have improved zinc binding and better bioavailability and a “lead” compound has been identified. This “lead” compound, CMC2.24, is a phenylamino-carbonyl curcumin. In contrast with the diketonic active site on the natural curcumin compounds, CMC 2.24 has a triketonic active site enabling enhanced zinc-binding. It has shown evidence of efficacy in vitro in cell and organ culture, as well as in vivo in animal models of chronic inflammatory diseases (Botchkina, G. I. et al. 2013; Zhang, Y. et al. 2012; Elburki, M. S. et al. 2014). CMC2.24 exhibits pleictropic anti-inflammatory effects and functions by inhibiting a broad-spectrum of inducible matrix metalloproteinases (iMMPs). CMC2.24 inhibits iMMPs in two ways: it directly inhibits multiple forms of iMMPs and it blocks the conversion from proenzyme to active enzyme. In addition, CMC2.24 inhibits production of pro-inflammatory cytokines such as IL-10, TNF-α, and IL-6, probably by interrupting the NF-kB pathway (Elburki, M. S. et al. 2014).

The current treatment regimens depend mainly on combinations of several medications with different therapeutic targets and include corticosteroids, 02-adrenoceptor agonists, leukotriene receptor antagonists, theophylline, and others. These therapies can produce potential side effects, including but not limited to growth retardation, the induction of insulin resistance, the loss of bone mass, immune suppression, gastrointestinal disturbances, and arrhythmias, and they do not consistently ameliorate airway inflammation in some COPD patients. In this examples, it was shown that nasal administration of Elastase/LPS weekly for four weeks induce COPD like features in the treated mice including widening of the alveolar spaces peribronchiolar and perialveolar infiltration with inflammatory cells and hyperplasia of goblet cells. It was also shown that challenging Elastase/LPS treated mice with intratracheal PM_{2.5} lead to exacerbation of COPD as evidenced by increase in lung histopathological index, increase in mean linear intercept, inflammatory cells in BAL and increased MMPs 2, 9 and 12 activities. It was further shown that concurrent treatment with CMC₂₋₂₄ prevented such exacerbation and attenuated the emphysematous and inflammatory conditions in the treated mice as evidenced by histological, cytological and histochemical examination.

The present findings with regard to the histological sections of the elastase/LPS-treated mice, such as alveolar space widening, and small airway inflammatory changes are in concordance with the findings of other investigators using the same protocol (Le Quement, C. et al. 2008; Elkington, P. T. et al. 2006; Halbert. R. J. et al. 2006), thus making it a useful COPD-mouse model. This model represents many features of the human disease and has advantages in comparison to the cigarette smoke model because the latter takes at least six months to develop and shows only mild to moderate emphysematous changes (Churg, A. et al. 2008; Wright, J. L. and Churg, A. 2008), and it lacks the features of chronic bronchitis and goblet cell metaplasia (Ganesan, S. et al. 2010). Exposure of COPD-mice to PM_{2.5} showed exacerbation of the inflammatory changes in the lung with greater neutrophil infiltration and the appearance of a large number of macrophages in the process of engulfing the PM_{2.5} particles. We used a dose of 5 mg/kg in our mice base on previous studies (Happo, M. S. et al. 2007; Zhao, C. et al. 2012). The increase in inflammatory cell count in response to PM_{2.5} challenge was found to be dose dependent as the dose of 1 mg/kg did not produce significant increase in cell count in comparison to control mice but the dose of 3 mg/kg produced a statistically significant increase in cell count and a higher response to 10 mg/kg dose (Happo, M. S. et al. 2007). Although PM_{2.5} exacerbation of COPD has been documented in many clinical studies (Faustini, A. et al. 2012; Janssen, N. A. et al. 2002; Zanobettu, A. et al. 2008; Sunyer, J. et al. 2000), there are no reports demonstrating these effects in a COPD-animal model. However in a recent study (Zhao, C. et al. 2012), researchers found that challenging healthy BALB/c mice with intratracheal PM_{2.5} led to down-regulation of TLR4 in BALF for 14 days and up-regulation of TLR4 in peripheral blood mononuclear cells, In addition they reported an imbalance in the Th1/Th2 response that led to Th2-mediated allergic inflammation, manifested both as peribronchiolar and perivascular inflammatory cell infiltration (Zhao, C. et al. 2012). A dose of 40 mg/kg CMC2.24 was used to treat PM2.5 challenged COPD mice. This dose was based on the response obtained in other study in which the dose of 30 mg/kg of CMC 2.24 was used to treat periodontal disease (Elburki, M. S. et al. 2014) and a pneumonia study in which we gave 40 mg/kg. The inhibition, by CMC 2.24 treatment, of the inflammatory changes in COPD-mice challenged with PM_{2.5} demonstrates the anti-inflammatory properties of the compound which is well-known for the parent compound, curcumin. The latter, has been reported to inhibit NF-κB activation by a decrease in the levels of the phosphorylated NF-κB p65 and to inhibit IL-8 release, cyclooxygenase-2 expression, and neutrophil recruitment in the lungs (Avasarala S. et al. 2013; Rahman I. 2008). It also causes inhibition of reactive oxygen species (ROS) and reactive nitrogen species (RNS) (Biswas, S. K. et al. 2005), and shows an increased expression of histone-deacetylase (HDAC) (Balasubramanyam K. et al. 2004; Kang, J. et al. 2005). In our study, CMC 2.24 importantly showed a systemic ability to significantly prevent apoptosis in COPD-mice challenged with PM_{2.5} a result that is supported by our previous work on bacterial pneumonia. In the latter study a significant reduction in apoptotic cell number was noted in the treated mice.

The underlying mechanisms for PM-induced lung injury are still not fully elucidated, but oxidative stress and inflammatory reaction are considered as key events (Dergham, M. et al. 2012). The levels of TNF-α and IL-6 in BAL fluid were determined by ELISA. We chose to measure these cytokines in particular due to their known participation as acute response factors in PM-mediated pro-inflammatory responses (Hiraiwa, K. et al. 2013; Manzano-Leon, N. et al. 2015). The present results show a significant increase in the levels of TNF-α and IL-6 in PM_{2.5} challenged mice which are in concordance with other studies (Dergham, M. et al. 2012; Manzano-Leon, N. et al. 2015; Salcido-Neyoy, M. E. et al. 2015). In vitro exposure of human monocytic cell line to PM_{2.5} and PM₁a showed increase in the levels of TNF-α and IL-6 which varies according to particles size and season of PM collection (Manzano-Leon, N. et al. 2015), and another in vitro study in which BEAS-2B human bronchial epithelial cells exposure to PM resulted in statistically significant increase in gene expression and protein secretion of IL-6 (Dergham, M. et al. 2012).

Matrix metalloproteinases (MMPs) are complex zinc-containing proteolytic enzymes that are generally capable of degrading all components of the extracellular matrix (ECM) and basement membrane both in normal physiological states and abnormal pathological processes. MMPs are released from inflammatory cells (neutrophils and macrophages) in the lung of COPD mice. MMP-2 is secreted as a 72-kDa pro-form that is cleaved into a 64-kDa active form; the corresponding pro- and active-forms of MMP-9 have masses of 92 kDa and 83 kDa, respectively (Ling, S. H. et al. 2009). Our study showed that Elastase/LPS-treated mice showed a significant increase in the activities of MMPs 2, 9, and 12 and that this increase is associated with the emphysematous and inflammatory changes of COPD. A further increase in the activities of these proteinases occurred on exposure of the COPD-mice to PM_{2.5}. By contrast, CMC 2.24-treated mice showed a significant reduction (to essentially normal levels) in the activities of MMPs 2, 9, and 12 which are associated with attenuation of lung injury. This confirms the results of many studies that have outlined the importance of these MMPs in lung injury. A study by Ganesan at al. demonstrated that the administration of quercetin prevents further degradation of alveolar walls by decreasing MMP expression, thereby slowing the progression of emphysema in Elastase/LPS-treated mice (Halber, R. J. et al. 2006). Neutrophil elastase knockout-mice are 60% protected against widening of airspace (emphysema), whereas MMP-12 (macrophage metallo-elastase) knockout-mice are 100% protected (Shapiro, S. D. et al. 2003; Hautamaki, R. D. et al. 1997). Very few studies focused on the role of MMP-12 in development of human COPD. A study by Demedts et al. (Demedts et al. 2006) found that the level of MMP-12 in induced sputum is significantly higher in mild to moderate COPD patients than the control groups which suggest the important role of MMP-12 in the development of COPD in humans and confirm the results from animal studies.

In summary, the results of this study how that CMC 2.24 has the capacity to reduce significantly the Elastase/LPS-induced lung-inflammation and can inhibit tissue (lung parenchyma) destruction. This substance also significantly prevented the exacerbation of the inflammation induced by exposure to PM_{2.5}. The anti-inflammatory and other secondary effects of CMC 2.24 indicate that it has therapeutic potential for the treatment of COPD and COPD exacerbation, especially as it is of extremely low toxicity and is systemically active by oral administration, in contrast to curcumin itself.

### Emphysema

Chronic obstructive pulmonary disease (COPD) is the most common chronic lung disease in adults and is a leading cause of death worldwide (Halbert, R. J. et al. 2006; Tibboel, J. et al. 2014). COPD is a progressive disorder of the lung parenchyma, characterized by chronic inflammation, the plugging of small airways by increased mucus secretion, emphysema, and abnormal inflammatory response to external stimuli (Sajjan, U. et al. 2009; Ganesan S. et al. 2012; Ganesan, S. et al. 2010; Le Quement, C. et al. 2008). Pulmonary emphysema is a condition characterized by alveolar destruction, resulting in a reduced alveolar surface area and increased alveolar size (Tibboel, J. et al. 2014). Although there are many new theories that claim to explain alveolar wall destruction in COPD, the protease-antiprotease hypothesis remains the main thesis. This belief was formulated by the observation that humans deficient in al-antitrypsin (A1AT) developed early emphysema and from animal experiments which showed that instillation of elastolytic enzymes produced emphysema in experimental animal (Churg, A. et al. 2008). Surfactant Protein D (SP-D) is a member of the collectins superfamily, and has an important role in innate host defense as well as immunomodulatory functions (Botas, C. et al. 1998; Korhagen, T. R. et al. 1998). Mice lacking SP-D protein develop an early onset emphysematous phenotype, hypertrophy and hyperplasia of alveolar type II cells, disturbances of surfactant homoeostasis. Accumulation of foamy appearing alveolar macrophages and peribronchial and perivascular infiltrates are typical findings in these mice (Knudsen, L. et al. 2014). The SP-D knockout (KO) mice provides an appropriate model for progressive emphysema at an early age as SP-D KO mice develop emphysema phenotype at the age of 8 weeks and becomes notable by the age of 18 weeks (Botas, C. et al. 1998).

CMC 2.24 prevents the inflammatory processes that lead to progressive alveolar destruction in this mouse emphysema model and reverses the damage already present in older SP-D KO mice.

### Pneumonia

Humanized transgenic (hTG) mouse models is one powerful tool for studying the pathophysiological function of human genetic gene/variants (alleles) in clinically important disease (Shultz, L. D. et al. 2007; Gonzalez, F. J. et al. 2006; Shultz, L. D. et al. 2011). The hTG model can elucidate subtle differences in phenotypes caused by human genetic variants and overcome study design limitations in infection diseases in vivo (Zhang, L. et al. 2007; Lassnig, C. et al. 2005). hTG SP-A mice were recently generated and it was shown that the formation of the tubular myelin (TM) in vivo requires both SP-A1 and SP-A2 gene products (Wang, G. et al. 2010). Thus, hTG mice are an ideal in vivo system to study functional differences in SP-B C and T alleles in bacterial pneumonia. Additionally, to monitor the changes of bacterial dynamic growth we have used bioluminescent labeled S. aureus and an in vivo image system (Pribaz, J. R. et al. 2012; Guo, Y. et al. 2013). The advanced hTG mouse model provides us with a unique opportunity to investigate functional differences of SP-B genetic variants in vivo and to monitor dynamic changes in bacteria growth in our pneumonia model.

Increased evidence indicated that sexual dimorphism affects the rate of disease incidence, onset and associated symptoms (Morrow, E. H. et al. 2015). Sexual dimorphism also leads to altered susceptibility to infectious disease, and differing modulation of innate immune activity, as well as age and sex-specific changes of the immune system (Giefing-Kroll, C. et al. 2015). Consequently, for the ALI/ARDS caused by bacterial pneumonia, there may be variance of susceptibility and bacterial clearance potency.

During infection, increased neutrophil infiltration and lung tissue apoptosis, cytokine synthesis, and degradation of lung matrix result in lung injury severity. Curcumin, is extracted from the rhizomes of the plant cucuma longa, which possesses several pharmacological properties including anti-inflammatory and anti-oxidant effects. Curcumin also selectively inhibits the activities of inducible matrix metalloproteinases (MMPs), and downregulate expression of pro-inflammatory cytokines through modulation of NF-κB and related signaling pathways (Jobin, C. et al. 1999; Xiao, X. et al. 2013). CMC2.24 was developed to enhance bioactivity and bioavailability with decreased toxicity (21). CMC2.24 is also more potent than natural curcumins at inhibition of apoptosis, inflammation, and inducible MMPs, all of which contribute to propagation of lung injury (Zhang, Y. et al. 2012; Corbel, M. et al. 2000). In the present study we have observed differential susceptibility to bacterial pneumonia between hTG SP-B-C and SP-B-T mice and protective effects of CMC2.24 in the lung injury of infected mice.

Pneumonia is the leading cause of infectious morbidity and mortality in the United States (Garibaldi, et al. 1985). It is leading major cause of ALI and ARDS which have very high mortality (40-60%) as well (Rubenfeld, G. D. et al. 2007). Genetic variations of SP-B with subsequent loss of surfactant activity appear to be critical in ARDS progression (Quasney, M. W. et al. 2004; Simonato, M. et al. 2011; Schmidt, R. et al. 2007), which may explain clinically observed differences in morbidity and mortality in patients with pneumonia-induced ARDS. It is unclear why some of individuals are more susceptible to bacterial pneumonia compared with the others. In the present study, we investigated the functional differences of hTG SP-B-C and SP-B-T mice in responses to S. aureus infection with or without CMC 2.24 treatment. We found significantly differential resistance of hTG SP-B-C and SP-B-T mice to bacteria using in vivo imaging method, as well as differential lung injury evidenced by histopathology, cell and molecular analyses. We also demonstrate CMC2.24 attenuates lung injury after bacterial infection by attenuating lung inflammation, apoptosis and MMP activation.

SP-B, a key component of pulmonary surfactant, is essential for normal lung function (36-40). An acute reduction in SP-B by 75-80% causes lethal respiratory failure in animals (Melton, K. R. et al. 2003). Likewise SP-B levels are decreased by up to 60% in patients with acute lung injury and ARDS due to enhanced SP-B turnover and degradation (Simonato, M. et al. 2011). SP-B gene expresses two protein products, SP-B^{M} and SP-B^{M}, involved in lowering surface tension and host defense, respectively (Yang, L. et al. 2010). Although a number of hSP-B polymorphisms and mutations have been identified (Nogee, L. M. et al. 1994; ), the SNP rs1130866 i.e. SP-BC/T1580 is functionally one of the most important. This SP-BC/T1580 polymorphism is not only associated with pneumonia and pneumonia-induced ARDS (Quasney, M. W. et al. 2004; Lin, Z. et al. 2000; Dahmer, M. K. et al. 2011), but also with neonatal respiratory distress syndrome (RDS) (Martilla, R. et al. 2003; Hamvas, A. et al. 2009; Yin, X. et al. 2013) and interstitial lung disease (ILD) (Sumita, Y. et al. 2008). The detailed mechanisms for the increased susceptibility of SP-B C allele to these pulmonary diseases are unknown (Wang, G. at al. 2003; Hamvas, A. et al. 2007; Guttentag, S. et al. 2008). The results of this study indicate SP-B-C mice are more susceptible to bacterial infection with more severe lung injury and inflammation in the lung compared with SP-B-T mice. Because the only difference between SP-B-C and SP-B-T mice is the SP-B gene the differential response to bacterial pneumonia in these two mouse lines is caused by the products of SP-B C and T alleles. We also observed the SP-B level in the BAL fluid of infected SP-B-C mice decreased more than that of infected SP-B-T mice, suggesting difference in SP-B processing and/or degradation in SP-B-C and SP-B-T mice during S. aureus pneumonia.

In vivo imaging system has provided us with a unique tool for monitoring bacterial viability in vivo after bacterial inoculation. Of interest, all the effects of gender on bacterial viability were observed in the present study. Previous studies have shown that the sex hormones can influence the immune response to bacterial infection (Giefing-Kroll, C. et al. 2015). In this study, infected male mice exhibited higher load of bacteria in the early stage of infection compared to infected female mice. However, infected female mice had higher load of bacteria in the lung than infected male mice by 48 h after infection. Sex hormones may contribute these differences.

These results demonstrate CMC2.24 has a protective effect on lung injury in this model of bacterial pneumonia. The protective mechanisms for the effect of CMC2.24 in the current study are its ability to reduce inflammatory cell infiltration at the site of lung infection and prevent apoptosis. The effects of CMC2.24 on pulmonary inflammation and apoptosis are confirmed in bacterial pneumonia by our results. Previous studies also demonstrate that curcumins are involved in the modulation of inflammatory signaling pathways and mediators, including reduction in NF-κB activation and lipid derived inflammatory mediators (55), inhibition of reactive oxygen species (ROS) and reactive nitrogen species (RNS) (Biswas, S. K. et al. 2005), and increased expression of histone deacetylase (HDAC) (Balasubramanyam, K. et al. 2004; Kang, J. et al. 2005). In the present study we observed decreased levels of NF-kB p65 and p-Ikb in the lung tissues of infected mice after CMC2.24 treatment. These results are consistent with the previous observations regarding curcumin's effects in the regulation of inflammation.

MMPs, a group of complex zinc-containing neutral proteolytic enzymes, are essential for the degradation and turnover of component of extracellular matrix (ECM). Owing to pulmonary infection, inducible MMPs can degrade connective tissue and exacerbate various lung injury (Pires-Neto, R. C. et al. 2013). From inflammatory cells in the lung of infected mice, MMP-2 is secreted as a 72-kDa pro-form that is cleaved into a 64-kDa active form; the corresponding pro- and active forms of MMP-9 have masses of 92 kDa and 83 kDa, respectively (Xiao, X. et al. 2012; Corbel, M. et al. 2000; Moghaddam, S. J. et al. 2009). In the present study, the activity of MMP-2, -9, and -12 was induced in BALF of infected mice and attenuated by CMC2.24 treatment. Collectively, these results indicate CMC2.24 may have therapeutic potential in bacterial pneumonia.

In summary, functional differences of human SP-B genetic variants, i.e. the SP-B C and T alleles were observed in the bacterial pneumonia, SP-B-C mice showed more susceptible to S. aurus infection compared to SP-B-T mice. Differentially dynamic loads of bacteria between male and female mice were also observed by in vivo imaging bioluminescence. Finally, CMC2.24 improves mortality and attenuates lung injury in this model of S. aureus pneumonia.

### REFERENCES

Ammon H. P. T.; Wahl M. A. Pharmacology of Curcuma longa. Planta Med, 1991, 57, 1-7.
Avasarala S, Zhang F, Liu G, Wang R, London S D, London L. Curcumin modulates the inflammatory response and inhibits subsequent fibrosis in a mouse model of viral-induced acute respiratory distress syndrome. PloS one 2013; 8(2):e57285.
D. Bai et al. Comparative effectiveness of cefazolin versus cloxacillin as definitive antibiotic therapy for MSSA bacteraemia: results from a large multicentre cohort study. J Antimicrob Chemother 70(5):1539-46, 2015b.
D. Bai et al. Impact of Infectious Disease Consultation on Quality of Care, Mortality, and Length of Stay in Staphylococcus aureus Bacteremia: Results From a Large Multicenter Cohort Study. Clin Infect Dis, 2015a.
Balasubramanyam K, et al. Curcumin, a novel p300/creb-binding protein-specific inhibitor of acetyltransferase, represses the acetylation of histone/nonhistone proteins and histone acetyltransferase-dependent chromatin transcription. The Journal of biological chemistry 2004; 279(49):51163-51171.
M. Balasubramanyam, et al. Curcumin-induced inhibition of cellular reactive oxygen species generation: novel therapeutic implications. J Biosci 28(6):715-21, 2003.
Balode L l, et al. Lipoxygenase-derived arachidonic acid metabolites in chronic obstructive pulmonary disease. Medicina (Kaunas). 2012, 48(6), 292-8.
Bernstein, A. S. and H. T. Abelson, PM 2.5—a killer in our midst. Archives of pediatrics 6 adolescent medicine, 2005. 159(8): p. 786.
S. K. Biswas, et al. Curcumin induces glutathione biosynthesis and inhibits NF-kappaB activation and interleukin-8 release in alveolar epithelial cells: mechanism of free radical scavenging activity. Antioxid Redox Signal 7(1-2):32-41, 2005.
Bonnans C, et al. Lipoxins are potential endogenous antiinflammatory mediators in asthma. Am J Respir Crit Care Med. 2002, 165(11), 1531-5.
Botas, C., et al., Altered surfactant homeostasis and alveolar type II cell morphology in mice lacking surfactant protein D. Proceedings of the National Academy of Sciences of the United States of America, 1998. 95(20): p. 11869-74.
Botchkina, G. I., et al., Prostate cancer stem cell-targeted efficacy of a new-generation taxoid, SBT-1214 and novel polyenolic zinc-binding curcuminoid, CMC2.24. PloS one, 2013. 8(9): p. e69884.
Bratcher, P. E., et al., MMP-9 cleaves SP-D and abrogates its innate immune functions in vitro. PloS one, 2012. 7(7): p. e41881.
Buckley C. D, Gilroy D. W, Serhan C. N. Proresolving Lipid Mediators and Mechanisms in the Resolution of Acute Inflammation. Immunity 2014, 40 (3), 315-27.
Burney, P., et al., Chronic obstructive pulmonary disease mortality and prevalence: the associations with smoking and poverty—a BOLD analysis. Thorax, 2014. 69(5): p. 465-73.
J. C. Clark, et al. Targeted disruption of the surfactant protein B gene disrupts surfactant homeostasis, causing respiratory failure in newborn mice. Proc Natl Acad Sci USA 92(17):7794-8, 1995.
C. G. Clement et al. Stimulation of lung innate immunity protects against lethal pneumococcal pneumonia in mice. Am J Respir Crit Care Med 177(12):1322-30, 2008
M. Corbel, et al. Role of gelatinases MMP-2 and MMP-9 in tissue remodeling following acute lung injury. Braz J Med Biol Res 33(7):749-54, 2000.
Crouch, E. & J. R. Wright, Surfactant proteins A and D and pulmonary host defense. Annual review of physiology, 2001. 63: 521-54
Churg, A., M. Cosio, and J. L. Wright, Mechanisms of cigarette smoke-induced COPD: insights from animal models. American journal of physiology. Lung cellular and molecular physiology, 2008. 294(4): p. L612-31.
M. K. Dahmer et al. The influence of genetic variation in surfactant protein B on severe lung injury in African American children. Crit Care Med 39(5):1138-44, 2011.
Deacon, R. M., Measuring the strength of mice. Journal of visualized experiments: JoVE, 2013(76).
De Brauwer, E. I., et al., Bronchoalveolar lavage fluid differential cell count. How many cells should be counted?Analytical and quantitative cytology and histology/the International Academy of Cytology [and] American Society of Cytology, 2002. 24(6): p. 337-41.
Demedts I K et al. Elevated mmp-12 protein levels in induced sputum from patients with copd. Thorax 2006; 61(3):196-201.
D. E. deMello and Z. Lin: Pulmonary alveolar proteinosis: a review. Pediatr Pathol Mol Med 20(5):413-32, 2001.
D. E. deMello, et al. Molecular and phenotypic variability in the congenital alveolar proteinosis syndrome associated with inherited surfactant protein B deficiency. J Pediatr 125(1):43-50, 1994.
Dergham M, et al. Prooxidant and proinflammatory potency of air pollution particulate matter (pm(2).(5)(-)(0).(3)) produced in rural, urban, or industrial surroundings in human bronchial epithelial cells (beas-2b). Chem Res Toxicol 2012; 25(4):904-919.
Elburki M S, et al. A novel chemically modified curcumin reduces severity of experimental periodontal disease in rats: Initial observations. Mediators of inflammation 2014; 2014:959471.
Elkington, P. T. & J. S. Friedland, Matrix metalloproteinases in destructive pulmonary pathology. Thorax, 2006, 61(3), 259-66.
Faustini, A., et al., Short-term effects of air pollution in a cohort of patients with chronic obstructive pulmonary disease. Epidemiology, 2012. 23(6): p. 861-79.
C. W. Farnsworth, C. T. Shehatou, R. Maynard, K. Nishitani, S. L. Kates, M. J. Zuscik, E. M. Schwarz, J. L. Daiss and R. A. Mooney: A Humoral Immune Defect Distinguishes the Response to S. aureus Infections in obesity and Type 2 Diabetes from Type 1 Diabetes. Infect Immun, 2015.
J. Floros and P. Kala: Surfactant proteins: molecular genetics of neonatal pulmonary diseases. Annu Rev Physiol 60:365-84, 1998.
Frederick, A. L., T. P. Saborido, and G. D. Stanwood, Neurobehavioral phenotyping of G(alphaq) knockout mice reveals impairments in motor functions and spatial working memory without changes in anxiety or behavioral despair. Frontiers in behavioral neuroscience, 2012. 6: p. 29.
Ganesan, S., et al., Elastase/LPS-exposed mice exhibit impaired innate immune responses to bacterial challenge: role of scavenger receptor A. The American journal of pathology, 2012. 180(1): p. 61-72.
Ganesan, S., et al., Quercetin prevents progression of disease in elastase/LPS-exposed mice by negatively regulating MMP expression. Respiratory research, 2010. 11: p. 131.
R. A. Garibaldi: Epidemiology of community-acquired respiratory tract infections in adults. Incidence, etiology, and impact. Am J Med 78(6B):32-7, 1985.
C. Giefing-Kroll, P. Berger, G. Lepperdinger and B. Grubeck-Loebenstein: How sex and age affect immune responses, susceptibility to infections, and response to vaccination. Aging Cell 14(3):309-21, 2015.
Gonzalez and A. M. Yu: Cytochrome P450 and xenobiotic receptor humanized mice. Annu Rev Pharmacol Toxicol 46:41-64, 2006.
W. A. Gower and L. M. Nogee: Surfactant dysfunction. Paediatr Respir Rev 12(4):223-9, 2011.
Guenther, K., et al., Early behavioural changes in scrapie-affected mice and the influence of dapsone. The European journal of neuroscience, 2001. 14(2): p. 401-9.
Y. Guo, et al. In vivo bioluminescence imaging to evaluate systemic and topical antibiotics against community-acquired methicillin-resistant Staphylococcus aureus-infected skin wounds in mice. Antimicrob Agents Chemother 57(2):855-63, 2013.
Gupta S. C. et al. Multitargeting by curcumin as revealed by molecular interaction studies. Natural Products Reports, 2011, 28, 1937-1955.
S. Guttentag: Posttranslational regulation of surfactant protein B expression. Semin Perinatol 32(5):367-70, 2008.
Haibert, R. J., et al., Global burden of COPD: systematic review and meta-analysis. The European respiratory journal, 2006. 28(3): p. 523-32.
A. Hamvas, F. S. Cole and L. M. Nogee: Genetic disorders of surfactant proteins. Neonatology 91(4):311-7, 2007.
Hamvas, H. B. et al. Developmental and genetic regulation of human surfactant protein B in vivo. Neonatology 95(2):117-24, 2009.
Happo M S, et al. Dose and time dependency of inflammatory responses in the mouse lung to urban air coarse, fine, and ultrafine particles from six european cities. Inhal Toxicol 2007; 19(3):227-246.
Hautamaki R D, Kobayashi D K, Senior R M, Shapiro S D. Requirement for macrophage elastase for cigarette smoke-induced emphysema in mice. Science 1997; 277(5334):2002-2004.
Hiraiwa K, van Eeden S F. Contribution of lung macrophages to the inflammatory responses induced by exposure to air pollutants. Mediators Inflamm 2013; 2013:619523.
Janssen N A, Schwartz J, Zanobetti A, Suh H H. Air conditioning and source-specific particles as modifiers of the effect of pm(10) on hospital admissions for heart and lung disease. Environmental health perspectives 2002; 110(1):43-49.
C. Jobin, et al. Curcumin blocks cytokine-mediated NF-kappa B activation and proinflammatory gene expression by inhibiting inhibitory factor I-kappa B kinase activity. J Immunol 163(6):3474-83, 1999.
Kang J, Chen j, Shi Y, Jia J, Zhang Y. Curcumin-induced histone hypoacetylation: The role of reactive oxygen species. Biochemical pharmacology 2005; 69(8):1205-1213.
Kappos, A. D., et al., Health effects of particles in ambient air. International journal of hygiene and environmental health, 2004. 207(4): p. 399-407.
Karp C L, et al. Defective lipoxin-mediated anti-inflammatory activity in the cystic fibrosis airway. Nat Immunol. 2004, 5(4), 388-92.
R. M. Klevens, et al. Active Bacterial Core surveillance: Invasive methicillin-resistant Staphylococcus aureus infections in the United States. JAMA 298(15):1763-71, 2007.
Knudsen, L., et al., Assessment of air space size characteristics by intercept (chord) measurement: an accurate and efficient stereological approach. Journal of applied physiology, 2010. 108(2): p. 412-21.
Knudsen, L., et al., NOS2 is critical to the development of emphysema in Sftpd deficient mice but does not affect surfactant homeostasis. PloS one, 2014. 9(1): p. e85722.
Korfhagen, T. R., et al., Surfactant protein-D regulates surfactant phospholipid homeostasis in vivo. The Journal of biological chemistry, 1998. 273(43): p. 28438-43.
Kurhanewicz, N., et al., Ozone co-exposure modifies cardiac responses to fine and ultrafine ambient particulate matter in mice: concordance of electrocardiogram and mechanical responses. Particle and fibre toxicology, 2014. 11(1): p. 54.
C. Lassnig, A. Kolb, B. Strobl, L. Enjuanes and M. Muller: Studying human pathogens in animal models: fine tuning the humanized mouse. Transgenic Res 14(6):803-6, 2005.
Le Quement, C., et al., The selective MMP-12 inhibitor, AS111793 reduces airway inflammation in mice exposed to cigarette smoke. British journal of pharmacology, 2008. 154(6): p. 1206-15.
Z. Lin, et al. An SP-B gene mutation responsible for SP-B deficiency in fatal congenital alveolar proteinosis: evidence for a mutation hotspot in exon 4. Mol Genet Metab 64(1):25-35, 1998.
Z. Lin, et al. Polymorphisms of human SP-A, SP-B, and SP-D genes: association of SP-B Thr131Ile with ARDS. Clin Genet 58(3):181-91, 2000.
Ling, S. H. and S. F. van Eeden, Particulate matter air pollution exposure: role in the development and exacerbation of chronic obstructive pulmonary disease. International journal of chronic obstructive pulmonary disease, 2009. 4: p. 233-43.
J. Liu, et al. Role of surfactant proteins A and D in sepsis-induced acute kidney injury. Shock 43(1):31-8, 2015.
C. C. Ma and S. Ma: The role of surfactant in respiratory distress syndrome. Open Respir Med J 6:44-53, 2012.
Malloy, J., et al., Alterations of the endogenous surfactant system in septic adult rats. American journal of respiratory and critical care medicine, 1997. 156(2 Pt 1): p. 617-23.
Manzano-Leon N, et al. Tnf-alpha and i1-6 responses to particulate matter: Variation according to pm size, season, and polycyclic aromatic hydrocarbon and soil content. Environ Health Perspect 2015.
R. Marttila, et al. Surfactant protein A and B genetic variants in respiratory distress syndrome in singletons and twins. Am J Respir Crit Care Med 168(10):1216-22, 2003.
Marumo, S., et al., p38 mitogen-activated protein kinase determines the susceptibility to cigarette smoke-induced emphysema in mice. BMC pulmonary medicine, 2014. 14: p. 79.
Matute-Bello, G., et al., An official American Thoracic Society workshop report: features and measurements of experimental acute lung injury in animals. American journal of respiratory cell and molecular biology, 2011. 44(5): p. 725-38.
K. K. Meja, et al. Curcumin restores corticosteroid function in monocytes exposed to oxidants by maintaining HDAC2. Am J Respir Cell Mol Biol 39(3):312-23, 2008.
Mock M.; Fouet A. Anthrax. Annual Review of Microbiology, 2001, 55, 647-671.
K. R. Melton, et al. SP-B deficiency causes respiratory failure in adult mice. Am J Physiol Lung Cell Mol Physiol 285(3):L543-9, 2003.
Min, T., et al., Critical role of proteostasis-imbalance in pathogenesis of COPD and severe emphysema. Journal of molecular medicine, 2011. 89(6): p. 577-93.
Mock M.; Mignot T. Anthrax toxins and the host: a story of intimacy. Cellular Microbiology, 2003, 5(1), 15-23.
S. J. Moghaddam, et al. Curcumin inhibits COPD-like airway inflammation and lung cancer progression in mice. Carcinogenesis 30(11):1949-56, 2009.
E. H. Morrow: The evolution of sex differences in disease. Biol Sex Differ 6:5, 2015.
Murray, C. J. and A. D. Lopez, Alternative projections of mortality and disability by cause 1990-2020: Global Burden of Disease Study. Lancet, 1997. 349(9064): p. 1498-504.
L. M. Nogee, et al. A mutation in the surfactant protein B gene responsible for fatal neonatal respiratory disease in multiple kindreds. J Clin Invest 93(4):1860-3, 1994.
Ostro, B., et al., The effects of components of fine particulate air pollution on mortality in California: results from CALFINE. Environmental health perspectives, 2007. 115(1): p. 13-9.
M. Otto: Basis of virulence in community-associated methicillin-resistant Staphylococcus aureus. Annu Rev Microbiol 64:143-62, 2010.
Parkinson J F. Lipoxin and synthetic lipoxin analogs: an overview of anti-inflammatory functions and new concepts in immunomodulation. Inflamm Allergy Drug Targets. 2006, 5(2), 91-106.
R. C. Pires-Neto, et al. Expression of acute-phase cytokines, surfactant proteins, and epithelial apoptosis in small airways of human acute respiratory distress syndrome. J Crit Care 28(1):111 e9-111 e15, 2013.
Planagumà A, et al. Airway lipoxin A4 generation and lipoxin A4 receptor expression are decreased in severe asthma. Am J Respir Crit Care Med. 2008, 178(6), 574-82.
Poulain, F. R., et al., Ultrastructure of phospholipid mixtures reconstituted with surfactant proteins B and D. American journal of respiratory cell and molecular biology, 1999. 20(5): p. 1049-58.
J. R. Pribaz, et al. Mouse model of chronic post-arthroplasty infection: noninvasive in vivo bioluminescence imaging to monitor bacterial burden for long-term study. J Orthop Res 30(3):335-40, 2012.
M. W. Quasney, et al. Association between surfactant protein B+1580 polymorphism and the risk of respiratory failure in adults with community-acquired pneumonia. Crit Care Med 32(5):1115-9, 2004.
Rahman, I., Antioxidant therapeutic advances in COPD. Therapeutic advances in respiratory disease, 2008. 2(6): p. 351-74.
Rahman, I., Antioxidant therapies in COPD. International journal of chronic obstructive pulmonary disease, 2006. 1(1): p. 15-29.
Riva, D. R., et al., Low dose of fine particulate matter (PM2.5) can induce acute oxidative stress, inflammation and pulmonary impairment in healthy mice. Inhalation toxicology, 2011. 23(5): p. 257-67.
J. Rowe, et al. Compounds that target host cell proteins prevent varicella-zoster virus replication in culture, ex vivo, and in SCID-Hu mice. Antiviral Res 86(3):276-85, 2010.
G. D. Rubenfeld and M. S. Herridge: Epidemiology and outcomes of acute lung injury. Chest 131(2):554-62, 2007.
Sajjan, U., et al., Elastase- and LPS-exposed mice display altered responses to rhinovirus infection. American journal of physiology. Lung cellular and molecular physiology, 2009. 297(5): p. L931-44.
Salcido-Neyoy M E, et al. induction of c-jun by air particulate matter (pm(1)(0)) of Mexico city: Participation of polycyclic aromatic hydrocarbons. Environ Pollut 2015; 203:175-182.
R. Schmidt, et al. Time-dependent changes in pulmonary surfactant function and composition in acute respiratory distress syndrome due to pneumonia or aspiration. Respir Res 8:55, 2007.
M. P. Schreiber, et al. Bacteremia in Staphylococcus aureus pneumonia: outcomes and epidemiology. J Crit Care 26(4):395-401, 2011.
Serhan C N, et al. Lipoxins: novel series of biologically active compounds formed from arachidonic acid in human leukocytes. Proc Natl Acad Sci USA, 1984, 81(17), 5335-5339.
Shapiro S D, et al. Neutrophil elastase contributes to cigarette smoke-induced emphysema in mice. The American journal of pathology 2003; 163(6):2329-2335.
Shishodia, S., et al., Curcumin (diferuloylmethane) down-regulates cigarette smoke-induced NF-kappaB activation through inhibition of IkappaBalpha kinase in human lung epithelial cells: correlation with suppression of COX-2, MMP-9 and cyclin D1. Carcinogenesis, 2003. 24(7): p. 1269-79.
Suzuki, M., et al., Curcumin attenuates elastase- and cigarette smoke-induced pulmonary emphysema in mice. American journal of physiology. Lung cellular and molecular physiology, 2009. 296(4): p. L614-23.
Sohn, S. H., et al., The effects of Gamijinhae-tang on elastase/lipopolysaccharide-induced lung inflammation in an animal model of acute lung injury. BMC complementary and alternative medicine, 2013. 13(1): p. 176.
L. D. Shultz, et al. Humanized mice in translational biomedical research. Nat Rev Immunol 7(2):118-30, 2007.
L. D. Shultz, et al. Humanized mice as a preclinicai tool for infectious disease and biomedical research. Ann N Y Acad Sci 1245:50-4, 2011.
M. Simonato, et al. Disaturated-phosphatidylcholine and surfactant protein-B turnover in human acute lung injury and in control patients. Respir Res 12:36, 2011.
Sreejayan and M. N. Rao: Nitric oxide scavenging by curcuminoids. J Pharm Pharmacol 49(1):105-7, 1997.
Y. Sumita, et al. Genetic polymorphisms in the surfactant proteins in systemic sclerosis in Japanese: T/T genotype at 1580 C/T (Thr131Ile) in the SP-B gene reduces the risk of interstitial lung disease. Rheumatology (Oxford) 47(3):289-91, 2008.
Sunyer J, et al. Patients with chronic obstructive pulmonary disease are at increased risk of death associated with urban particle air pollution: A case-crossover analysis. American journal of epidemiology 2000; 151(1):50-56.
Suzuki, M., et al., Curcumin attenuates elastase- and cigarette smoke-induced pulmonary emphysema in mice. American journal of physiology. Lung cellular and molecular physiology, 2009. 296(4): p. L614-23.
Tibboel, J., et al., Intravenous and intratracheal mesenchymal stromal cell injection in a mouse model of pulmonary emphysema. COPD, 2014. 11(3): p. 310-8.
K. Tokieda, et al. Pulmonary dysfunction in neonatal SP-B-deficient mice. Am J Physiol 273(4 Pt 1):L875-82, 1997.
Vestbo, J., et al., Global strategy for the diagnosis, management, and prevention of chronic obstructive pulmonary disease: GOLD executive summary. American journal of respiratory and critical care medicine, 2013. 187(4): p. 347-65.
Visse, R. and H. Nagase, Matrix metalloproteinases and tissue inhibitors of metalloproteinases: structure, function, and biochemistry. Circulation research, 2003. 92(8): p. 827-39.
G. Wang, et al. Guttentag and J. Floros: Differences in N-linked glycosylation between human surfactant protein-B variants of the C or T allele at the single-nucleotide polymorphism at position 1590: implications for disease. Biochem J 369(Pt 1):179-84, 2003.
G. Wang, et al. Humanized SFTPA1 and SFTPA2 transgenic mice reveal functional divergence of SP-A1 and SP-A2: formation of tubular myelin in vivo requires both gene products. J Biol Chem 285(16):11998-2010, 2010.
X. Wang, et al. The curcumin analogue hydrazinocurcumin exhibits potent suppressive activity on carcinogenicity of breast cancer cells via STAT3 inhibition. Int J Oncol 40(4):1189-95, 2012.
J. A. Whitsett, et al. Alveolar surfactant homeostasis and the pathogenesis of pulmonary disease. Annu Rev Med 61:105-19, 2010.
Wittebole, X. et al. Toll-like receptor 4 modulation as a strategy to treat sepsis. Mediators of inflammation, 2010. 2010: p. 568396.
Wright, J. L. and A. Churg, Cigarette smoke causes physiologic and morphologic changes of emphysema in the guinea pig. The American review of respiratory disease, 1990. 142(6 Pt 1): p. 1422-8.
Wright, J. R., Immunoregulatory functions of surfactant proteins. Nature reviews. Immunology, 2005. 5(1): p. 58-68.
Wu, H., et al., Surfactant proteins A and D inhibit the growth of Gram-negative bacteria by increasing membrane permeability. The Journal of clinical investigation, 2003. 111(10): p. 1589-602.
X. Xiao, et al. Curcumin protects against sepsis-induced acute lung injury in rats. J Surg Res 176(1):e31-9, 2012.
Yamazoe, M., et al., Pulmonary surfactant protein D inhibits lipopolysaccharide (LPS)-induced inflammatory cell responses by altering LPS binding to its receptors. The Journal of biological. chemistry, 2008. 283(51): p. 35878-88.
L. Yang, et al. Surfactant protein B propeptide contains a saposin-like protein domain with antimicrobial activity at low pH. Journal of immunology 194(2):975-83, 2010.
X. Yin, et al. Surfactant protein B deficiency and gene mutations for neonatal respiratory distress syndrome in China Han ethnic population. Int J Clin Exp Pathol 6(2):267-72, 2013.
Zanobetti, A. et al. Particulate air pollution and survival in a COPD cohort. Environmental health: a global access science source, 2008, 7, 48.
L. Zhang, G. I. Kovalev and L. Su: HIV-1 infection and pathogenesis in a novel humanized mouse model. Blood 109(7):2978-81, 2007.
Zhang Y. et al. pKa, Zinc- and Serum Albumin-Binding of Curcumin and Two Novel Biologically-Active, Chemically-Modified Curcumins. Current Medicinal Chemistry, 2012, 19(25), 4367-4375.
Zhang Y. et al. Design, Synthesis, and Biological Activity of New Polyenolic Inhibitors of Matrix Metalloproteinases: A Focus on Chemically-Modified Curcumins. Current Medicinal Chemistry, 2012, 19(25), 4348-4358.
Zhao C, et al. Involvement of t1r2 and t1r4 and th1/th2 shift in inflammatory responses induced by fine ambient particulate matter in mice. Inhalation toxicology 2012; 24(13):918-927.

1-58. (canceled)

59. A method of treating a subject afflicted with a disease or condition and increasing production of at least one of lipoxin, resolvin, protectin, and maresin in a subject in need thereof, said method comprising: administering to the subject an amount of a compound having the structure: 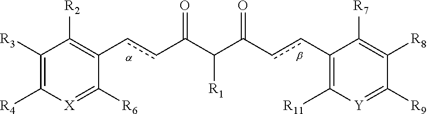
or a pharmaceutically acceptable salt or ester thereof,
wherein bond α and β are each, independently, present or absent;
X is CR₅, or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃ —SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₅R₁₇, or 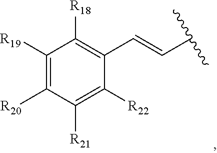
wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocycyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and wherein each occurrence of alkyl, alkenyl, or alkynyl is branched or unbranched, unsubstituted or substituted.

60. The method of claim 59, wherein the method increases the production of the at least one lipoxin in the subject.

61. The method of claim 59, wherein the at least one lipoxin is selected from lipoxin A4, 15-epi-LXA4 and lipoxin B4.

62. The method of claim 59, wherein the method increases the production of the at least one resolvin in the subject.

63. The method of claim 59, wherein the at least one resolvin is selected from RvE1, RvE2, RvE3, RvD1, RvD2, RvD3, RvD4 and RvD5.

64. The method of claim 59, wherein the method increases the production of the at least one protectin in the subject.

65. The method of claim 59, wherein the at least one protectin is PD1-NPD1.

66. The method of claim 59, wherein the method increases the production of the at least one maresin in the subject.

67. The method of claim 59, wherein the at least one maresin is MaR1.

68. The method of claim 59, wherein the compound is at least one selected from the group consisting of:

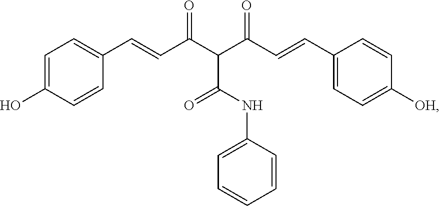 [SMILES: O=C(/C=C/c1ccc(O)cc1)C(C(=O)/C=C/c1ccc(O)cc1)C(=O)Nc1ccccc1]

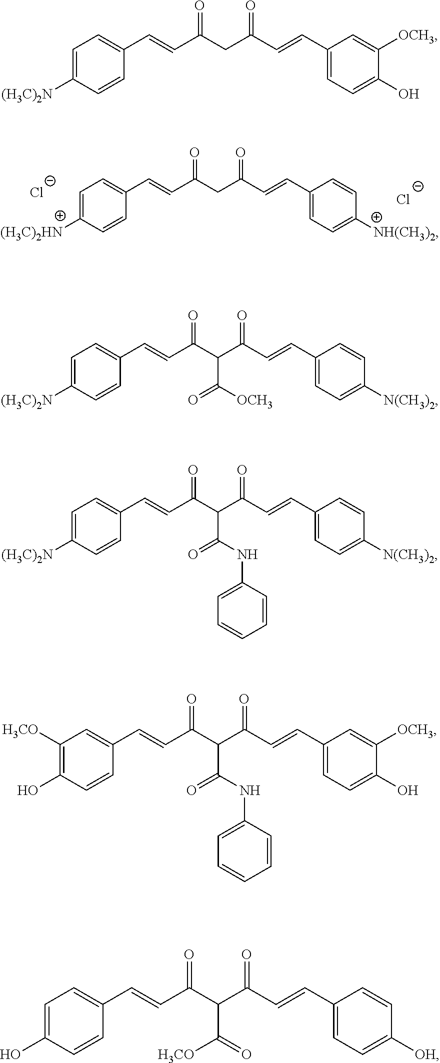

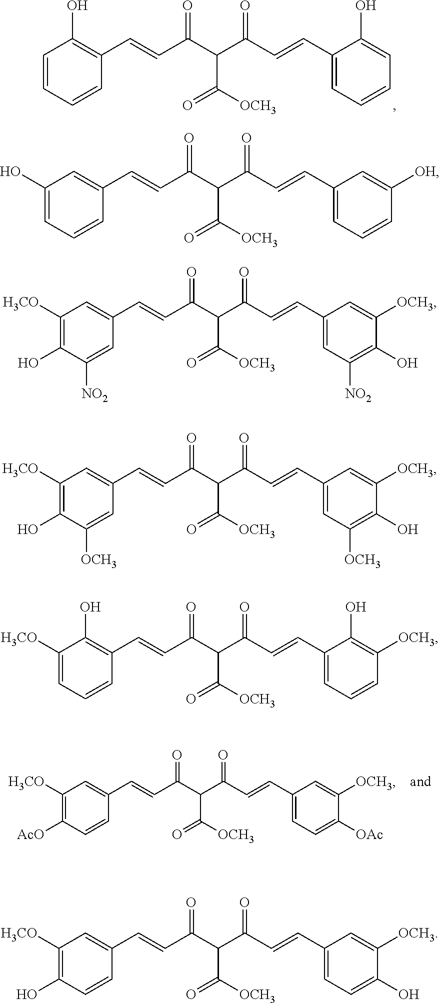

69. The method of claim 59, wherein the compound is 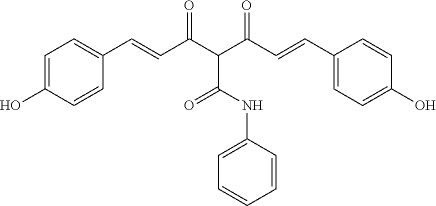 [SMILES: O=C(/C=C/c1ccc(O)cc1)C(C(=O)/C=C/c1ccc(O)cc1)C(=O)Nc1ccccc1]
or a pharmaceutically acceptable salt thereof.

70. The method of claim 59, wherein the disease or condition is acute respiratory distress syndrome (ARDS).

71. The method of claim 68, wherein the disease or condition is acute respiratory distress syndrome (ARDS).

72. The method of claim 69, wherein the disease or condition is acute respiratory distress syndrome (ARDS).
